SEQUENCE LISTING (27 sequences)

SEQ ID NO: 1 - DNA, 1443 bp, Homo sapiens
  CDS (3)..(1046)
acccacgcgtccgctccatcctagtggcgctgtgcctgtggctgcacctggcgctgggcgtgcgcggcgcgccctgcgaggcggtgcgcatccctatgtgccggcacatgccctggaacatcacgcggatgcccaaccacctgcaccacagcacgcaggagaacgccatcctggccatcgagcagtacgaggagctggtggacgtgaactgcagcgccgtgctgcgcttcttcctctgtgccatgtacgcgcccatttgcaccctggagttcctgcacgaccctatcaagccgtgcaagtcggtgtgccaacgcgcgcgcgacgactgcgagcccctcatgaagatgtacaaccacagctggcccgaaagcctggcctgcgacgagctgcctgtctatgaccgtggcgtgtgcatctcgcctgaagccatcgtcacggacctcccggaggatgttaagtggatagacatcacaccagacatgatggtacaggaaaggcctcttgatgttgactgtaaacgcctaagccccgatcggtgcaagtgtaaaaaggtgaagccaactttggcaacatatctcagcaaaaactacagctatgttattcatgccaaaataaaagctgtgcagaggagtggctgcaatgaggtcacaacggtggtggatgtaaaagagatcttcaagtcctcatcacccatccctcgaactcaagtcccgctcattacaaattcttcttgccagtgtccacacatcctgccccatcaagatgttctcatcatgtgttacgagtggcgctcaaggatgatgcttcttgaaaattgcttagttgaaaaatggagagatcagcttagtaaaagatccatacagtgggaagagaggctgcaggaacagcggagaacagttcaggacaagaagaaaacagccgggcgcaccagtcgtagtaatccccccaaaccaaagggaaagactcctgctcccaaaccagccagtcccaagaagaacattaaaactaggagtgcccagaagagaacaaacccgaaaagagtgtgagctaactagtttccaaagcggagacttccgacttccttacaggatgaggctgggcattgcctgggacagcctatgtaaggccatgtgccccttgccctaacaactcactgcagtgctcttcatagacacatcttgcagcatttttcttaaggctatgcttcagtttttctttgtaagccatcacaagccatagtggtaggtttgccctttggtacagaaggtgagttaaagctggtggaaaaggcttattgcattgcattcaaagtaacctgtgtgcatactctasaaragtagggaaaataatgcttgttacaattctacctaatatgtgcattgtataataaatgccatatttcagacaaaanacgtaattctnttacagtatgtctcactaccc

SEQ ID NO: 2 - protein, 347 aa, Homo sapiens
PRVRSILVALCLWLHLALGVRGAPCEAVRIPMCRHMPWNITRMPNHLHHSTQENAILAIEQYEELVDVNCSAVLRFFLCAMYAPICTLEFLHDPIKPCKSVCQRARDDCEPLMKMYNHSWPESLACDELPVYDRGVCISPEAIVTDLPEDVKWIDITPDMMVQERPLDVDCKRLSPDRCKCKKVKPTLATYLSKNYSYVIHAKIKAVQRSGCNEVTTVVDVKEIFKSSSPIPRTQVPLITNSSCQCPHILPHQDVLIMCYEWRSRMMLLENCLVEKWRDQLSKRSIQWEERLQEQRRTVQDKKKTAGRTSRSNPPKPKGKTPAPKPASPKKNIKTRSAQKRTNPKRV

SEQ ID NO: 3 - DNA, 1581 bp, Homo sapiens
  CDS (78)..(1184)
  mat_peptide (207)..(1181)
  sig_peptide (78)..(206)
  misc_feature (1549): n equals a, t, g, or c
gctgccgactggagtttgggggaagaaactctcctgcgccccagaggatttcttcctcggcgaagggacagcgaaagatgagggtggcaggaagagaagggcgctttctgtctgccggggtcgcagcgcgagaaggcagtgccatgttcctctccatcctagtggcgctgtgcctgtggctgcacctggcgctgggcgtgcgcggcgcgccctgcgaggcggtgcgcatccctatgtgccggcacatgccctggaacatcacgcggatgcccaaccacctgcaccacagcacgcaggagaacgccatcctggccatcgagcagtacgaggagctggtggacgtgaactgcagcgccgtgctgcgcttcttcctctgtgccatgtacgcgcccatttgcaccctggagttcctgcacgaccctatcaagccgtgcaagtcggtgtgccaacgcgcgcgcgacgactgcgagcccctcatgaagatgtacaaccacagctggcccgaaagcctggcctgcgacgagctgcctgtctatgaccgtggcgtgtgcatctcgcctgaagccatcgtcacggacctcccggaggatgttaagtggatagacatcacaccagacatgatggtacaggaaaggcctcttgatgttgactgtaaacgcctaagccccgatcggtgcaagtgtaaaaaggtgaagccaactttggcaacatatctcagcaaaaactacagctatgttattcatgccaaaataaaagctgtgcagaggagtggctgcaatgaggtcacaacggtggtggatgtaaaagagatcttcaagtcctcatcacccatccctcgaactcaagtcccgctcattacaaattcttcttgccagtgtccacacatcctgccccatcaagatgttctcatcatgtgttacgagtggcgctcaaggatgatgcttcttgaaaattgcttagttgaaaaatggagagatcagcttagtaaaagatccatacagtgggaagagaggctgcaggaacagcggagaacagttcaggacaagaagaaaacagccgggcgcaccagtcgtagtaatccccccaaaccaaagggaaagactcctgctcccaaaccagccagtcccaagaagaacattaaaactaggagtgcccagaagagaacaaacccgaaaagagtgtgagctaactagtttccaaagcggagacttccgacttccttacaggatgaggctgggcattgcctgggacagcctatgtaaggccatgtgccccttgccctaacaactcactgcagtgctcttcatagacacatcttgcagcatttttcttaaggctatgcttcagtttttctttgtaagccatcacaagccatagtggtaggtttgccctttggtacagaaggtgagttaaagctggtggaaaaggcttattgcattgcattcaaagtaacctgtgtgcatactctasaaragtagggaaaataatgcttgttacaattctacctaatatgtgcattgtataataaatgccatatttcagacaaaanacgtaattctnttacagtatgtctcactaccc

SEQ ID NO: 4 - protein, 368 aa, Homo sapiens [sequence omitted]

SEQ ID NO: 5 - protein, 325 aa, Bos taurus
MVCGSRGGMLLLPAGLLALAALCLLRVPGARAAACEPVRIPLCKSLPWNMTKMPNHLHHSTQANAILAIEQFEGLLGTHCSPDLLFFLCAMYAPICTIDFQHEPIKPCKSVCERARQGCEPILIKYRHSWPESLACEELPVYDRGVCISPEAIVTADGADFPMDSSNGNCRGASSERCKCKPVRATQKTYFRNNYNYVIRAKVKEIKTKCHDVTAVVEVKEILKASLVNIPRETVNLYTSSGCLCPPLNVNEEYLIMGYEDEERSRLLLVEGSIAEKWKDRLGKKVKRWDMKLRHLGLNTSDSSHSDSTQSQKPGRNSNSRQARN

SEQ ID NO: 6 - DNA, 259 bp, Homo sapiens
  misc_feature (187): n equals a, t, g or c
tgaagccaactttggcaacatatctcagcaaaaactacagctatgttattcatgccaaaataaaagctgtgcagaggagtggctgcaatgaggtcacaacggtggtggatgtaaaagagatcttcaagtcctcatcacccatccctcgaactcaagtcccgctcattacaaattcttcttgccagtntccacacatcctgccccatcaagatgttctcatcatgtgttacggagtggcgctcaaggatgatgcttcttg

SEQ ID NO: 7 - DNA, 321 bp, Homo sapiens
  misc_feature (5): n equals a, t, g, or c
ggcanagctggagttcctgcacgaccctatcaagccgtggaagtcggtgtgccaacgcgcgccaacggactgcaagcccctcatgaagatgtacaaccacagctggcccgaaagcctggcctgagaacgagctgcctgtctatgaaccgtggcgtgtgcatctngcctggaagccatcgtcacggacctcccggagaatgttaagtggatagacatcacaccagacatgatggtacaggaaaggcctcttgatgttgactgtnaaacgnctnagccccgatcggtgcaagtntanaaaggttgaagcccnacttttggnaa

SEQ ID NO: 8 - DNA, 487 bp, Homo sapiens
  misc_feature (5): n equals a, t, g, or c
ggcanagagaagaagggcgctttctgtctgccggggtcgcagcgganaagggcagtgccatgttcctctccatcctggccatcgagcagtacgaggagctggtggacgtgaactgcagcgccgtgctgcgcttcttcctctgtgcccatgtacgcgcccatttgcaccctggagttcctgcacgaccctatcaagccgtgcaagtcggtgtgccaagcgcgcngaacgactgcgaagcccctcatgaagatgtacaaccacagctggcccgaaagcctggcctgngaacgagctgcctgtttatgaaccgtgggcgtgtgcatctggcctgaagccattcgttcaaggaccttcccggaggattgtttaagttggntagacatcaacaccagacatgntggtacaggaaaaggcttttttaatgtttaatttnaaaaggcttanacccntttggtgnaaattttaaaaaggtnaggccaattttngg

SEQ ID NO: 9 - DNA, 339 bp, Homo sapiens
  misc_feature (5): n equals a, t, g, or c
ggcanagagagaagggcgctttctgtctgccggggtcgcagcggagnagggnagtgccatgttcctctccatcctggccatcgagcagtgcgaggagctggtggacgtnaactgcagcgccgtgttggcttanttcctctgtgcccatgtacgngcccatttgcaccctggagttcctgcacgaccctatcaagccgtgagaagtncggtgtgncaagcgcgtgngaacgnacttggaagcccctcatgaagatgttacaaccacagttggggcccgnaagcctnggcctgcaacgancttgcctgttctnatgaaccgtgngtgttttgcanttnggt

SEQ ID NO: 10 - DNA, 342 bp, Homo sapiens
  misc_feature (5): n equals a, t, g, or c
ggcanaaanaanaagggcgctttctgtctgccggggtcgcagcgganaagggnagtgccatgtncctctccatcctggccatcgagcagtacgaggagctggtggacgtaaactgcagcgccgtgntggcttnctncctctgtgcccatgtacgcgncccatttgncaccctggaagttcctgcacgaccctatncaagccgtgaaagtncggtgtgccaagagcgngcaacggactganaagcccctgcatgaagntgtnacaaccacagntggcccgaaagcctggcctggaaagggttgcctgttntatgaaccntgggngtgttgnattttggccttg

SEQ ID NO: 11 - DNA, 497 bp, Homo sapiens
  misc_feature (10): n equals a, t, g, or c
ggcagagggnagcaggaggctttccctttggtttggggggattactacgactggtgcgcccaaaccagccagtcccaagaagaacattaaaactaggagtgcccagaagagaacaaacccgaaaagagtgtgagctaactagtttccaaagcggagacttccgacttccttacaggatgaggctgggcattgcctgggaacagcctatgttaaggccatgtgccccttgccctaacaactcactgcagtgctcttcatagacacatcttgcagcattttttttaaggctatgctttcantttttttttgtaagccatcacaagncatagtggttaggttttgccctttgggttacagaaggttgagttnaaagntgggtggaaaaagggttgattggcaatggnattccagagttaaccgtngtggcatactctnaggaagngtaggggnaataatggntngttaacaatttcggaccnnattnggtgcattgtnaa

SEQ ID NO: 12 - DNA, 27 bp, Artificial sequence
  Contains an Nco I restriction site
gactccatgggcgtgcgcggcgcgccc

SEQ ID NO: 13 - DNA, 30 bp, Artificial sequence
  Contains a Bam HI restriction site
gactggatcccactcttttcgggtttgttc

SEQ ID NO: 14 - DNA, 74 bp, Artificial sequence
  Contains a Bam HI restriction enzyme site, and an efficient signal for initiation of translation in eukaryotic cells (Kozak, M., J. Mol. Biol. 196947-950 (1987))
gactggatccgccatcatggtttgcggatcacgtggaggtatgctactgcttccacgcgtccgctccatcctag

SEQ ID NO: 15 - DNA, 29 bp, Artificial sequence
  Contains an Asp 718 restriction site
gactggtaccggaagtcggaagtctccgc

SEQ ID NO: 16 - DNA, 30 bp, Artificial sequence
  Contains an Xho I restriction site
gactctcgagcactcttttcgggtttgttc

SEQ ID NO: 17 - DNA, 733 bp, Homo sapiens
gggatccggagcccaaatcttctgacaaaactcacacatgcccaccgtgcccagcacctgaattcgagggtgcaccgtcagtcttcctcttccccccaaaacccaaggacaccctcatgatctcccggactcctgaggtcacatgcgtggtggtggacgtaagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaacccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccgcgactctagaggat

SEQ ID NO: 18 - protein, 5 aa, Homo sapiens
  MISC_FEATURE (3)..(3): Xaa is any amino acid
WSXWS

SEQ ID NO: 19 - DNA, 86 bp, Artificial sequence
  Contains four tandem copies of the GAS binding site found in the IRF1 promoter, 18 bp of sequence complementary to the SV40 early promoter sequence, and an Xho I restriction site
gcgcctcgagatttccccgaaatctagatttccccgaaatgatttccccgaaatgatttccccgaaatatctgccatctcaattag

SEQ ID NO: 20 - DNA, 27 bp, Artificial sequence
  Contains a HindIII restriction site
gcggcaagctttttgcaaagcctaggc

SEQ ID NO: 21 - DNA, 271 bp, Artificial sequence
  Synthetic GAS containing promoter element insert
ctcgagatttccccgaaatctagatttccccgaaatgatttccccgaaatgatttccccgaaatatctgccatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctt

SEQ ID NO: 22 - DNA, 32 bp, Artificial sequence
  5′ primer for EGR/SEAP reporter construct
gcgctcgagggatgacagcgatagaaccccgg

SEQ ID NO: 23 - DNA, 31 bp, Artificial sequence
  3′ primer for EGR/SEAP reporter construct
gcgaagcttcgcgactccccggatccgcctc

SEQ ID NO: 24 - DNA, 12 bp, Homo sapiens
ggggactttccc

SEQ ID NO: 25 - DNA, 73 bp, Artificial sequence
  Contains four tandem copies of the NF-kB binding site, 18 bp of sequence complementary to the 5′ end of the SV40 early promoter sequence, and an Xho I restriction site
gcggcctcgaggggactttcccggggactttccggggactttccgggactttccatcctgccatctcaattag

SEQ ID NO: 26 - DNA, 27 bp, Artificial sequence
  Contains a HindIII restriction site
gcggcaagctttttgcaaagcctaggc

SEQ ID NO: 27 - DNA, 256 bp, Artificial sequence
  NF-kB/SV40 vector insert
ctcgaggggactttcccggggactttccggggactttccgggactttccatctgccatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctt

(19) United States
(12) Patent Application Publication
(10) Pub. No.: US 2004/0038275 A1
(43) Pub. Date: Feb. 26, 2004

(54) Human frezzled-like protein

(75) Inventors: Henrik S. Olsen, Gaithersburg, MD (US); Steven M. Ruben, Brookeville, MD (US)

Correspondence Address: HUMAN GENOME SCIENCES INC, 9410 KEY WEST AVENUE, ROCKVILLE, MD, 20850

(73) Assignee: Human Genome Sciences, Inc., Rockville, MD

(21) Appl. No.: 10/610,917

(22) Filed: Jul. 2, 2003

Related U.S. Application Data
(63) Continuation of application No. 09/610,650, filed on Jun. 30, 2000, now abandoned.
Application No. 09/610,650 is a continuation of application No. 09/132,315, filed on Aug. 11, 1998, now abandoned.
(60) Provisional application No. 60/055,715, filed on Aug. 12, 1997.
(60) Provisional application No. 60/081,438, filed on Apr. 10, 1998.

(51) Int. Cl. C12Q 1/68; C07H 21/04; C07K 14/705

(52) U.S. Cl. 435/6; 435/69.1; 435/320.1; 435/325; 530/350; 536/23.5

(57) ABSTRACT

The present invention relates to a novel HFLP protein which is a member of the frizzled family. In particular, isolated nucleic acid molecules are provided encoding the human HFLP protein. HFLP polypeptides are also provided as are vectors, host cells and recombinant methods for producing the same. The invention further relates to screening methods for identifying agonists and antagonists of HFLP activity. Also provided are diagnostic methods for detecting cell growth and differentiation-related disorders and therapeutic methods for treating cell growth and differentiation-related disorders.

This application is a continuation of U.S. application Ser. No. 09/610,650, filed Jun. 30, 2000, which is a continuation of U.S. application Ser. No. 09/132,315, filed Aug. 11, 1998, which claims benefit under 35 U.S.C. § 119(e) of U.S. Provisional Application No. 60/081,438, filed Apr. 10, 1998, and No. 60/055,715, filed Aug. 12, 1997. U.S. Provisional Application Nos. 60/081,438 and 60/055,715 are each incorporated by reference herein in their entirety.

### FIELD OF THE INVENTION

The present invention relates to a novel human gene encoding a polypeptide which is member of the HFLP family. More specifically, isolated nucleic acid molecules are provided encoding a human polypeptide named human frezzled-like protein, hereinafter referred to as “HFLP”. Frezzled polypeptides are also provided, as are vectors, host cells and recombinant methods for producing the same. Also provided are diagnostic methods for detecting disorders related to cell growth and differentiation, and therapeutic methods for treating such disorders. The invention further relates to screening methods for identifying agonists and antagonists of HFLP activity.

### BACKGROUND OF THE INVENTION

A number of human genes have recently been identified as homologs of genes originally identified in organisms important in the study of development including Drosophila melanogaster, Caenorhabditis elegans, and Xenopus laevis. Interestingly, although the primary functional role of the original genes often relates to the regulation of development of those organisms, expression of the novel human homologs is also detectable in adult cells and tissues. Thus, it is likely that many genes which play an important role in embryonic development may continue to function throughout the lifespan of the organism.

The novel gene Frzb was recently identified as a factor important in the embryonic X. laevis developmental processes (Wang, S., et al., Cell 88:757-766 (1997); Moon, R. T., et al., Cell 88:725-728 (1997)). Frzb was initially isolated in an effort to identify signaling molecules involved in the early developmental process of skeletal patterning. Several genetic loci have been identified which elicit profound effects in the polarity and relative positioning of epidermal cells during the early stages of hair and bristle development in D. melanogaster, a well-studied system for studying skeletal patterning. One of the most well-known loci involved in these processes is frizzled. The Frizzled protein is a seven transmembrane receptor protein which is intimately involved in the cellular response to a presumably diffusible tissue polarity signal and in the intercellular transmission of that signal along the developing proximal/distal wing axis (Vinson, C. R. and Adler, P. N. Nature 329:549-551 (1987); Vinson, C. R., et al., Nature 338:263-264 (1989)). Although rat and human homologs of frizzled-1 and frizzled-2 have been identified and are expressed in a variety of tissues including kidney, liver, heart, uterus, and ovary (Chan, S. D. H., et al., J. Biol. Chem. 267:25202-25207 (1992); Zhao, Z., et al., Genomics 27:370-373 (1995)), at least six additional frizzled homologs have been identified recently which exhibit a more restricted expression pattern (Wang, Y., et al., J. Biol. Chem. 271:4468-4476 (1996)).

Although it contains a conserved frizzled-like domain, Frzb does not appear to be a seven transmembrane receptor, and, in fact, appears to be a secreted molecule. Recently, it was determined that Frzb may influence embryonic X. laevis developmental processes by interfering with the signal transduction pathways of the Wnt family of signalling proteins (Wang, S., et al., Cell 88:757-766 (1997); Moon, R. T., et al., Cell 88:725-728 (1997)). The wnts are a family of secreted proteins which appear to function in intercellular communication by interacting with the frizzled family of seven transmembrane receptors (Bhanot, P., et al., Nature 382:225-230 (1996); Yang-Snyder, J., et al., Curr. Biol. 6:1302-1306 (1996)). Studies by Leyns and colleagues (Cell 88:747-756 (1997)) and Wang and coworkers (Wang, S., et al., Cell 88:757-766 (1997)) demonstrate that Frzb protein binds to Wnt protein through a domain related at the sequence level to the Wnt-binding domain of the Frizzled protein. Such protein-protein interactions block the normal interactions of the Wnts to their receptors, and, in turn, add a level of complexity to the regulatory role of the Wnts in development.

The studies by Leyns and colleagues (Cell 88:747-756 (1997)) and Wang and coworkers (Wang, S., et al., Cell 88:757-766 (1997)), mentioned above, also determined that Frzb protein is expressed in the Spemann Organizer of the X. laevis embryo. The Spemann Organizer of the developing embyro is an area from which an increasingly large number of secreted factors implicated in both limb and axial patterning, including noggin, follistatin, chrodin, and nodal, have been identified. Secreted regulatory factors such as these have been found to play critical roles in the regulation of cell growth and differentiation, not only in development, but at various other points during the lifespan of the organism.

Thus, there is a need for polypeptides that function as regulators of cell growth and differentiation, since disturbances of such regulation may be involved in disorders relating to abnormal regulation of cell growth and differentiation, cancer, tissue regeneration, and wound healing. Therefore, there is a need for identification and characterization of such human polypeptides which can play a role in detecting, preventing, ameliorating or correcting such disorders.

### SUMMARY OF THE INVENTION

The present invention provides isolated nucleic acid molecules comprising a polynucleotide encoding at least a portion of the HFLP polypeptide having the complete amino acid sequence shown in SEQ ID NO:2 or the complete amino acid sequence encoded by the cDNA clone deposited as plasmid DNA as ATCC Deposit Number 209140 on Jul. 9, 1997. The nucleotide sequence determined by sequencing the deposited HFLP clone, which is shown in FIGS. 1A and 1B (SEQ ID NO:1) and in FIGS. 2A and 2B (SEQ ID NO:3), contains an open reading frame encoding a complete polypeptide of 368 amino acid residues (FIGS. 2A and 2B), including an initiating methionine residue at nucleotide positions 78-80 (FIGS. 2A and 2B), and a predicted molecular weight of about 42,060 Daltons. Nucleic acid molecules of the invention include those encoding the complete amino acid sequence excepting the N-terminal methionine shown in SEQ ID NO:2 and SEQ ID NO:4, or the complete amino acid sequence excepting the N-terminal methionine encoded by the cDNA clone in ATCC Deposit Number 209140, which molecule also can encode additional amino acids fused to the N-terminus of the HFLP amino acid sequence.

The HFLP protein of the present invention shares sequence homology with the translation product of the bovine mRNA for frezzled (Frzb; FIG. 3; SEQ ID NO:5), including the predicted frizzled domain of about 120 amino acids. Bovine Frzb is thought to be important in the regulation of cell growth and differentiation in the developing embryo and possibly throughout the life of the organism. The homology between bovine Frzb and HFLP indicates that HFLP may also be involved in the regulation of cellular growth and differentiation.

The encoded polypeptide has a predicted leader sequence of at least 19 amino acids underlined in FIG. 1A; and the amino acid sequence of the predicted mature HFLP protein is also shown in FIGS. 1A and 1B, as amino acid residues 20 to 347, and as residues 1-328 in SEQ ID NO:2. Further, upon obtaining and sequencing the clone identified as HDTBS70S01, following additional PCR-screening for a full-length HFLP clone, it is the case that the full-length encoded polypeptide has a predicted leader sequence of approximately 43 amino acids underlined in FIG. 2A; and the amino acid sequence of the predicted mature HFLP protein is shown in FIGS. 2A and 2B, as amino acid residues 44 to 368, and as residues 1-325 in SEQ ID NO:4.

Thus, one aspect of the invention provides an isolated nucleic acid molecule comprising a polynucleotide comprising a nucleotide sequence selected from the group consisting of: (a) a nucleotide sequence encoding the HFLP polypeptide having the complete amino acid sequence in SEQ ID NO:4 (i.e., positions −43 to 325 of SEQ ID NO:4); (b) a nucleotide sequence encoding the HFLP polypeptide having the complete amino acid sequence in SEQ ID NO:4 (i.e., positions −42 to 325 of SEQ ID NO:4), excluding the N-terminal methionine; (c) a nucleotide sequence encoding the predicted mature HFLP polypeptide having the amino acid sequence at positions 1 to 325 in SEQ ID NO:4; (d) a nucleotide sequence encoding the HFLP polypeptide having the complete amino acid sequence in SEQ ID NO:2 (i.e., positions −19 to 328 of SEQ ID NO:2) plus the N-terminal 12 amino acid signal peptide from the bovine Frzb molecule; (e) a nucleotide sequence encoding the HFLP polypeptide having the complete amino acid sequence in SEQ ID NO:2 (i.e., positions −19 to 328 of SEQ ID NO:2) plus the N-terminal 12 amino acid signal peptide from the bovine Frzb molecule, excluding the N-terminal methionine; (f) a nucleotide sequence encoding the HFLP polypeptide having the complete amino acid sequence in SEQ ID NO:2 (i.e., positions −19 to 328 of SEQ ID NO:2); (g) a nucleotide sequence encoding the predicted mature HFLP polypeptide having the amino acid sequence at positions 1 to 328 in SEQ ID NO:2; (h) a nucleotide sequence encoding the conserved frizzled domain of the HFLP polypeptide having the amino acid sequence in SEQ ID NO:2 (i.e., positions 6 to 126 of SEQ ID NO:2); (i) a nucleotide sequence encoding the HFLP polypeptide having the complete amino acid sequence encoded by the cDNA clone contained in ATCC Deposit No. 209140; (j) a nucleotide sequence encoding the mature HFLP polypeptide having the amino acid sequence encoded by the cDNA clone contained in ATCC Deposit No. 209140; (k) a nucleotide sequence encoding the frizzled domain of the HFLP polypeptide having the amino acid sequence encoded by the cDNA clone contained in ATCC Deposit No. 209140; and (l) a nucleotide sequence complementary to any of the nucleotide sequences in (a), (b), (c), (d), (e), (f), (g), (h), (i), (j), or (k), above.

Further embodiments of the invention include isolated nucleic acid molecules that comprise a polynucleotide having a nucleotide sequence at least 90% identical, and more preferably at least 95%, 96%, 97%, 98% or 99% identical, to any of the nucleotide sequences in (a), (b), (c), (d), (e), (f), (g), (h), (i), (j), (k), or (l), above, or a polynucleotide which hybridizes under stringent hybridization conditions to a polynucleotide in (a), (b), (c), (d), (e), (f), (g), (h), (i), (j), (k), or (l), above. This polynucleotide which hybridizes does not hybridize under stringent hybridization conditions to a polynucleotide having a nucleotide sequence consisting of only A residues or of only T residues. An additional nucleic acid embodiment of the invention relates to an isolated nucleic acid molecule comprising a polynucleotide which encodes the amino acid sequence of an epitope-bearing portion of a HFLP polypeptide having an amino acid sequence in (a), (b), (c), (d), (e), (f), (g), (h), (i), (j), or (k), above.

The present invention also relates to recombinant vectors, which include the isolated nucleic acid molecules of the present invention, and to host cells containing the recombinant vectors, as well as to methods of making such vectors and host cells and for using them for production of HFLP polypeptides or peptides by recombinant techniques.

The invention further provides an isolated HFLP polypeptide comprising an amino acid sequence selected from the group consisting of: (a) the amino acid sequence of the full-length HFLP polypeptide having the complete amino acid sequence shown in SEQ ID NO:4 (i.e., positions −43 to 325 of SEQ ID NO:4); (b) the amino acid sequence of the full-length HFLP polypeptide having the complete amino acid sequence shown in SEQ ID NO:4 (i.e., positions −42 to 325 of SEQ ID NO:4), excluding the N-terminal methionine; (c) the amino acid sequence of the mature HFLP polypeptide having the complete amino acid sequence in SEQ ID NO:4 (i.e., positions 1 to 325 of SEQ ID NO:4); (d) the amino acid sequence of the full-length HFLP polypeptide having the complete amino acid sequence shown in SEQ ID NO:2 (i.e., positions −19 to 328 of SEQ ID NO:2) plus the N-terminal 12 amino acid signal peptide from the bovine Frzb molecule; (e) the amino acid sequence of the full-length HFLP polypeptide having the complete amino acid sequence shown in SEQ ID NO:2 (i.e., positions −19 to 328 of SEQ ID NO:2) plus the N-terminal 12 amino acid signal peptide from the bovine Frzb molecule, excluding the N-terminal methionine; (f) the amino acid sequence of the HFLP polypeptide having the complete amino acid sequence in SEQ ID NO:2 (i.e., positions −19 to 328 of SEQ ID NO:2); (g) the amino acid sequence of the predicted mature HFLP polypeptide having the amino acid sequence at positions 1 to 328 in SEQ ID NO:2; (h) the amino acid sequence of the predicted frizzled domain of the HFLP polypeptide having the amino acid sequence in SEQ ID NO:2 (i.e., positions 6 to 126 of SEQ ID NO:2); (i) the amino acid sequence of the HFLP polypeptide having the complete amino acid sequence encoded by the cDNA clone contained in ATCC Deposit No. 209140; (j) the amino acid sequence of the mature HFLP polypeptide having the amino acid sequence encoded by the cDNA clone contained in ATCC Deposit No. 209140; and (k) the amino acid sequence of the frizzled domain of the HFLP polypeptide having the amino acid sequence encoded by the cDNA clone contained in ATCC Deposit No. 209140. The polypeptides of the present invention also include polypeptides having an amino acid sequence at least 80% identical, more preferably at least 90% identical, and still more preferably 95%, 96%, 97%, 98% or 99% identical to those described in (a), (b), (c), (d), (e), (f), (g), (h), (i), (j), or (k), above, as well as polypeptides having an amino acid sequence with at least 90% similarity, and more preferably at least 95% similarity, to those above.

An additional embodiment of this aspect of the invention relates to a peptide or polypeptide which comprises the amino acid sequence of an epitope-bearing portion of a HFLP polypeptide having an amino acid sequence described in (a), (b), (c), (d), (e), (f), (g), (h), (i), (j), or (k), above. Peptides or polypeptides having the amino acid sequence of an epitope-bearing portion of a HFLP polypeptide of the invention include portions of such polypeptides with at least six or seven, preferably at least nine, and more preferably at least about 30 amino acids to about 50 amino acids, although epitope-bearing polypeptides of any length up to and including the entire amino acid sequence of a polypeptide of the invention described above also are included in the invention.

In another embodiment, the invention provides an isolated antibody that binds specifically to a HFLP polypeptide having an amino acid sequence described in (a), (b), (c), (d), (e), (f), (g), (h), (i), (j), or (k), above. The invention further provides methods for isolating antibodies that bind specifically to a HFLP polypeptide having an amino acid sequence as described herein. Such antibodies are useful diagnostically or therapeutically as described below.

The invention also provides for pharmaceutical compositions comprising HFLP polypeptides, particularly human HFLP polypeptides, which may be employed, for instance, to treat cellular growth and differentiation disorders. Methods of treating individuals in need of HFLP polypeptides are also provided.

The invention further provides compositions comprising a HFLP polynucleotide or an HFLP polypeptide for administration to cells in vitro, to cells ex vivo and to cells in vivo, or to a multicellular organism. In certain particularly preferred embodiments of this aspect of the invention, the compositions comprise a HFLP polynucleotide for expression of a HFLP polypeptide in a host organism for treatment of disease. Particularly preferred in this regard is expression in a human patient for treatment of a dysfunction associated with aberrant endogenous activity of a HFLP

In another aspect, a screening assay for agonists and antagonists is provided which involves determining the effect a candidate compound has on HFLP binding to a ligand. In particular, the method involves contacting the ligand with a HFLP polypeptide and a candidate compound and determining whether HFLP polypeptide binding to the ligand is increased or decreased due to the presence of the candidate compound. In this assay, an increase in binding of HFLP over the standard binding indicates that the candidate compound is an agonist of HFLP binding activity and a decrease in HFLP binding compared to the standard indicates that the compound is an antagonist of HFLP binding activity.

It has been discovered that HFLP is expressed not only in cells obtained from a Hodgkin's Lymphoma, but also in adipose tissue, fetal lung, atrophic endometrium, synovial fibroblasts, synovial hypoxia, chondrosarcoma, pancreatic tumor, ovary, osteoclastoma, menijioma, hepatocellular tumor, IL-1- and TNF-stimulated synovial cells, osteoblasts, uterus, striatum depression, chronic synovitis, substantia nigra, spinal cord, testes, placenta, and whole 8 week old human embryo. Therefore, nucleic acids of the invention are useful as hybridization probes for differential identification of the tissue(s) or cell type(s) present in a biological sample. Similarly, polypeptides and antibodies directed to those polypeptides are useful to provide immunological probes for differential identification of the tissue(s) or cell type(s). In addition, for a number of disorders of the above tissues or cells, particularly with regard to cell growth and differentiation, significantly higher or lower levels of HFLP gene expression may be detected in certain tissues (e.g., cancerous and wounded tissues) or bodily fluids (e.g., serum, plasma, urine, synovial fluid or spinal fluid) taken from an individual having such a disorder, relative to a “standard” HFLP gene expression level, i.e., the HFLP expression level in healthy tissue from an individual not having the cell growth and differentiation disorder. Thus, the invention provides a diagnostic method useful during diagnosis of such a disorder, which involves: (a) assaying HFLP gene expression level in cells or body fluid of an individual; (b) comparing the HFLP gene expression level with a standard HFLP gene expression level, whereby an increase or decrease in the assayed HFLP gene expression level compared to the standard expression level is indicative of disorder in cell growth and differentiation.

An additional aspect of the invention is related to a method for treating an individual in need of an increased level of HFLP activity in the body comprising administering to such an individual a composition comprising a therapeutically effective amount of an isolated HFLP polypeptide of the invention or an agonist thereof.

A still further aspect of the invention is related to a method for treating an individual in need of a decreased level of HFLP activity in the body comprising, administering to such an individual a composition comprising a therapeutically effective amount of an HFLP antagonist. Preferred antagonists for use in the present invention are HFLP-specific antibodies.

### BRIEF DESCRIPTION OF THE FIGURES

FIGS. 1A and 1B show the nucleotide sequence (SEQ ID NO:1) and deduced amino acid sequence (SEQ ID NO:2) of HFLP. A potential N-terminal signal sequence is underlined and the conserved frizzled domain is double-underlined.

FIGS. 2A and 2B show the nucleotide sequence (SEQ ID NO:3) and deduced amino acid sequence (SEQ ID NO:4) of a fill-length clone HFLP designated HDTBS70S01. A potential N-terminal signal sequence is underlined and the conserved frizzled domain is double-underlined.

FIG. 3 shows the regions of identity between the amino acid sequences of the HFLP protein (SEQ ID NO:4) and translation product of the bovine mRNA for FRZB (SEQ ID NO:5), determined by the computer program Bestfit (Wisconsin Sequence Analysis Package, Version 8 for Unix, Genetics Computer Group, University Research Park, 575 Science Drive, Madison, Wis. 53711) using the default parameters.

FIG. 4 shows an analysis of the HFLP amino acid sequence (SEQ ID NO:4). Alpha, beta, turn and coil regions; hydrophilicity and hydrophobicity; amphipathic regions; flexible regions; antigenic index and surface probability are shown. In the “Antigenic Index or Jameson-Wolf” graph, the positive peaks indicate locations of the highly antigenic regions of the HFLP protein, i.e., regions from which epitope-bearing peptides of the invention can be obtained.

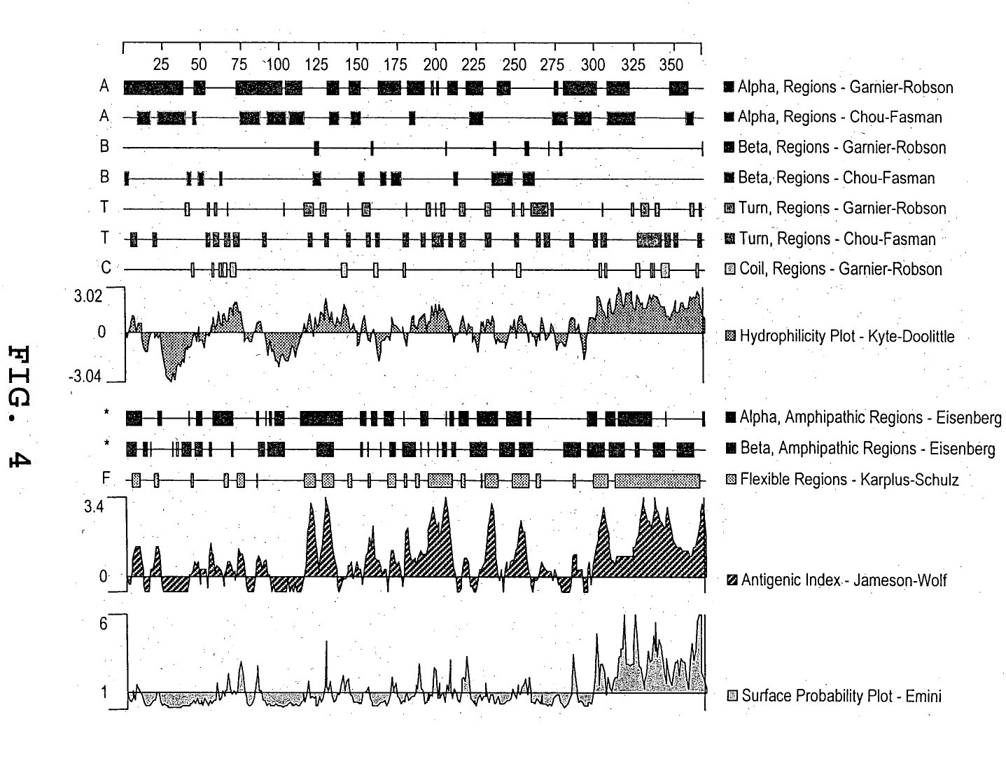

The data presented in FIG. 4 are also represented in tabular form in Table I. The columns are labeled with the headings “Res”, “Position”, and Roman Numerals I-XIII. The column headings refer to the following features of the amino acid sequence presented in FIG. 4 and Table I: “Res”: amino acid residue of SEQ ID NO:2; “Position”: position of the corresponding residue within SEQ ID NO:2; I: Alpha, Regions—Garnier-Robson; II: Alpha, Regions—Chou-Fasman; III: Beta, Regions—Garnier-Robson; IV: Beta, Regions—Chou-Fasman; V: Turn, Regions—Garnier-Robson; VI: Turn, Regions—Chou-Fasman; VII: Coil, Regions—Gamier-Robson; VIII: Hydrophilicity Plot—Kyte-Doolittle; IX: Alpha, Amphipathic Regions—Eisenberg; X: Beta, Amphipathic Regions—Eisenberg; XI: Flexible Regions—Karplus-Schulz; XIII: Antigenic Index—Jameson-Wolf; and XII: Surface Probability Plot—Emini.

### DETAILED DESCRIPTION

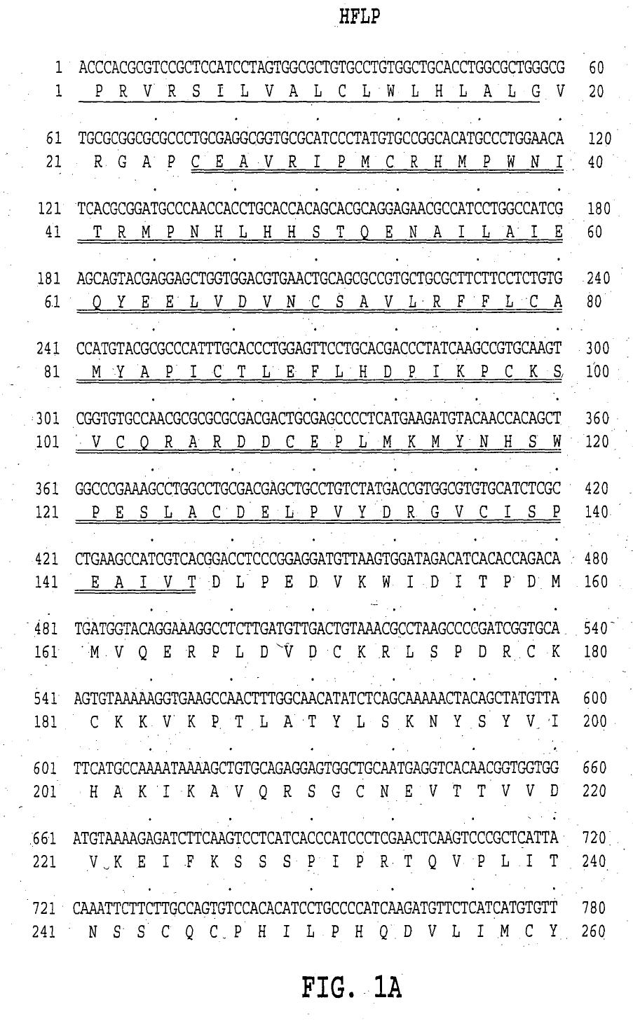

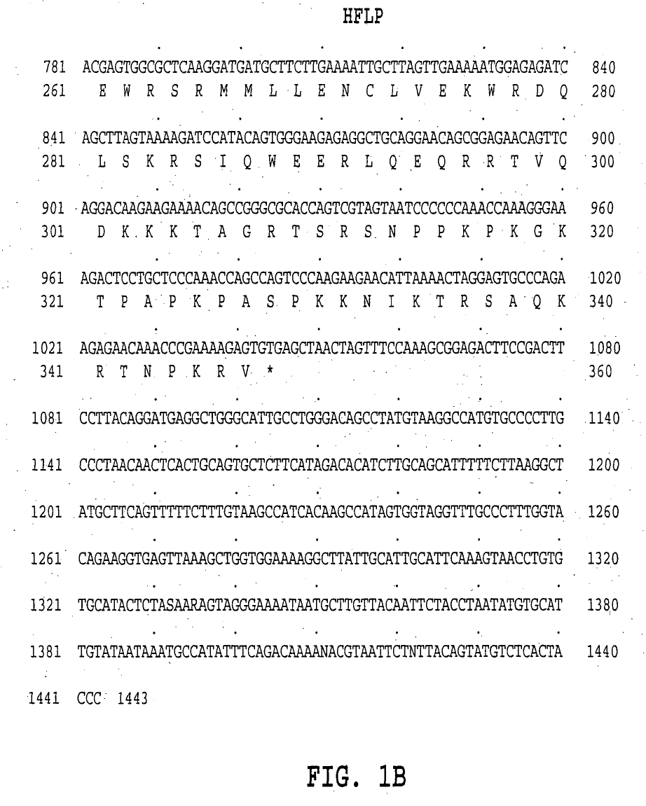

The present invention provides isolated nucleic acid molecules comprising a polynucleotide encoding an HFLP polypeptide having the amino acid sequence shown in SEQ ID NO:2, which was determined by sequencing a cloned cDNA. The nucleotide sequence shown in FIGS. 1A and 1B (SEQ ID NO:1) was obtained by sequencing the HDTBS70 clone, which was deposited on Jul. 9, 1997, at the American Type Culture Collection, 10801 University Boulevard, Manassas, Va. 20110-2209, and given accession number ATCC 209140. The deposited clone is contained in the pBluescript SK(−) plasmid (Stratagene, La Jolla, Calif.).

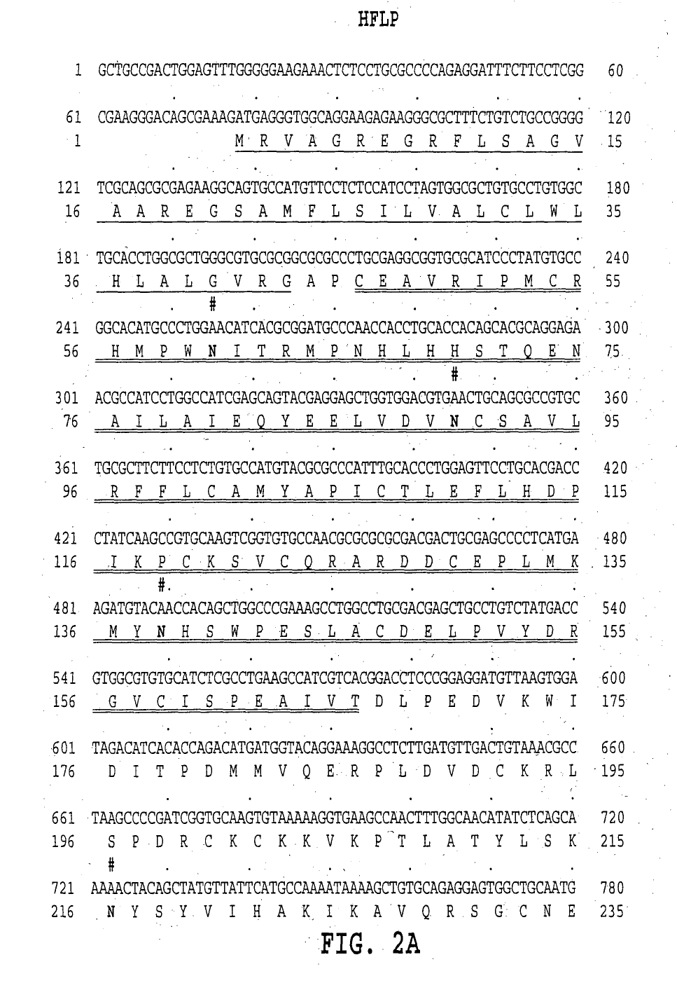

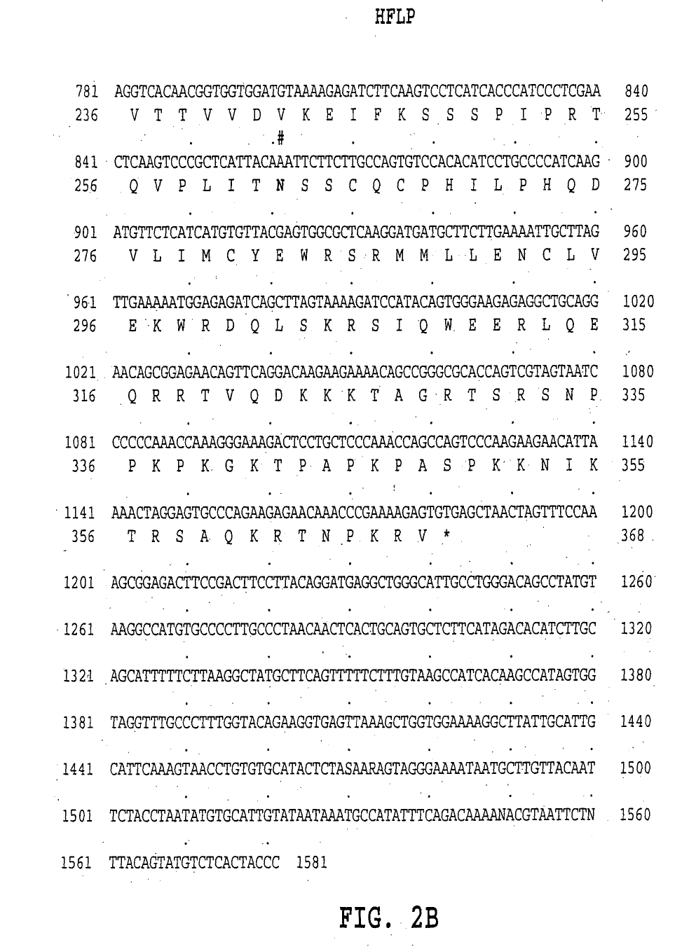

A cDNA clone which is believed to be a full-length clone of HFLP was subsequently obtained by PCR-screening and is designated HDTBS70S01. The nucleotide and amino acid sequences of the full-length HDTBS70S01 HFLP clone are provided in FIGS. 2A and 2B as SEQ ID NO:3 and SEQ ID NO:4, respectively.

With regard to resolving apparent differences in the overlapping nucleotide sequence between clones HDTBS70 and HDTBS70S01, the sequence of the deposited cDNA clone HDTBS70 is preferred.

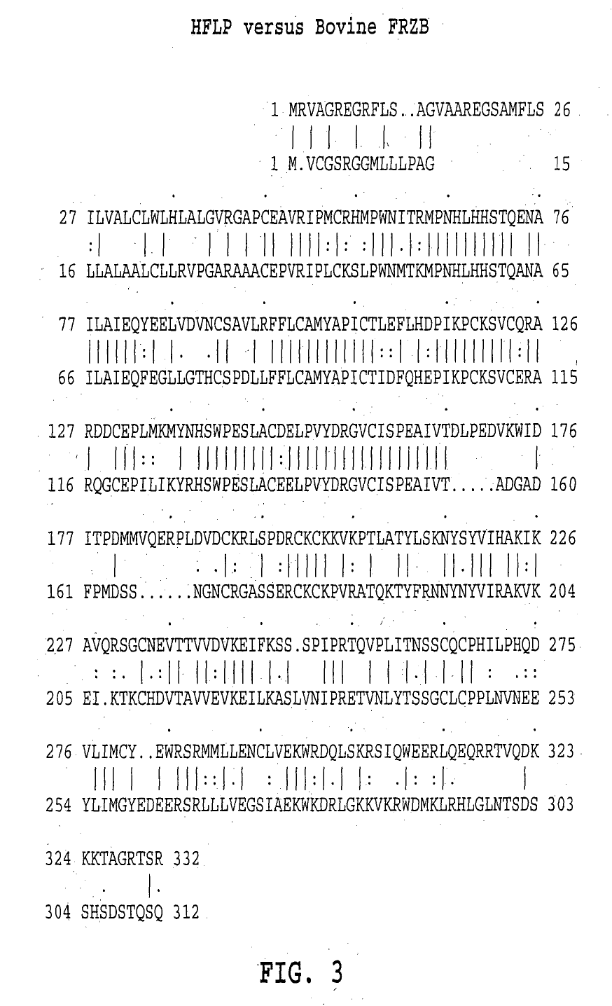

The HFLP protein of the present invention shares sequence homology with the translation product of the bovine mRNA for Frzb (FIG. 3; SEQ ID NO:5). Frzb is thought to be an important modulatory factor in the developmental process. Frzb contains a frizzled homology domain which binds to the Wnt family of proteins. The interaction of the secreted Frzb molecule with a member of the Wnt family of proteins prevents binding of the Wnt protein to membrane bound receptor molecules which also contain frizzled homology domains, and, as a result, prevent stimulation of the intracellular component of the Wnt-related signal transduction pathway.

### Nucleic Acid Molecules

Unless otherwise indicated, all nucleotide sequences determined by sequencing a DNA molecule herein were determined using an automated DNA sequencer (such as the Model 373 from Applied Biosystems, Inc., Foster City, Calif.), and all amino acid sequences of polypeptides encoded by DNA molecules determined herein were predicted by translation of a DNA sequence determined as above. Therefore, as is known in the art for any DNA sequence determined by this automated approach, any nucleotide sequence determined herein may contain some errors. Nucleotide sequences determined by automation are typically at least about 90% identical, more typically at least about 95% to at least about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion.

By “nucleotide sequence” of a nucleic acid molecule or polynucleotide is intended, for a DNA molecule or polynucleotide, a sequence of deoxyribonucleotides, and for an RNA molecule or polynucleotide, the corresponding sequence of ribonucleotides (A, G, C and U), where each thymidine deoxyribonucleotide (T) in the specified deoxyribonucleotide sequence is replaced by the ribonucleotide uridine (U).

Using the information provided herein, such as the nucleotide sequence in FIGS. 1A and 1B (SEQ ID NO:1) or 2A and 2B (SEQ ID NO:3), a nucleic acid molecule of the present invention encoding a HFLP polypeptide may be obtained using standard cloning and screening procedures, such as those for cloning cDNAs using mRNA as starting material. Illustrative of the invention, the nucleic acid molecule described in FIGS. 1A and 1B (SEQ ID NO:1) was discovered in a cDNA library derived from Hodgkin's Lymphoma.

Additional clones of the same gene were also identified in cDNA libraries from the following cells and tissues: adipose tissue, fetal lung, atrophic endometrium, synovial fibroblasts, synovial hypoxia, chondrosarcoma, pancreatic tumor, ovary, osteoclastoma, menijioma, hepatocellular tumor, IL-1- and TNF-stimulated synovial cells, osteoblasts, uterus, striatum depression, chronic synovitis, substantia nigra, spinal cord, testes, placenta, and whole 8 week old human embryo.

The determined nucleotide sequence of the HFLP cDNA of FIGS. 1A and 1B (SEQ ID NO:1) contains an open reading frame encoding a protein of 347 amino acid residues, with an initiation codon at nucleotide positions 3-5 of the nucleotide sequence in FIG. 1A (SEQ ID NO:1), and a deduced molecular weight of about 40.0 kDa.

The determined nucleotide sequence of the full-length HFLP cDNA of FIGS. 2A and 2B (SEQ ID NO:3) contains an open reading frame encoding a protein of 368 amino acid residues, with an initiation codon at nucleotide positions 78-80 of the nucleotide sequence in FIG. 2B (SEQ ID NO:3), and a deduced molecular weight of about 42.1 kDa. The amino acid sequence of the HFLP protein shown in SEQ ID NO:4 is about 54% identical to bovine mRNA for FRZB (FIG. 3; Hoang, B., et al., J. Biol. Chem. 271:26131-26137 (1996); GenBank Accession No. U24164).

The open reading frame of the HFLP gene shares sequence homology with the translation product of the bovine mRNA for Frzb (FIG. 3; SEQ ID NO:5), including the predicted frizzled domain of about 120 amino acids. Bovine Frzb is thought to be important in the regulation of cellular growth and differentiation during development and possibly throughout the life of the organism. The homology between bovine Frzb and HFLP indicates that HFLP may also be involved in the regulation of cellular growth and differentiation.

As one of ordinary skill would appreciate, due to the possibilities of sequencing errors discussed above, the actual complete HFLP polypeptide encoded by the “full-length” cDNA, which comprises about 368 amino acids, may be somewhat longer or shorter. More generally, the actual open reading frame may be anywhere in the range of ±20 amino acids, more likely in the range of ±10 amino acids, of that predicted from the methionine codon at the N-terminus shown in FIG. 2A (SEQ ID NO:3). It will further be appreciated that, depending on the analytical criteria used for identifying various functional domains, the exact “address” of the frizzled domain of the HFLP polypeptide may differ slightly from the predicted positions above.

Leader and Mature Sequences

The amino acid sequence of the complete HFLP protein includes a leader sequence and a mature protein, as shown in SEQ ID NO:2 and SEQ ID NO:4. More in particular, the present invention provides nucleic acid molecules encoding a mature form of the HFLP protein. Thus, according to the signal hypothesis, once export of the growing protein chain across the rough endoplasmic reticulum has been initiated, proteins secreted by mammalian cells have a signal or secretory leader sequence which is cleaved from the complete polypeptide to produce a secreted “mature” form of the protein. Most mammalian cells and even insect cells cleave secreted proteins with the same specificity. However, in some cases, cleavage of a secreted protein is not entirely uniform, which results in two or more mature species of the protein. Further, it has long been known that the cleavage specificity of a secreted protein is ultimately determined by the primary structure of the complete protein, that is, it is inherent in the amino acid sequence of the polypeptide. Therefore, the present invention provides a nucleotide sequence encoding the mature HFLP polypeptide having the amino acid sequence encoded by the cDNA clone contained in the host identified as ATCC Deposit No. 209140. By the “mature HFLP polypeptide having the amino acid sequence encoded by the cDNA clone in ATCC Deposit No. 209140” is meant the mature form(s) of the HFLP protein produced by expression in a mammalian cell (e.g., COS cells, as described below) of the complete open reading frame encoded by the human DNA sequence of the clone contained in the deposited vector. Since cleavage specificity may vary according to host cell and expression conditions, the “mature HFLP polypeptide having the amino acid sequence encoded by the cDNA clone in ATCC Deposit No. 209140” also identifies any mature species which comprises ±5 amino acids from the predicted cleavage point of the deposited clone shown in FIGS. 1A and 1B (SEQ ID NO:2). As such, the mature HFLP polypeptide also comprises amino acids −5 to 328, −4 to 328, −3 to 328, −2 to 328, −1 to 328, 1 to 328, 2 to 328, 3 to 328, 4 to 328, and 5 to 328 of the polypeptide encoded by the deposited clone, either alone or in any combination thereof.

In addition, methods for predicting whether a protein has a secretory leader as well as the cleavage point for that leader sequence are available. For instance, the method of McGeoch (Virus Res. 3:271-286 (1985)) uses the information from a short N-terminal charged region and a subsequent uncharged region of the complete (uncleaved) protein. The method of von Heinje (Nucleic Acids Res. 14:4683-4690 (1986)) uses the information from the residues surrounding the cleavage site, typically residues −13 to +2 where +1 indicates the amino terminus of the mature protein. The accuracy of predicting the cleavage points of known mammalian secretory proteins for each of these methods is in the range of 75-80% (von Heinje, supra). However, the two methods do not always produce the same predicted cleavage point(s) for a given protein.

In the present case, the deduced amino acid sequence of the complete HFLP polypeptide was analyzed by a computer program “PSORT”, available from Dr. Kenta Nakai of the Institute for Chemical Research, Kyoto University (Nakai, K. and Kanehisa, M. Genomics 14:897-911 (1992)), which is an expert system for predicting the cellular location of a protein based on the amino acid sequence. As part of this computational prediction of localization, the methods of McGeoch and von Heinje are incorporated. Thus, the computation analysis above predicted a single N-terminal cleavage site within the complete amino acid sequence shown in SEQ ID NO:2. Further, the computation analysis above also predicted a single N-terminal cleavage site within the complete amino acid sequence shown in SEQ ID NO:4.

As indicated, nucleic acid molecules of the present invention may be in the form of RNA, such as mRNA, or in the form of DNA, including, for instance, cDNA and genomic DNA obtained by cloning or produced synthetically. The DNA may be double-stranded or single-stranded. Single-stranded DNA or RNA may be the coding strand, also known as the sense strand, or it may be the non-coding strand, also referred to as the anti-sense strand.

By “isolated” nucleic acid molecule(s) is intended a nucleic acid molecule, DNA or RNA, which has been removed from its native environment For example, recombinant DNA molecules contained in a vector are considered isolated for the purposes of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. Isolated nucleic acid molecules according to the present invention further include such molecules produced synthetically.

Isolated nucleic acid molecules of the present invention include DNA molecules comprising an open reading frame (ORF) beginning presumably in frame with a proline codon at positions 3-5 of the nucleotide sequence shown in FIG. 1A (SEQ ID NO:1). Also included are DNA molecules comprising the coding sequence for the predicted mature HFLP protein shown at positions 1-328 of SEQ ID NO:2.

Isolated nucleic acid molecules of the present invention also include DNA molecules comprising an ORF beginning with a methionine codon at positions 78-80 of the nucleotide sequence shown in FIG. 2A (SEQ ID NO:3). Also included are DNA molecules comprising the coding sequence for the predicted mature HFLP protein shown at positions 1-325 of SEQ ID NO:4.

In addition, isolated nucleic acid molecules of the invention include DNA molecules which comprise a sequence substantially different from those described above but which, due to the degeneracy of the genetic code, still encode the HFLP protein. Of course, the genetic code and species-specific codon preferences are well known in the art. Thus, it would be routine for one skilled in the art to generate the degenerate variants described above, for instance, to optimize codon expression for a particular host (e.g., change codons in the human mRNA to those preferred by a bacterial host such as E. coli).

In another aspect, the invention provides isolated nucleic acid molecules encoding the HFLP polypeptide having an amino acid sequence encoded by the cDNA clone contained in the plasmid deposited as ATCC Deposit No. 209140 on Jul. 9, 1997.

Preferably, this nucleic acid molecule will encode the mature polypeptide encoded by the above-described deposited cDNA clone.

The invention further provides an isolated nucleic acid molecule having the nucleotide sequences shown in FIGS. 1A and 1B (SEQ ID NO:1), 2A and 2B (SEQ ID NO:3), or the nucleotide sequence of the HFLP cDNA contained in the above-described deposited clone, or a nucleic acid molecule having a sequence complementary to one of the above sequences. Such isolated molecules, particularly DNA molecules, are useful as probes for gene mapping, by in situ hybridization with chromosomes, and for detecting expression of the HFLP gene in human tissue, for instance, by Northern blot analysis.

The present invention is further directed to nucleic acid molecules encoding portions of the nucleotide sequences described herein as well as to fragments of the isolated nucleic acid molecules described herein. In particular, the invention provides a polynucleotide having a nucleotide sequence representing the portion of SEQ ID NO:1 which consists of positions 1-1046 of SEQ ID NO:1 and a polynucleotide representing the portion of SEQ ID NO:3 which consists of positions 1-1581 of SEQ ID NO:3.

In addition, the invention provides nucleic acid molecules having nucleotide sequences related to extensive portions of SEQ ID NO:1 and SEQ ID NO:3 which have been determined from the following related cDNA clones: HUNAG89R (SEQ ID NO:6), HCDAR36R (SEQ ID NO:7), HCDDT20R (SEQ ID NO:8), HCDDN09R (SEQ ID NO:9), HCDBV59R (SEQ ID NO:10), and HCDDC90R (SEQ ID NO:11).

Further, the invention includes a polynucleotide comprising any portion of at least about 30 nucleotides, preferably at least about 50 nucleotides, of SEQ ID NO:1 from residue 600-1144. More preferably, the invention includes a polynucleotide comprising nucleotide residues 250-1144, 500-1144, 600-1144, 750-1144, 1000-1144, 250-1000, 500-1000, 600-1000, 750-1000, 250-750, 500-750, 600-750, 250-600, and 500-600.

More generally, by a fragment of an isolated nucleic acid molecule having the nucleotide sequence of the deposited cDNA or the nucleotide sequence shown in FIGS. 1A and 1B (SEQ ID NO:1) or FIGS. 2A and 2B (SEQ ID NO:3) is intended fragments at least about 15 nt, and more preferably at least about 20 nt, still more preferably at least about 30 nt, and even more preferably, at least about 40 nt in length which are useful as diagnostic probes and primers as discussed herein. Of course, larger fragments 50-300 nt in length are also useful according to the present invention as are fragments corresponding to most, if not all, of the nucleotide sequence of the deposited cDNA or as shown in FIG. 1A and 1B (SEQ ID NO:1) or 2A and 2B (SEQ ID NO:3). By a fragment at least 20 nt in length, for example, is intended fragments which include 20 or more contiguous bases from the nucleotide sequence of the deposited cDNA or the nucleotide sequence as shown in FIG. 1A and 1B (SEQ ID NO:1) or 2A and 2B (SEQ ID NO:3). Preferred nucleic acid fragments of the present invention include nucleic acid molecules encoding epitope-bearing portions of the HFLP polypeptide as identified in FIGS. 4 and 5, and described in more detail below.

Also preferred in this aspect of the invention are fragments characterized by structural or functional attributes of HFLP. Preferred embodiments of the invention in this regard include fragments that comprise alpha-helix and alpha-helix forming regions (“alpha-regions”), beta-sheet and beta-sheet forming regions (“beta-regions”), turn and turn-forming regions (“turn-regions”), coil and coil-forming regions (“coil-regions”), hydrophilic regions, hydrophobic regions, alpha amphipathic regions, beta amphipathic regions, flexible regions, surface-forming regions and high antigenic index regions of HFLP.

Certain preferred regions in these regards are set out in FIG. 4, but may, as shown in Table I, also be represented or identified by using a tabular representation of the data presented in FIG. 4. The DNA*STAR computer algorithm used to generate FIG. 4 (set on the original default parameters) was used to present the data in FIG. 4 in a tabular format (Table I). The tabular format of the data shown in Table I may be used to easily determine specific boundaries of a preferred region.

The above-mentioned preferred regions set out in FIG. 4 and Table I include, but are not limited to, regions of the aforementioned types identified by analysis of the amino acid sequence set out in FIGS. 1A and 1B. As set out in FIG. 4 and Table I, such preferred regions include Garnier-Robson alpha-regions, beta-regions, turn-regions, and coil-regions, Chou-Fasman alpha-regions, beta-regions, and coil-regions, Kyte-Doolittle hydrophilic regions and hydrophobic regions, Eisenberg alpha- and beta-amphipathic regions, Karplus-Schulz flexible regions, Emini surface-forming regions and Jameson-Wolf regions of high antigenic index.

TABLE I

| Res | Position | I | II | III | IV | V | VI | VII | VIII | IX | X | XI | XII | XIII |
|---|---|---|---|---|---|---|---|---|---|---|---|---|---|---|
| Pro | 1 | . | . | . | B | . | . | C | 0.80 | . | * | . | 0.95 | 1.65 |
| Arg | 2 | . | . | . | B | T | . | . | 0.30 | * | * | . | 1.15 | 1.73 |
| Val | 3 | A | . | . | B | . | . | . | −0.12 | . | * | . | 0.30 | 0.95 |
| Arg | 4 | A | . | . | B | . | . | . | −0.59 | . | * | . | 0.30 | 0.51 |
| Ser | 5 | . | . | B | B | . | . | . | −0.79 | * | * | . | −0.30 | 0.19 |
| Ile | 6 | . | A | B | . | . | . | . | −1.39 | * | * | . | −0.60 | 0.26 |
| Leu | 7 | . | A | B | . | . | . | . | −2.17 | . | * | . | −0.60 | 0.11 |
| Val | 8 | A | A | . | . | . | . | . | −2.12 | * | . | . | −0.60 | 0.04 |
| Ala | 9 | A | A | . | . | . | . | . | −2.52 | * | . | . | −0.60 | 0.05 |
| Leu | 10 | A | A | . | . | . | . | . | −3.03 | . | . | . | −0.60 | 0.07 |
| Cys | 11 | A | A | . | . | . | . | . | −2.18 | . | * | . | −0.60 | 0.07 |
| Leu | 12 | A | A | . | . | . | . | . | −2.18 | . | . | . | −0.60 | 0.10 |
| Trp | 13 | A | A | . | . | . | . | . | −1.91 | . | * | . | −0.60 | 0.10 |
| Leu | 14 | A | A | . | . | . | . | . | −2.13 | . | . | . | −0.60 | 0.19 |
| His | 15 | A | A | . | . | . | . | . | −1.67 | . | * | . | −0.60 | 0.19 |
| Leu | 16 | A | A | . | . | . | . | . | −1.86 | . | * | . | −0.60 | 0.17 |
| Ala | 17 | A | A | . | . | . | . | . | −0.93 | . | * | . | −0.60 | 0.16 |
| Leu | 18 | . | A | . | . | T | . | . | −0.99 | . | * | . | −0.20 | 0.23 |
| Gly | 19 | . | A | . | . | T | . | . | −0.77 | . | * | . | 0.10 | 0.27 |
| Val | 20 | . | . | . | B | T | . | . | −0.94 | * | * | . | 0.10 | 0.27 |
| Arg | 21 | . | . | . | B | T | . | . | −0.80 | . | * | F | 0.25 | 0.51 |
| Gly | 22 | . | . | . | B | . | . | C | −0.21 | . | . | F | 0.65 | 0.28 |
| Ala | 23 | . | A | . | . | . | . | C | 0.01 | . | * | . | 0.50 | 0.64 |
| Pro | 24 | A | A | . | . | . | . | . | −0.50 | * | * | . | 0.60 | 0.33 |
| Cys | 25 | A | A | . | . | . | . | . | 0.47 | * | * | . | −0.30 | 0.25 |
| Glu | 26 | A | A | . | . | . | . | . | −0.53 | * | * | . | 0.30 | 0.48 |
| Ala | 27 | A | . | . | B | . | . | . | −0.40 | * | * | . | 0.30 | 0.22 |
| Val | 28 | A | . | . | B | . | . | . | −0.41 | * | * | . | 0.30 | 0.63 |
| Arg | 29 | A | . | . | B | . | . | . | −0.87 | . | . | . | 0.30 | 0.36 |
| Ile | 30 | A | . | . | B | . | . | . | −0.09 | . | . | . | −0.50 | 0.19 |
| Pro | 31 | A | . | . | . | . | T | . | −0.12 | . | . | . | 0.90 | 0.51 |
| Met | 32 | . | . | . | . | T | T | . | −0.13 | . | * | . | 1.40 | 0.35 |
| Cys | 33 | . | . | . | . | T | T | . | 0.51 | . | * | . | 0.60 | 0.50 |
| Arg | 34 | . | . | . | . | T | T | . | 0.11 | . | . | . | 1.00 | 0.50 |
| His | 35 | . | . | . | . | . | . | C | 1.00 | * | . | . | 0.20 | 0.53 |
| Met | 36 | . | . | . | . | . | T | C | 0.32 | * | . | . | 0.45 | 1.58 |
| Pro | 37 | . | . | . | . | T | T | . | 0.61 | * | . | . | 0.40 | 0.57 |
| Trp | 38 | . | . | . | . | T | T | . | 1.39 | * | . | . | 0.30 | 0.60 |
| Asn | 39 | . | . | . | . | . | T | C | 0.68 | * | . | . | 0.15 | 1.19 |
| Ile | 40 | . | . | . | B | . | . | C | 0.50 | * | . | . | −0.40 | 0.76 |
| Thr | 41 | . | . | . | B | T | . | . | 1.10 | * | . | F | 0.10 | 1.12 |
| Arg | 42 | . | . | . | B | . | . | C | 1.28 | * | . | F | 0.20 | 1.12 |
| Met | 43 | . | . | . | . | . | T | C | 0.76 | * | . | F | 0.60 | 2.17 |
| Pro | 44 | . | . | . | . | . | T | C | 0.72 | * | . | F | 0.60 | 1.24 |
| Asn | 45 | . | . | . | . | T | T | . | 1.58 | * | . | . | 0.50 | 0.86 |
| His | 46 | . | . | . | . | . | T | C | 1.59 | * | * | . | 0.15 | 1.18 |
| Leu | 47 | . | . | . | . | . | . | C | 1.17 | * | * | . | 0.25 | 1.03 |
| His | 48 | . | . | . | . | . | . | C | 1.77 | * | . | . | −0.20 | 0.92 |
| His | 49 | . | . | . | . | . | T | C | 1.98 | . | . | . | 0.15 | 1.17 |
| Ser | 50 | . | . | . | . | . | T | C | 1.98 | . | . | F | 1.20 | 2.46 |
| Thr | 51 | A | . | . | . | . | T | . | 1.42 | . | . | F | 1.00 | 2.91 |
| Gln | 52 | A | . | . | . | . | T | . | 1.34 | . | . | F | 1.00 | 2.16 |
| Glu | 53 | A | A | . | . | . | . | . | 0.57 | . | . | F | 0.60 | 1.13 |
| Asn | 54 | A | A | . | . | . | . | . | 0.01 | . | . | F | −0.15 | 0.65 |
| Ala | 55 | A | A | . | . | . | . | . | −0.58 | . | . | . | −0.30 | 0.38 |
| Ile | 56 | A | A | . | . | . | . | . | −0.27 | . | . | . | −0.60 | 0.15 |
| Leu | 57 | A | A | . | . | . | . | . | −0.27 | . | . | . | −0.60 | 0.16 |
| Ala | 58 | A | A | . | . | . | . | . | −0.51 | . | . | . | −0.60 | 0.28 |
| Ile | 59 | A | A | . | . | . | . | . | −0.51 | . | . | . | −0.60 | 0.63 |
| Glu | 60 | A | A | . | . | . | . | . | 0.08 | . | . | . | −0.15 | 1.32 |
| Gln | 61 | A | A | . | . | . | . | . | 0.16 | . | . | . | 0.75 | 2.26 |
| Tyr | 62 | A | A | . | . | . | . | . | 0.11 | * | . | F | 0.60 | 2.66 |
| Glu | 63 | A | A | . | . | . | . | . | 0.70 | * | . | F | 0.90 | 1.14 |
| Glu | 64 | A | A | . | . | . | . | . | 0.73 | * | * | . | 0.75 | 1.10 |
| Leu | 65 | A | A | . | . | . | . | . | 0.73 | . | * | . | 0.30 | 0.52 |
| Val | 66 | A | A | . | . | . | . | . | 0.07 | . | * | . | 0.60 | 0.48 |
| Asp | 67 | A | . | . | . | . | T | . | 0.01 | . | * | . | 0.70 | 0.15 |
| Val | 68 | A | . | . | . | . | T | . | −0.58 | * | . | . | 0.10 | 0.24 |
| Asn | 69 | A | . | . | . | . | T | . | −1.43 | . | * | . | 0.10 | 0.33 |
| Cys | 70 | A | . | . | . | . | T | . | −1.43 | * | * | . | 0.10 | 0.15 |
| Ser | 71 | A | A | . | . | . | . | . | −0.47 | * | * | . | −0.60 | 0.16 |
| Ala | 72 | A | A | . | . | . | . | . | −1.17 | * | * | . | −0.30 | 0.20 |
| Val | 73 | A | A | . | . | . | . | . | −1.01 | . | * | . | −0.60 | 0.32 |
| Leu | 74 | A | A | . | . | . | . | . | −1.82 | * | * | . | −0.60 | 0.21 |
| Arg | 75 | A | A | . | . | . | . | . | −1.82 | * | * | . | −0.60 | 0.17 |
| Phe | 76 | A | A | . | . | . | . | . | −2.11 | * | * | . | −0.60 | 0.12 |
| Phe | 77 | A | A | . | . | . | . | . | −2.12 | * | * | . | −0.60 | 0.15 |
| Leu | 78 | A | A | . | . | . | . | . | −1.51 | * | * | . | −0.60 | 0.08 |
| Cys | 79 | A | A | . | . | . | . | . | −1.29 | * | * | . | −0.60 | 0.14 |
| Ala | 80 | A | A | . | . | . | . | . | −1.61 | * | * | . | −0.60 | 0.16 |
| Met | 81 | . | A | . | . | T | . | . | −1.80 | . | . | . | −0.20 | 0.30 |
| Tyr | 82 | A | A | . | . | . | . | . | −1.77 | . | . | . | −0.60 | 0.39 |
| Ala | 83 | A | . | . | . | . | . | . | −1.27 | . | . | . | −0.40 | 0.21 |
| Pro | 84 | A | . | . | . | . | . | . | −1.41 | . | . | . | −0.40 | 0.30 |
| Ile | 85 | A | A | . | . | . | . | . | −0.82 | . | . | . | −0.60 | 0.16 |
| Cys | 86 | A | A | . | . | . | . | . | −0.92 | . | . | . | −0.30 | 0.27 |
| Thr | 87 | A | A | . | . | . | . | . | −1.49 | . | . | . | −0.60 | 0.15 |
| Leu | 88 | A | A | . | . | . | . | . | −0.93 | . | . | . | −0.60 | 0.18 |
| Glu | 89 | A | A | . | . | . | . | . | −0.72 | . | . | . | −0.60 | 0.46 |
| Phe | 90 | A | A | . | . | . | . | . | −0.04 | * | . | . | −0.30 | 0.53 |
| Leu | 91 | A | A | . | . | . | . | . | −0.27 | * | . | . | −0.30 | 1.00 |
| His | 92 | A | A | . | . | . | . | . | 0.09 | * | . | . | 0.01 | 0.40 |
| Asp | 93 | A | A | . | . | . | . | . | 0.69 | * | . | F | 0.47 | 0.93 |
| Pro | 94 | . | A | . | . | T | . | . | 0.02 | * | . | F | 1.93 | 1.75 |
| Ile | 95 | . | . | . | . | T | . | . | 0.77 | * | . | F | 2.29 | 0.69 |
| Lys | 96 | . | . | . | . | T | T | . | 1.28 | * | . | F | 3.10 | 0.82 |
| Pro | 97 | . | . | . | . | T | T | . | 0.46 | * | . | F | 2.79 | 0.71 |
| Cys | 98 | . | . | . | . | T | T | . | −0.21 | * | . | F | 2.18 | 0.76 |
| Lys | 99 | . | . | . | . | T | T | . | −0.00 | * | . | F | 1.87 | 0.20 |
| Ser | 100 | . | . | . | B | T | . | . | 1.00 | * | . | F | 1.16 | 0.23 |
| Val | 101 | . | . | B | B | . | . | . | 0.37 | * | * | . | 0.30 | 0.83 |
| Cys | 102 | . | . | B | B | . | . | . | 0.69 | * | * | . | 0.94 | 0.42 |
| Gln | 103 | . | . | B | B | . | . | . | 1.36 | * | * | . | 1.28 | 0.61 |
| Arg | 104 | . | . | . | B | T | . | . | 1.31 | * | * | F | 2.32 | 1.38 |
| Ala | 105 | . | . | . | . | T | . | . | 0.94 | * | * | F | 2.86 | 4.29 |
| Arg | 106 | . | . | . | . | T | T | . | 1.80 | * | * | F | 3.40 | 1.33 |
| Asp | 107 | . | . | . | . | T | T | . | 2.26 | * | * | F | 3.06 | 1.17 |
| Asp | 108 | . | . | . | . | T | T | . | 1.44 | * | * | F | 2.72 | 1.80 |
| Cys | 109 | A | . | . | . | . | T | . | 0.73 | * | * | F | 1.83 | 0.76 |
| Glu | 110 | A | A | . | . | . | . | . | 1.37 | * | * | F | 1.09 | 0.45 |
| Pro | 111 | A | A | . | . | . | . | . | 0.66 | * | * | F | 0.75 | 0.54 |
| Leu | 112 | A | A | . | . | . | . | . | 0.41 | * | . | . | 0.30 | 0.99 |
| Met | 113 | A | A | . | . | . | . | . | 0.41 | * | . | . | −0.30 | 0.90 |
| Lys | 114 | A | A | . | . | . | . | . | 1.04 | * | . | . | −0.60 | 0.93 |
| Met | 115 | A | A | . | . | . | . | . | 0.74 | * | . | . | −0.45 | 1.54 |
| Tyr | 116 | A | A | . | . | . | . | . | 0.67 | * | . | . | −0.15 | 2.08 |
| Asn | 117 | . | . | . | . | . | . | C | 1.27 | * | . | . | −0.05 | 1.10 |
| His | 118 | . | . | . | . | . | . | C | 1.87 | . | . | . | −0.05 | 1.71 |
| Ser | 119 | . | . | . | . | . | . | C | 1.52 | . | . | . | 0.25 | 1.89 |
| Trp | 120 | . | . | . | . | . | T | C | 1.31 | . | . | . | 0.45 | 1.58 |
| Pro | 121 | . | . | . | . | . | T | C | 0.97 | . | . | F | 0.45 | 0.96 |
| Glu | 122 | . | . | . | . | T | T | . | 0.30 | . | . | F | 0.65 | 0.72 |
| Ser | 123 | A | . | . | . | . | T | . | 0.33 | . | . | F | −0.05 | 0.37 |
| Leu | 124 | A | A | . | . | . | . | . | 0.63 | . | . | . | 0.30 | 0.40 |
| Ala | 125 | A | A | . | . | . | . | . | 0.11 | . | . | . | 0.60 | 0.40 |
| Cys | 126 | A | A | . | . | . | . | . | 0.11 | . | . | . | 0.30 | 0.24 |
| Asp | 127 | A | A | . | . | . | . | . | −0.74 | . | . | . | 0.30 | 0.46 |
| Glu | 128 | A | A | . | . | . | . | . | −0.69 | . | . | . | 0.30 | 0.34 |
| Leu | 129 | A | A | . | B | . | . | . | 0.12 | * | * | . | −0.30 | 0.98 |
| Pro | 130 | A | A | . | B | . | . | . | 0.82 | * | * | . | 0.30 | 0.98 |
| Val | 131 | . | . | . | B | T | . | . | 1.14 | * | . | . | 0.85 | 1.11 |
| Tyr | 132 | . | . | . | B | T | . | . | 0.29 | * | . | . | 1.07 | 1.33 |
| Asp | 133 | . | . | . | . | T | T | . | −0.38 | . | * | F | 1.69 | 0.64 |
| Arg | 134 | . | . | . | . | T | T | . | −0.46 | . | . | F | 1.31 | 0.46 |
| Gly | 135 | . | . | . | . | T | T | . | −0.54 | . | . | . | 1.38 | 0.21 |
| Val | 136 | . | . | . | . | T | T | . | 0.10 | * | . | . | 2.20 | 0.17 |
| Cys | 137 | . | . | B | . | . | . | . | 0.34 | * | . | . | 0.78 | 0.13 |
| Ile | 138 | . | . | B | . | . | . | C | −0.24 | * | . | . | 0.76 | 0.23 |
| Ser | 139 | . | . | . | . | . | T | C | −1.24 | * | . | . | 0.74 | 0.31 |
| Pro | 140 | . | . | . | . | . | T | C | −1.76 | . | . | . | 0.52 | 0.41 |
| Glu | 141 | A | . | . | . | . | T | . | −1.21 | . | . | . | 0.10 | 0.43 |
| Ala | 142 | A | . | . | . | . | T | . | −0.54 | . | . | . | 0.10 | 0.47 |
| Ile | 143 | A | . | . | B | . | . | . | −0.47 | . | . | . | 0.30 | 0.50 |
| Val | 144 | A | . | . | B | . | . | . | −0.38 | * | . | . | 0.30 | 0.24 |
| Thr | 145 | A | . | . | B | . | . | . | −0.17 | * | . | . | −0.60 | 0.37 |
| Asp | 146 | A | . | . | B | . | . | . | −0.17 | * | . | F | 0.45 | 0.91 |
| Leu | 147 | A | . | . | . | . | . | . | −0.43 | * | * | F | 1.10 | 2.04 |
| Pro | 148 | A | . | . | . | . | . | . | 0.50 | * | * | F | 1.10 | 1.05 |
| Glu | 149 | A | . | . | . | . | . | . | 1.07 | * | * | F | 1.10 | 1.25 |
| Asp | 150 | A | . | . | B | . | . | . | 0.49 | * | * | F | 0.60 | 1.60 |
| Val | 151 | A | . | . | B | . | . | . | 0.49 | . | * | F | 0.45 | 0.73 |
| Lys | 152 | A | . | . | B | . | . | . | 0.41 | . | . | . | 0.60 | 0.70 |
| Trp | 153 | A | . | . | B | . | . | . | 0.31 | . | . | . | 0.30 | 0.29 |
| Ile | 154 | A | . | . | B | . | . | . | 0.10 | . | . | . | −0.39 | 0.57 |
| Asp | 155 | A | . | . | B | . | . | . | 0.10 | . | * | . | 0.12 | 0.44 |
| Ile | 156 | A | . | . | B | . | . | . | 0.36 | * | . | . | 0.33 | 0.70 |
| Thr | 157 | . | . | . | . | . | T | C | −0.29 | * | * | F | 1.89 | 0.99 |
| Pro | 158 | . | . | . | . | . | T | C | −0.86 | . | * | F | 2.10 | 0.59 |
| Asp | 159 | . | . | . | . | T | T | . | 0.03 | . | * | . | 1.04 | 0.62 |
| Met | 160 | A | . | . | . | . | T | . | 0.03 | . | * | . | 0.73 | 0.75 |
| Met | 161 | A | A | . | . | . | . | . | 1.03 | . | * | . | 0.72 | 0.83 |
| Val | 162 | A | A | . | . | . | . | . | 1.13 | . | * | . | 0.81 | 0.98 |
| Gln | 163 | A | A | . | . | . | . | . | 0.53 | . | * | . | 0.45 | 1.53 |
| Glu | 164 | A | A | . | . | . | . | . | 0.53 | . | * | F | 0.60 | 1.27 |
| Arg | 165 | A | A | . | . | . | . | . | 0.28 | . | . | F | 0.90 | 2.87 |
| Pro | 166 | A | . | . | . | . | . | . | 0.88 | . | . | F | 1.10 | 1.23 |
| Leu | 167 | A | . | . | . | . | . | . | 1.07 | * | * | . | 0.95 | 1.19 |
| Asp | 168 | A | . | . | . | . | T | . | 1.11 | * | . | . | 1.00 | 0.32 |
| Val | 169 | A | . | . | . | . | T | . | 1.22 | * | . | . | 1.00 | 0.42 |
| Asp | 170 | A | . | . | . | . | T | . | 0.30 | * | . | . | 1.30 | 1.00 |
| Cys | 171 | . | . | . | . | T | T | . | 0.21 | * | * | . | 2.00 | 0.49 |
| Lys | 172 | . | . | . | . | T | . | . | 0.81 | * | * | F | 2.25 | 0.89 |
| Arg | 173 | . | . | . | . | T | . | . | 0.81 | . | . | F | 2.55 | 0.82 |
| Leu | 174 | . | . | . | . | T | . | . | 1.78 | . | . | F | 3.00 | 2.56 |
| Ser | 175 | A | . | . | . | . | T | . | 1.11 | * | . | F | 2.50 | 2.51 |
| Pro | 176 | A | . | . | . | . | T | . | 1.82 | . | . | F | 2.05 | 0.69 |
| Asp | 177 | . | . | . | . | T | T | . | 1.11 | . | * | F | 2.30 | 1.67 |
| Arg | 178 | A | . | . | . | . | T | . | 1.04 | . | * | F | 1.79 | 0.67 |
| Cys | 179 | A | . | . | . | . | T | . | 1.90 | . | . | F | 1.83 | 0.86 |
| Lys | 180 | A | . | . | . | . | T | . | 1.34 | . | . | F | 2.32 | 1.03 |
| Cys | 181 | . | . | . | . | T | T | . | 1.60 | . | . | F | 2.91 | 0.39 |
| Lys | 182 | . | . | . | . | T | T | . | 1.39 | . | * | F | 3.40 | 1.46 |
| Lys | 183 | . | . | . | . | T | . | . | 0.97 | * | * | F | 2.86 | 1.13 |
| Val | 184 | . | . | B | . | . | . | . | 0.82 | . | . | F | 2.12 | 3.04 |
| Lys | 185 | . | . | B | . | . | T | . | 0.19 | . | . | F | 1.98 | 1.25 |
| Pro | 186 | A | . | . | . | . | T | . | 0.54 | * | . | F | 1.19 | 0.63 |
| Thr | 187 | A | . | . | . | . | T | . | 0.26 | * | * | F | 0.40 | 1.23 |
| Leu | 188 | A | . | . | . | . | T | . | −0.60 | * | * | . | 0.10 | 0.96 |
| Ala | 189 | A | . | . | B | . | . | . | −0.04 | . | * | . | −0.60 | 0.51 |
| Thr | 190 | A | . | . | B | . | . | . | −0.04 | . | . | . | −0.60 | 0.48 |
| Tyr | 191 | A | . | . | B | . | . | . | 0.17 | * | . | . | −0.45 | 1.16 |
| Leu | 192 | A | . | . | B | . | . | . | 0.23 | * | . | . | −0.15 | 1.84 |
| Ser | 193 | . | . | . | . | T | T | . | 0.74 | * | . | F | 0.50 | 2.00 |
| Lys | 194 | . | . | . | . | T | T | . | 1.09 | * | . | F | 0.80 | 1.71 |
| Asn | 195 | . | . | . | . | T | T | . | 0.54 | * | . | F | 0.80 | 3.25 |
| Tyr | 196 | . | . | . | . | T | T | . | −0.10 | * | . | . | 0.65 | 1.80 |
| Ser | 197 | A | . | . | . | . | . | . | 0.68 | * | . | . | −0.40 | 0.63 |
| Tyr | 198 | A | . | . | . | . | . | . | 0.39 | . | * | . | −0.40 | 0.53 |
| Val | 199 | A | A | . | . | . | . | . | 0.39 | . | * | . | −0.60 | 0.34 |
| Ile | 200 | A | A | . | . | . | . | . | −0.50 | . | * | . | −0.60 | 0.51 |
| His | 201 | A | A | . | . | . | . | . | −0.21 | . | * | . | −0.60 | 0.23 |
| Ala | 202 | A | A | . | . | . | . | . | −0.50 | . | * | . | −0.30 | 0.62 |
| Lys | 203 | A | A | . | . | . | . | . | −1.11 | * | * | . | 0.30 | 0.89 |
| Ile | 204 | A | A | . | . | . | . | . | −0.26 | * | * | . | 0.30 | 0.49 |
| Lys | 205 | A | A | . | . | . | . | . | 0.74 | * | * | F | 0.45 | 0.83 |
| Ala | 206 | A | A | . | . | . | . | . | 0.48 | * | * | . | 0.60 | 0.82 |
| Val | 207 | A | A | . | . | . | . | . | 0.72 | * | * | . | 0.76 | 1.56 |
| Gln | 208 | A | A | . | . | . | . | . | 0.01 | * | * | F | 1.37 | 0.77 |
| Arg | 209 | . | . | . | . | T | T | . | 0.90 | * | * | F | 2.18 | 0.41 |
| Ser | 210 | . | . | . | . | T | T | . | 0.86 | * | . | F | 2.49 | 0.89 |
| Gly | 211 | . | . | . | . | T | T | . | 0.59 | * | . | F | 3.10 | 0.89 |
| Cys | 212 | . | . | . | . | T | T | . | 1.13 | * | . | F | 2.79 | 0.34 |
| Asn | 213 | . | . | . | B | . | . | C | 0.82 | * | . | F | 1.58 | 0.36 |
| Glu | 214 | . | . | . | B | T | . | . | −0.14 | * | . | F | 0.87 | 0.53 |
| Val | 215 | . | . | B | B | . | . | . | −0.70 | * | . | F | 0.16 | 0.73 |
| Thr | 216 | . | . | B | B | . | . | . | −0.36 | * | . | F | −0.15 | 0.34 |
| Thr | 217 | A | . | . | B | . | . | . | −0.54 | . | * | . | 0.30 | 0.33 |
| Val | 218 | A | . | . | B | . | . | . | −0.50 | . | * | . | −0.60 | 0.33 |
| Val | 219 | A | . | . | B | . | . | . | −0.50 | . | * | . | 0.30 | 0.45 |
| Asp | 220 | A | . | . | B | . | . | . | −0.53 | . | * | . | 0.60 | 0.54 |
| Val | 221 | A | . | . | B | . | . | . | −0.92 | * | * | . | 0.30 | 0.51 |
| Lys | 222 | A | . | . | B | . | . | . | −0.57 | * | * | . | 0.30 | 0.60 |
| Glu | 223 | A | . | . | B | . | . | . | −0.01 | * | * | . | 0.60 | 0.72 |
| Ile | 224 | A | . | . | B | . | . | . | 0.54 | * | * | . | 0.75 | 1.29 |
| Phe | 225 | A | . | . | B | . | . | . | 0.24 | * | * | F | 0.75 | 0.87 |
| Lys | 226 | . | . | . | B | T | . | . | 0.89 | * | * | F | 1.09 | 0.67 |
| Ser | 227 | . | . | . | . | T | T | . | −0.04 | * | . | F | 1.28 | 1.48 |
| Ser | 228 | . | . | . | . | . | T | C | −0.26 | * | . | F | 1.32 | 1.20 |
| Ser | 229 | . | . | . | . | . | T | C | 0.74 | * | . | F | 2.01 | 0.92 |
| Pro | 230 | . | . | . | . | . | T | C | 1.13 | * | * | F | 2.40 | 1.35 |
| Ile | 231 | . | . | . | . | . | . | C | 1.09 | * | * | F | 1.96 | 1.46 |
| Pro | 232 | . | . | . | . | T | . | . | 0.53 | . | * | F | 1.92 | 1.88 |
| Arg | 233 | . | . | . | B | T | . | . | 0.62 | * | * | F | 0.73 | 0.90 |
| Thr | 234 | . | . | B | B | . | . | . | 0.11 | * | * | F | 0.24 | 1.99 |
| Gln | 235 | . | . | B | B | . | . | . | −0.57 | * | . | F | 0.00 | 1.06 |
| Val | 236 | . | . | B | B | . | . | . | 0.01 | * | * | . | −0.30 | 0.38 |
| Pro | 237 | . | . | B | B | . | . | . | 0.22 | * | * | . | −0.60 | 0.38 |
| Leu | 238 | . | . | . | B | T | . | . | −0.19 | . | * | . | −0.20 | 0.35 |
| Ile | 239 | . | . | . | B | T | . | . | −0.18 | . | . | . | −0.20 | 0.64 |
| Thr | 240 | . | . | . | B | T | . | . | −0.84 | . | . | F | −0.05 | 0.55 |
| Asn | 241 | . | . | . | . | T | T | . | 0.01 | . | . | F | 0.35 | 0.36 |
| Ser | 242 | . | . | . | . | T | T | . | −0.44 | . | . | F | 0.65 | 0.89 |
| Ser | 243 | . | . | . | . | T | T | . | 0.16 | . | . | F | 0.65 | 0.33 |
| Cys | 244 | . | . | . | . | T | T | . | 1.01 | . | . | . | 0.50 | 0.32 |
| Gln | 245 | . | . | . | . | T | . | . | 0.43 | . | . | . | 0.30 | 0.32 |
| Cys | 246 | . | . | . | . | T | T | . | −0.38 | . | . | . | 0.20 | 0.17 |
| Pro | 247 | . | . | . | . | T | T | . | −0.29 | . | . | . | 0.20 | 0.26 |
| His | 248 | . | . | . | . | T | T | . | −0.02 | . | . | . | 0.20 | 0.23 |
| Ile | 249 | . | . | B | . | . | T | . | 0.64 | . | . | . | −0.20 | 0.59 |
| Leu | 250 | . | . | . | . | . | . | C | 0.64 | . | . | . | −0.20 | 0.66 |
| Pro | 251 | . | . | . | . | T | . | . | 0.46 | . | . | . | 0.30 | 0.81 |
| His | 252 | . | A | . | . | T | . | . | −0.14 | . | . | . | 0.10 | 0.85 |
| Gln | 253 | A | A | . | . | . | . | . | −1.00 | . | . | . | −0.30 | 0.85 |
| Asp | 254 | A | A | . | . | . | . | . | −0.71 | . | . | . | −0.30 | 0.39 |
| Val | 255 | A | A | . | . | . | . | . | −0.57 | . | . | . | −0.60 | 0.28 |
| Leu | 256 | . | A | B | . | . | . | . | −0.60 | . | . | . | −0.60 | 0.09 |
| Ile | 257 | . | A | B | . | . | . | . | −0.57 | . | . | . | −0.60 | 0.08 |
| Met | 258 | . | A | B | . | . | . | . | −0.86 | . | * | . | −0.60 | 0.19 |
| Cys | 259 | A | A | . | . | . | . | . | −0.74 | . | * | . | −0.60 | 0.24 |
| Tyr | 260 | A | A | . | . | . | . | . | −0.19 | . | * | . | −0.60 | 0.68 |
| Glu | 261 | A | A | . | . | . | . | . | 0.73 | . | * | . | −0.30 | 0.92 |
| Trp | 262 | A | . | . | . | . | T | . | 1.02 | . | * | . | 0.85 | 3.36 |
| Arg | 263 | A | . | . | . | . | T | . | 1.02 | . | * | F | 1.00 | 2.12 |
| Ser | 264 | A | . | . | . | . | T | . | 0.88 | . | * | F | 1.00 | 1.21 |
| Arg | 265 | A | . | . | . | . | T | . | 0.31 | . | * | . | 0.10 | 0.95 |
| Met | 266 | A | A | . | . | . | . | . | 0.31 | . | * | . | 0.30 | 0.40 |
| Met | 267 | A | A | . | . | . | . | . | 0.60 | . | * | . | 0.30 | 0.52 |
| Leu | 268 | A | A | . | . | . | . | . | −0.18 | . | * | . | 0.30 | 0.43 |
| Leu | 269 | A | A | . | . | . | . | . | −0.69 | . | . | . | −0.60 | 0.23 |
| Glu | 270 | A | A | . | . | . | . | . | −1.66 | . | . | . | −0.60 | 0.19 |
| Asn | 271 | A | A | . | . | . | . | . | −1.06 | . | . | . | −0.60 | 0.17 |
| Cys | 272 | A | A | . | . | . | . | . | −0.41 | . | . | . | 0.30 | 0.36 |
| Leu | 273 | A | A | . | . | . | . | . | 0.11 | * | * | . | 0.60 | 0.42 |
| Val | 274 | A | A | . | . | . | . | . | 1.03 | * | . | . | −0.30 | 0.27 |
| Glu | 275 | A | A | . | . | . | . | . | 1.03 | * | * | . | 0.45 | 1.00 |
| Lys | 276 | A | A | . | . | . | . | . | 1.03 | * | * | F | 0.90 | 2.03 |
| Trp | 277 | A | . | . | . | . | T | . | 0.89 | * | * | F | 1.30 | 4.73 |
| Arg | 278 | A | . | . | . | . | T | . | 1.40 | * | * | F | 1.60 | 2.25 |
| Asp | 279 | A | . | . | . | . | T | . | 2.30 | * | * | F | 1.90 | 1.51 |
| Gln | 280 | A | . | . | . | . | T | . | 2.41 | . | * | F | 2.20 | 2.87 |
| Leu | 281 | . | . | . | . | . | . | C | 2.07 | . | * | F | 2.50 | 2.87 |
| Ser | 282 | . | . | . | . | . | T | C | 1.47 | . | * | F | 3.00 | 2.30 |
| Lys | 283 | . | . | . | . | T | T | . | 1.36 | . | * | F | 2.45 | 0.93 |
| Arg | 284 | . | . | . | . | . | T | C | 1.07 | * | . | F | 2.10 | 1.96 |
| Ser | 285 | . | . | . | . | . | T | C | 1.07 | * | . | F | 1.80 | 1.54 |
| Ile | 286 | . | A | . | . | . | . | C | 1.88 | * | * | . | 1.25 | 1.33 |
| Gln | 287 | A | A | . | . | . | . | . | 2.29 | * | * | . | 0.75 | 1.18 |
| Trp | 288 | A | A | . | . | . | . | . | 1.43 | * | * | . | 0.75 | 1.72 |
| Glu | 289 | A | A | . | . | . | . | . | 1.32 | * | * | . | 0.45 | 2.02 |
| Glu | 290 | A | A | . | . | . | . | . | 1.62 | * | * | F | 0.90 | 2.02 |
| Arg | 291 | A | A | . | . | . | . | . | 2.51 | . | * | F | 0.90 | 3.33 |
| Leu | 292 | A | A | . | . | . | . | . | 2.62 | * | * | F | 0.90 | 3.33 |
| Gln | 293 | A | A | . | . | . | . | . | 3.02 | * | * | F | 0.90 | 3.77 |
| Glu | 294 | A | A | . | . | . | . | . | 2.71 | * | * | F | 0.90 | 3.77 |
| Gln | 295 | A | A | . | . | . | . | . | 1.86 | * | * | F | 0.90 | 6.59 |
| Arg | 296 | A | A | . | . | . | . | . | 1.74 | * | * | F | 0.90 | 2.83 |
| Arg | 297 | A | A | . | . | . | . | . | 2.56 | * | . | F | 0.90 | 2.83 |
| Thr | 298 | A | A | . | . | . | . | . | 2.60 | * | . | F | 0.90 | 2.72 |
| Val | 299 | A | A | . | . | . | . | . | 2.64 | * | . | F | 0.90 | 2.78 |
| Gln | 300 | A | A | . | . | . | . | . | 2.69 | * | . | F | 0.90 | 2.84 |
| Asp | 301 | . | A | . | . | T | . | . | 2.27 | * | . | F | 1.30 | 3.94 |
| Lys | 302 | . | A | . | . | T | . | . | 1.57 | * | . | F | 1.30 | 7.65 |
| Lys | 303 | . | A | . | . | T | . | . | 1.53 | * | * | F | 1.64 | 4.46 |
| Lys | 304 | . | A | . | . | . | . | C | 2.50 | * | * | F | 1.78 | 2.65 |
| Thr | 305 | . | . | . | . | . | T | C | 2.19 | * | * | F | 2.52 | 2.59 |
| Ala | 306 | . | . | . | . | . | T | C | 1.89 | * | . | F | 2.86 | 1.87 |
| Gly | 307 | . | . | . | . | T | T | . | 1.96 | * | . | F | 3.40 | 1.25 |
| Arg | 308 | . | . | . | . | T | T | . | 1.61 | * | . | F | 3.06 | 1.70 |
| Thr | 309 | . | . | . | . | T | T | . | 1.57 | * | . | F | 2.72 | 2.26 |
| Ser | 310 | . | . | . | . | T | T | . | 1.67 | * | . | F | 2.38 | 3.67 |
| Arg | 311 | . | . | . | . | T | T | . | 2.04 | * | . | F | 2.38 | 2.89 |
| Ser | 312 | . | . | . | . | T | T | . | 2.43 | * | . | F | 2.08 | 3.10 |
| Asn | 313 | . | . | . | . | . | T | C | 2.11 | * | . | F | 2.52 | 4.63 |
| Pro | 314 | . | . | . | . | . | T | C | 2.47 | . | * | F | 2.86 | 3.65 |
| Pro | 315 | . | . | . | . | T | T | . | 2.42 | . | * | F | 3.40 | 5.45 |
| Lys | 316 | . | . | . | . | . | T | C | 2.36 | . | * | F | 2.86 | 3.35 |
| Pro | 317 | . | . | . | . | T | T | . | 2.34 | . | * | F | 2.72 | 4.34 |
| Lys | 318 | . | . | . | . | T | T | . | 2.13 | . | * | F | 2.38 | 4.05 |
| Gly | 319 | . | . | . | . | T | T | . | 1.76 | . | * | F | 2.34 | 3.13 |
| Lys | 320 | . | . | . | . | . | T | C | 1.76 | . | * | F | 2.10 | 2.05 |
| Thr | 321 | . | . | . | . | . | . | C | 1.76 | . | * | F | 2.20 | 1.58 |
| Pro | 322 | . | . | . | . | . | . | C | 1.76 | * | * | F | 2.50 | 3.20 |
| Ala | 323 | . | . | . | . | . | T | C | 1.12 | * | . | F | 3.00 | 2.47 |
| Pro | 324 | . | . | . | . | . | T | C | 1.17 | . | . | F | 2.40 | 1.73 |
| Lys | 325 | . | . | . | . | . | T | C | 0.91 | . | . | F | 2.10 | 1.50 |
| Pro | 326 | A | . | . | . | . | T | . | 1.27 | . | . | F | 1.60 | 2.29 |
| Ala | 327 | A | . | . | . | . | . | . | 1.52 | . | . | F | 1.40 | 2.97 |
| Ser | 328 | A | . | . | . | . | T | . | 2.11 | . | . | F | 1.30 | 2.97 |
| Pro | 329 | A | . | . | . | . | T | . | 1.43 | . | * | F | 1.30 | 3.09 |
| Lys | 330 | A | . | . | . | . | T | . | 1.43 | . | * | F | 1.30 | 2.14 |
| Lys | 331 | A | . | . | . | . | T | . | 1.33 | . | * | F | 1.30 | 3.20 |
| Asn | 332 | A | . | . | . | . | . | . | 2.03 | . | * | F | 1.10 | 2.98 |
| Ile | 333 | A | . | . | . | . | . | . | 2.03 | . | * | F | 1.10 | 2.92 |
| Lys | 334 | A | . | . | . | . | . | . | 1.66 | . | * | F | 1.10 | 1.96 |
| Thr | 335 | A | . | . | . | . | . | . | 1.61 | . | * | F | 1.10 | 1.23 |
| Arg | 336 | A | A | . | . | . | . | . | 1.61 | . | * | F | 0.90 | 3.04 |
| Ser | 337 | A | A | . | . | . | . | . | 1.72 | . | * | F | 0.90 | 3.04 |
| Ala | 338 | A | A | . | . | . | . | . | 2.30 | . | * | F | 0.90 | 4.12 |
| Gln | 339 | . | A | . | . | T | . | . | 2.26 | . | * | F | 1.30 | 3.04 |
| Lys | 340 | . | A | . | . | T | . | . | 2.36 | . | * | F | 1.30 | 3.65 |
| Arg | 341 | . | A | . | . | T | . | . | 2.29 | . | . | F | 1.61 | 5.58 |
| Thr | 342 | . | . | . | . | . | . | C | 2.70 | . | . | F | 1.92 | 6.44 |
| Asn | 343 | . | . | . | . | . | T | C | 2.43 | . | . | F | 2.43 | 6.31 |
| Pro | 344 | . | . | . | . | . | T | C | 2.04 | . | . | F | 2.74 | 2.39 |
| Lys | 345 | . | . | . | . | T | T | . | 1.61 | . | . | . | 3.10 | 2.12 |
| Arg | 346 | . | . | . | . | T | T | . | 1.11 | * | . | . | 2.79 | 1.68 |
| Val | 347 | . | . | B | . | . | . | . | 1.03 | * | . | . | 1.88 | 1.39 |

One of skill in the art would recognize that the amino acid sequence of SEQ ID NO:4 can also be analyzed by the DNA*STAR computer program to generate data similar to those presented as FIG. 4 and Table I. Such an analysis would also include, but not be limited to, such preferred regions as Garnier-Robson alpha-regions, beta-regions, turn-regions, and coil-regions, Chou-Fasman alpha-regions, beta-regions, and coil-regions, Kyte-Doolittle hydrophilic regions and hydrophobic regions, Eisenberg alpha- and beta-amphipathic regions, Karplus-Schulz flexible regions, Emini surface-forming regions and Jameson-Wolf regions of high antigenic index. Moreover, although many preferred regions in these regards would be set forth in a graphic representation such as FIG. 4, they may also be represented or identified by using a tabular representation of the same data such as that shown in Table I. The DNA*STAR computer algorithm used to generate such FIG. 4 (set on the original default parameters) will easily present the data in a tabular format such as that shown in Table I. A tabular format of the data in a figure such as FIG. 4 may then be used to easily determine specific boundaries of a preferred region.

Among highly preferred fragments in this regard are those that comprise reigons of HFLP that combine several structural features, such as several of the features set out above.

In another aspect, the invention provides an isolated nucleic acid molecule comprising a polynucleotide which hybridizes under stringent hybridization conditions to a portion of the polynucleotide in a nucleic acid molecule of the invention described above, for instance, the cDNA clone contained in ATCC Deposit No. 209140. By “stringent hybridization conditions” is intended overnight incubation at 42° C. in a solution comprising: 50% formamide, 5×SSC (750 mM NaCl, 75 mM trisodium citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at about 65° C.

By a polynucleotide which hybridizes to a “portion” of a polynucleotide is intended a polynucleotide (either DNA or RNA) hybridizing to at least about 15 nucleotides (nt), and more preferably at least about 20 nt, still more preferably at least about 30 nt, and even more preferably about 30-70 (e.g., 50) nt of the reference polynucleotide. These are useful as diagnostic probes and primers as discussed above and in more detail below.

By a portion of a polynucleotide of “at least 20 nt in length,” for example, is intended 20 or more contiguous nucleotides from the nucleotide sequence of the reference polynucleotide (e.g., the deposited cDNA or the nucleotide sequence as shown in FIGS. 1A and 1B (SEQ ID NO:1)). Of course, a polynucleotide which hybridizes only to a poly A sequence (such as the 3′ terminal poly(A) tract of the HFLP cDNA shown in FIGS. 1A and 1B (SEQ ID NO:1)), or to a complementary stretch of T (or U) residues, would not be included in a polynucleotide of the invention used to hybridize to a portion of a nucleic acid of the invention, since such a polynucleotide would hybridize to any nucleic acid molecule containing a poly (A) stretch or the complement thereof (e.g., practically any double-stranded cDNA clone).

As indicated, nucleic acid molecules of the present invention which encode an HFLP polypeptide may include, but are not limited to those encoding the amino acid sequence of the mature polypeptide, by itself, and the coding sequence for the mature polypeptide and additional sequences, such as those encoding the about 19 or 43 amino acid leader or secretory sequence, such as a pre-, or pro- or prepro-protein sequence; the coding sequence of the mature polypeptide, with or without the aforementioned additional coding sequences.

Also encoded by nucleic acids of the invention are the above protein sequences together with additional, non-coding sequences, including for example, but not limited to introns and non-coding 5′ and 3′ sequences, such as the transcribed, non-translated sequences that play a role in transcription, mRNA processing, including splicing and polyadenylation signals, for example—ribosome binding and stability of mRNA; an additional coding sequence which codes for additional amino acids, such as those which provide additional functionalities.

Thus, the sequence encoding the polypeptide may be fused to a marker sequence, such as a sequence encoding a peptide which facilitates purification of the fused polypeptide. In certain preferred embodiments of this aspect of the invention, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described by Gentz and colleagues (Proc. Natl. Acad. Sci. USA 86:821-824 (1989)), for instance, hexa-histidine provides for convenient purification of the fusion protein. The “HA” tag is another peptide useful for purification which corresponds to an epitope derived from the influenza hemagglutinin protein, which has been described by Wilson and coworkers (Cell 37:767 (1984)). As discussed below, other such fusion proteins include the HFLP fused to Fc at the N- or C-terminus.

Variant and Mutant Polynucleotides

The present invention further relates to variants of the nucleic acid molecules of the present invention, which encode portions, analogs or derivatives of the HFLP protein. Variants may occur naturally, such as a natural allelic variant. By an “allelic variant” is intended one of several alternate forms of a gene occupying a given locus on a chromosome of an organism (Genes II, Lewin, B., ed., John Wiley & Sons, New York (1985)). Non-naturally occurring variants may be produced using art-known mutagenesis techniques.

Such variants include those produced by nucleotide substitutions, deletions or additions. The substitutions, deletions or additions may involve one or more nucleotides. The variants may be altered in coding regions, non-coding regions, or both. Alterations in the coding regions may produce conservative or non-conservative amino acid substitutions, deletions or additions. Especially preferred among these are silent substitutions, additions and deletions, which do not alter the properties and activities of the HFLP protein or portions thereof. Also especially preferred in this regard are conservative substitutions.

Most highly preferred are nucleic acid molecules encoding the mature protein having the amino acid sequence shown in SEQ ID NO:2 or the mature HFLP amino acid sequence encoded by the deposited cDNA clone.

Also most highly preferred are nucleic acid molecules encoding the predicted mature protein having the amino acid sequence shown in SEQ ID NO:4.

Most highly preferred are nucleic acid molecules encoding the frizzled domain of the protein having the amino acid sequence shown in SEQ ID NO:2 or the frizzled domain of the HFLP amino acid sequence encoded by the deposited cDNA clone.

Also most highly preferred are nucleic acid molecules encoding the predicted frizzled domain of the protein having the amino acid sequence shown in SEQ ID NO:4.

Further embodiments include an isolated nucleic acid molecule comprising a polynucleotide having a nucleotide sequence at least 90% identical, and more preferably at least 95%, 96%, 97%, 98% or 99% identical to a polynucleotide selected from the group consisting of: (a) a nucleotide sequence encoding the HFLP polypeptide having the complete amino acid sequence in SEQ ID NO:4 (i.e., positions −43 to 325 of SEQ ID NO:4); (b) a nucleotide sequence encoding the HFLP polypeptide having the complete amino acid sequence in SEQ ID NO:4 (i.e., positions −42 to 325 of SEQ ID NO:4), excluding the N-terminal methionine; (c) a nucleotide sequence encoding the predicted mature HFLP polypeptide having the amino acid sequence at positions 1 to 325 in SEQ ID NO:4; (d) a nucleotide sequence encoding the HFLP polypeptide having the complete amino acid sequence in SEQ ID NO:2 (i.e., positions −19 to 328 of SEQ ID NO:2) plus the N-terminal 12 amino acid signal peptide from the bovine Frzb molecule; (e) a nucleotide sequence encoding the HFLP polypeptide having the complete amino acid sequence in SEQ ID NO:2 (i.e., positions −19 to 328 of SEQ ID NO:2) plus the N-terminal 12 amino acid signal peptide from the bovine Frzb molecule, excluding the N-terminal methionine; (f) a nucleotide sequence encoding the HFLP polypeptide having the complete amino acid sequence in SEQ ID NO:2 (i.e., positions −19 to 328 of SEQ ID NO:2); (g) a nucleotide sequence encoding the predicted mature HFLP polypeptide having the amino acid sequence at positions 1 to 328 in SEQ ID NO:2; (h) a nucleotide sequence encoding the conserved frizzled domain of the HFLP polypeptide having the amino acid sequence in SEQ ID NO:2 (i.e., positions 6 to 126 of SEQ ID NO:2); (i) a nucleotide sequence encoding the HFLP polypeptide having the complete amino acid sequence encoded by the cDNA clone contained in ATCC Deposit No. 209140; (j) a nucleotide sequence encoding the mature HFLP polypeptide having the amino acid sequence encoded by the cDNA clone contained in ATCC Deposit No. 209140; (k) a nucleotide sequence encoding the frizzled domain of the HFLP polypeptide having the amino acid sequence encoded by the cDNA clone contained in ATCC Deposit No. 209140; and (l) a nucleotide sequence complementary to any of the nucleotide sequences in (a), (b), (c), (d), (e), (f), (g), (h), (i), (j), or (k), above.

Further embodiments of the invention include isolated nucleic acid molecules that comprise a polynucleotide having a nucleotide sequence at least 90% identical, and more preferably at least 95%, 96%, 97%, 98% or 99% identical, to any of the nucleotide sequences in (a), (b), (c), (d), (e), (f), (g), (h), (i), (O), (k), or (l), above, or a polynucleotide which hybridizes under stringent hybridization conditions to a polynucleotide in (a), (b), (c), (d), (e), (f), (g), (h), (i), (j), (k), or (l), above. This polynucleotide which hybridizes does not hybridize under stringent hybridization conditions to a polynucleotide having a nucleotide sequence consisting of only A residues or of only T residues. An additional nucleic acid embodiment of the invention relates to an isolated nucleic acid molecule comprising a polynucleotide which encodes the amino acid sequence of an epitope-bearing portion of a HFLP polypeptide having an amino acid sequence in (a), (b), (c), (d), (e), (f), (g), (h), (i), (j), or (k), above.

In specific embodiments, the polynucleotides of this invention are less than 300 kb, 200 kb, 100 kb, 50 kb, 15 kb, 10 kb or 7.5 kb in length. In a further embodiment, polynucleotides of the invention comprise at least 15 contiguous nucleotides of HFLP coding sequence, but do not comprise all or a portion of any HFLP intron. In another embodiment, the nucleic acid comprising HFLP coding sequence does not contain coding sequences of a genomic flanking gene (i.e. 5′ or 3′ to the HFLP coding sequence in the genome).

The present invention also relates to recombinant vectors, which include the isolated nucleic acid molecules of the present invention, and to host cells containing the recombinant vectors, as well as to methods of making such vectors and host cells and for using them for production of HFLP polypeptides or peptides by recombinant techniques.

By a polynucleotide having a nucleotide sequence at least, for example, 95% “identical” to a reference nucleotide sequence encoding a HFLP polypeptide is intended that the nucleotide sequence of the polynucleotide is identical to the reference sequence except that the polynucleotide sequence may include up to five point mutations per each 100 nucleotides of the reference nucleotide sequence encoding the HFLP polypeptide. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. The query sequence may be the entire sequence shown in SEQ ID NO:2 or SEQ ID NO:4, the ORF (open reading frame), or any fragement specified as described herein.

As a practical matter, whether any particular nucleic acid molecule is at least 90%, 95%, 96%, 97%, 98% or 99% identical to, for instance, the nucleotide sequences shown in SEQ ID NO:1 or SEQ ID NO:3 or to the nucleotides sequence of the deposited cDNA clone can be determined conventionally using known computer programs such as the Bestfit program (Wisconsin Sequence Analysis Package, Version 8 for Unix, Genetics Computer Group, University Research Park, 575 Science Drive, Madison, Wis. 53711). A preferred method for determing the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6:237-245). In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identiy are: Matrix=Unitary, k-tuple=4, Mismatch Penalty=1, Joining Penalty=30, Randomization Group Length=0, Cutoff Score=1, Gap Penalty=5, Gap Size Penalty 0.05, Window Size=500 or the lenght of the subject nucleotide sequence, whichever is shorter.

If the subject sequence is shorter than the query sequence because of 5′ or 3′ deletions, not because of internal deletions, a manual correction must be made to the results. This is becuase the FASTDB program does not account for 5′ and 3′ truncations of the subject sequence when calculating percent identity. For subject sequences truncated at the 5′ or 3′ ends, relative to the the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score.

For example, a 90 base subject sequence is aligned to a 100 base query sequence to determine percent identity. The deletions occur at the 5′ end of the subject sequence and therefore, the FASTDB alignment does not show a matched/alignement of the first 10 bases at 5′ end. The 10 unpaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequnce are manually corrected for. No other manual corrections are to made for the purposes of the present invention.

The present application is directed to nucleic acid molecules at least 90%, 95%, 96%, 97%, 98% or 99% identical to the nucleic acid sequence shown in FIGS. 1A and 1B (SEQ ID NO:1), FIGS. 2A and 2B (SEQ ID NO:3), or to the nucleic acid sequence of the deposited cDNA, irrespective of whether they encode a polypeptide having HFLP activity. This is because even where a particular nucleic acid molecule does not encode a polypeptide having HFLP activity, one of skill in the art would still know how to use the nucleic acid molecule, for instance, as a hybridization probe or a polymerase chain reaction (PCR) primer. Uses of the nucleic acid molecules of the present invention that do not encode a polypeptide having HFLP activity include, inter alia, (1) isolating the HFLP gene or allelic variants thereof in a cDNA library; (2) in situ hybridization (e.g., “FISH”) to metaphase chromosomal spreads to provide precise chromosomal location of the HFLP gene, as described by Verma and colleagues (Human Chromosomes: A Manual of Basic Techniques, Pergamon Press, New York (1988)); and Northern Blot analysis for detecting HFLP mRNA expression in specific tissues.

Preferred, however, are nucleic acid molecules having sequences at least 90%, 95%, 96%, 97%, 98% or 99% identical to the nucleic acid sequences shown in FIGS. 1A and 1B (SEQ ID NO:1), FIGS. 2A and 2B (SEQ ID NO:3), or to the nucleic acid sequence of the deposited cDNA which do, in fact, encode a polypeptide having HFLP protein activity. By “a polypeptide having HFLP activity” is intended polypeptides exhibiting activity similar, but not necessarily identical, to an activity of the mature HFLP protein of the invention, as measured in a particular biological assay. For example, the HFLP protein of the present invention modulates the early effects of Wnt signaling in the developing Xenopus embryos. A microinjection assay for measuring the effects of expression of ectopic factors in developing Xenopus embryos has been described by Leyns and colleagues (Cell 88:747-756 (1997)). Briefly, the assay involves microinjecting a small amount of HFLP mRNA (approximately 4 pg), either alone or in concert with a member of the Wnt family, for example Xwnt-8, into a ventral-vegetal blastomere at the 32 cell stage (Sasai, Y., et al., Cell 79:779-790 (1994)). First, however, the minimum amounts of Xwnt-8 and mouse Wnt-1 mRNA required to produce complete secondary axes (including the eye structures) in most, but not all, injected embryos is titrated. Following the titration, HFLP mRNA and either Xwnt-8 or mouse Wnt-1 mRNA are coinjected into a ventral-vegetal blastomere at the 32 cell stage and the relative success of formation of the secondary axis is determined by visual inspection of the developing embryos. Such activity is useful for determining the degree of signal transduction modulatory activity between HFLP and the normal binding, and resulting initiation of the appropriate signal transduction cascades, between one or more members of the Wnt family and the corresponding frizzled-related receptor molecules.

HFLP protein modulates secondary axis formation in a dose-dependent manner in the above-described assay. Thus, “a polypeptide having HFLP protein activity” includes polypeptides that also exhibit any of the same modulatory activities in the above-described assays in a dose-dependent manner. Although the degree of dose-dependent activity need not be identical to that of the HFLP protein, preferably, “a polypeptide having HFLP protein activity” will exhibit substantially similar dose-dependence in a given activity as compared to the HFLP protein (i.e., the candidate polypeptide will exhibit greater activity or not more than about 25-fold less and, preferably, not more than about tenfold less activity relative to the reference HFLP protein).

In addition to the above described assay for the ability of HFLP to antagonize the early effects of Xwnt-8 or mouse Wnt-1 signaling, a similar experimental system can be used to determine the ability of HFLP to antagonize the late effects of Wnt signaling in the developing Xenopus embryo. In this case, one of skill in the art will understand that Xenopus embryos injected into two dorsal blastomeres with CSKA Xwnt-8 DNA lack dorsoanterior structures, including even the eyes (Christian, J. L. and Moon, R. T. Genes Dev. 7:13-28 (1993)). When mRNA encoding an HFLP homolog, such as Frzb-1, is coinjected with Xwnt-8 mRNA into the embryo prior to placement of the embryo into the dorsal blastomere, the ventralizing effect of Xwnt-8 is antagonized and the result is the development of a moderately dorsalized embryo which contains an enlarged head structure with eyes. In the case presented herein, mRNA encoding HFLP or a mutein thereof, is substituted for that encoding Frzb-1 and the experimental procedure is then carried out. The resulting developing embryo is analyzed for the potential antagonistic effects of the HFLP protein or mutein.

Of course, due to the degeneracy of the genetic code, one of ordinary skill in the art will immediately recognize that a large number of the nucleic acid molecules having a sequence at least 90%, 95%, 96%, 97%, 98%, or 99% identical to the nucleic acid sequence of the deposited cDNA or the nucleic acid sequence shown in FIG. 1A and 1B (SEQ ID NO:1) or 2A and 2B (SEQ ID NO:3) will encode a polypeptide “having HFLP protein activity.” In fact, since degenerate variants of these nucleotide sequences all encode the same polypeptide, this will be clear to the skilled artisan even without performing the above described comparison assay. It will be further recognized in the art that, for such nucleic acid molecules that are not degenerate variants, a reasonable number will also encode a polypeptide having HFLP protein activity. This is because the skilled artisan is fully aware of amino acid substitutions that are either less likely or not likely to significantly effect protein function (e.g., replacing one aliphatic amino acid with a second aliphatic amino acid), as further described below.

Vectors and Host Cells

The present invention also relates to vectors which include the isolated DNA molecules of the present invention, host cells which are genetically engineered with the recombinant vectors, and the production of HFLP polypeptides or fragments thereof by recombinant techniques. The vector may be, for example, a phage, plasmid, viral or retroviral vector. Retroviral vectors may be replication competent or replication defective. In the latter case, viral propagation generally will occur only in complementing host cells.

The polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The DNA insert should be operatively linked to an appropriate promoter, such as the phage lambda PL promoter, the E. coli lac, trp, phoa and tac promoters, the SV40 early and late promoters and promoters of retroviral LTRs, to name a few. Other suitable promoters will be known to the skilled artisan. The expression constructs will further contain sites for transcription initiation, termination and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS, 293 and Bowes melanoma cells; and plant cells. Appropriate culture mediums and conditions for the above-described host cells are known in the art.

Vectors preferred for use in bacteria include pQE70, pQE60 and pQE-9 (QIAGEN, Inc., supra); pBS vectors, Phagescript vectors, Bluescript vectors, pNH8A, pNH16a, pNH18A, pNH46A (Stratagene); and ptrc99a, pKK223-3, pKK233-3, pDR540, pRIT5 (Pharmacia). Among preferred eukaryotic vectors are pWLNEO, pSV2CAT, pOG44, pXT1, and pSG (Stratagene); and pSVK3, pBPV, pMSG and pSVL (Pharmacia). Other suitable vectors will be readily apparent to the skilled artisan.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals (for example, Davis, et al., Basic Methods In Molecular Biology (1986)).

In addition to encompassing host cells containing the vector constructs discussed herein, the invention also encompasses primary, secondary, and immortalized host cells of vertebrate origin, particularly those of mammalian origin, that have been engineered to delete or replace endogenous genetic material (e.g. HFLP coding sequence), and/or to include genetic material (e.g. heterologous polynucleotide sequences) that is operably associated with HFLP polynucleotides of the invention, and which activates, alters, and/or amplifies endogenous HFLP polynucleotides. For example, techniques known in the art may be used to operably associate heterologous control regions (e.g. promoter and/or enhancer) and endogenous HFLP polynucleotide sequences via homologous recombination (see, e.g. U.S. Pat. No. 5,641,670, issued Jun. 24, 1997; Internation Publication No. WO 96/29411, published Sep. 26, 1996; International Publication No. WO 94/12650, published Aug. 4, 1994; Koller, et al., Proc. Natl. Acad. Sci. USA 86:8932-8935 (1989); and Zijlstra, et al., Nature 342:435-438 (1989), the disclosures of each of which are hereby incorporated by reference in their entireties).

The polypeptide may be expressed in a modified form, such as a fusion protein, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties may be added to the polypeptide to facilitate purification. Such regions may be removed prior to final preparation of the polypeptide. The addition of peptide moieties to polypeptides to engender secretion or excretion, to improve stability and to facilitate purification, among others, are familiar and routine techniques in the art. A preferred fusion protein comprises a heterologous region from immunoglobulin that is useful to stabilize and purify proteins. For example, EP-A-O 464 533 (Canadian counterpart 2045869) discloses fusion proteins comprising various portions of constant region of immunoglobulin molecules together with another human protein or part thereof. In many cases, the Fc part in a fusion protein is thoroughly advantageous for use in therapy and diagnosis and thus results, for example, in improved pharmacokinetic properties (EP-A 0232 262). On the other hand, for some uses it would be desirable to be able to delete the Fc part after the fusion protein has been expressed, detected and purified in the advantageous manner described. This is the case when Fc portion proves to be a hindrance to use in therapy and diagnosis, for example when the fusion protein is to be used as antigen for immunizations. In drug discovery, for example, human proteins, such as hIL-5, have been fused with Fc portions for the purpose of high-throughput screening assays to identify antagonists of hIL-5 (Bennett, D., et al., J. Molecular Recognition 8:52-58 (1995); Johanson, K., et al., J. Biol. Chem. 270:9459-9471 (1995)).

The HFLP protein can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification. Polypeptides of the present invention include: products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins this prokaryotic removal process is inefficient, depending on the nature of the amino acid to which the N-terminal methionine is covalently linked.

### Polypeptides and Fragments

The invention further provides an isolated HFLP polypeptide having the amino acid sequence encoded by the deposited cDNA, or the amino acid sequence in SEQ ID NO:2, or the amino acid sequence in SEQ ID NO:4, or a peptide or polypeptide comprising a portion of the above polypeptides.

Variant and Mutant Polypeptides

To improve or alter the characteristics of HFLP polypeptides, protein engineering may be employed. Recombinant DNA technology known to those skilled in the art can be used to create novel mutant proteins or muteins including single or multiple amino acid substitutions, deletions, additions or fusion proteins. Such modified polypeptides can show, e.g., enhanced activity or increased stability. In addition, they may be purified in higher yields and show better solubility than the corresponding natural polypeptide, at least under certain purification and storage conditions.

It will be further appreciated by one of skill in the art that a heterologous sequence may be included on the N-terminus of the polypeptide of the present invention to aid in secretion of the present invention from a eukaryotic cell. Specifically, the leader sequence from the bovine Frzb gene (Hoang, B., et al., J. Biol. Chem. 271:26131-26137 (1996)) maybe added to the N-terminus of HFLP to augment to secretion of HFLP from the eukaryotic cell. Nucleotides encoding the bovine signal sequence may be added to the 5′ end of a cDNA clone of the present invention so as to add, in frame, a 12 amino acid signal sequence to the N-terminus of HFLP and, consequently, to ensure secretion of HFLP from the cell.

N-Terminal and C-Terminal Deletion Mutants

For instance, for many proteins, including the extracellular domain of a membrane associated protein or the mature form(s) of a secreted protein, it is known in the art that one or more amino acids may be deleted from the N-terminus or C-terminus without substantial loss of biological function. For instance, Ron and colleagues (J. Biol. Chem., 268:2984-2988 (1993)) reported modified KGF proteins that had heparin binding activity even if 3, 8, or 27 N-terminal amino acid residues were missing. In the present case, since the protein of the invention is a member of the Frizzled polypeptide family, deletions of N-terminal amino acids up to the cysteine at position 6 of SEQ ID NO:2 may retain some biological activity such as the ability to bind Wnt family or related proteins. Polypeptides having further N-terminal deletions including the cysteine residue in SEQ ID NO:2 would not be expected to retain such biological activities because it is known that this residue in a Frizzled-related polypeptide is the first of a number of cysteine residues conserved throughout the frizzled domain which are required for forming disulfide bridges to provide structural stability needed for receptor binding and signal transduction.

However, even if deletion of one or more amino acids from the N-terminus of a protein results in modification of loss of one or more biological functions of the protein, other biological activities may still be retained. Thus, the ability of the shortened protein to induce and/or bind to antibodies which recognize the complete or mature of the protein generally will be retained when less than the majority of the residues of the complete or mature protein are removed from the N-terminus. Whether a particular polypeptide lacking N-terminal residues of a complete protein retains such immunologic activities can readily be determined by routine methods described herein and otherwise known in the art.

Accordingly, the present invention further provides polypeptides having one or more residues deleted from the amino terminus of the amino acid sequence of the HFLP shown in SEQ ID NO:2, up to the cysteine residue at position number 6, and polynucleotides encoding such polypeptides. In particular, the present invention provides polypeptides comprising the amino acid sequence of residues n¹-328 of SEQ ID NO:2, where n is an integer in the range of −19-6, and 6 is the position of the first residue from the N-terminus of the complete HFLP polypeptide (shown in SEQ ID NO:2) believed to be required for protein binding activity of the HFLP protein.

More in particular, the invention provides polynucleotides encoding polypeptides having the amino acid sequence of residues of −18 to 328, −17 to 328, −16 to 328, −15 to 328, −14 to 328, −13 to 328, −12 to 328, −11 to 328, −10 to 328, −9 to 328, −8 to 328, −7 to 328, −6 to 328, −5 to 328, −4 to 328, −3 to 328, −2 to 328, −1 to 328, 1 to 328, 2 to 328, 3 to 328, 4 to 328, 5 to 328, and 6 to 328 of SEQ ID NO:2. Polynucleotides encoding these polypeptides also are provided.

Similarly, many examples of biologically functional C-terminal deletion muteins are known. For instance, Interferon gamma shows up to ten times higher activities by deleting 8-10 amino acid residues from the carboxy terminus of the protein (Dobeli, et al., J. Biotechnology 7:199-216 (1988)). In the present case, since the protein of the invention is a member of the Frizzled polypeptide family, deletions of C-terminal amino acids up to the cysteine residue at position 118 of SEQ ID NO:2 may retain some biological activity such as the ability to bind Wnt family or related proteins. Polypeptides having further C-terminal deletions including the cysteine residue at position 118 of SEQ ID NO:2 would not be expected to retain such biological activities because it is known that this residue in a Frizzled-related polypeptide is the last of a number of cysteine residues conserved throughout the frizzled domain which are required for forming disulfide bridges to provide structural stability needed for receptor binding and signal transduction.

However, even if deletion of one or more amino acids from the C-terminus of a protein results in modification of loss of one or more biological functions of the protein, other biological activities may still be retained. Thus, the ability of the shortened protein to induce and/or bind to antibodies which recognize the complete or mature form of the protein generally will be retained when less than the majority of the residues of the complete or mature protein are removed from the C-terminus. Whether a particular polypeptide lacking C-terminal residues of a complete protein retains such immunologic activities can readily be determined by routine methods described herein and otherwise known in the art.

Accordingly, the present invention further provides polypeptides having one or more residues from the carboxy terminus of the amino acid sequence of the HFLP shown in SEQ ID NO:2, up to the cysteine residue at position 118 of SEQ ID NO:2, and polynucleotides encoding such polypeptides. In particular, the present invention provides polypeptides having the amino acid sequence of residues −19-ml¹ of the amino acid sequence in SEQ ID NO:2, where m is any integer in the range of 118 to 328, and residue 118 is the position of the first residue from the C-terminus of the complete HFLP polypeptide (shown in SEQ ID NO:2) believed to be required for protein binding of the HFLP protein.

More in particular, the invention provides polynucleotides encoding polypeptides having the amino acid sequence of residues −19 to 118, −19 to 119, −19 to 120, −19 to 121, −19 to 122, −19 to 123, −19 to 124, −19 to 125, −19 to 126, −19 to 127, −19 to 128, −19 to 129, −19 to 130, −19 to 131, −19 to 132, −19 to 133, −19 to 134, −19 to 135, −19 to 136, −19 to 137, −19 to 138, −19 to 139, −19 to 140, −19 to 141, −119 to 142, −19 to 143, −19 to 144, −19 to 145, −19 to 146, −19 to 147, −19 to 148, −19 to 149, −19 to 150, −19 to 151, −19 to 152, −19 to 153, −19 to 154, −19 to 155, −19 to 156, −19 to 157, −19 to 158, −19 to 159, −19 to 160, −19 to 161, −19 to 162, −19 to 163, −19 to 164, −19 to 165, −19 to 166, −19 to 167, −19 to 168, −19 to 169, −19 to 170, −19 to 171, −19 to 172, −19 to 173, −19 to 174, −19 to 175, −19 to 176, −19 to 177, −19 to 178, −19 to 179, −19 to 180, −19 to 181, −19 to 182, −19 to 183, −19 to 184, −19 to 185, −19 to 186, −19 to 187, −19 to 188, −19 to 189, −19 to 190, −19 to 191, −19 to 192, −19 to 193, −19 to 194, −19 to 195, −19 to 196, −19 to 197, −19 to 198, −19 to 199, −19 to 200, −19 to 201, −19 to 202, −19 to 203, −19 to 204, −19 to 205, −19 to 206, −19 to 207, −19 to 208, −19 to 209, −19 to 210, −19 to 211, −19 to 212, −19 to 213, −19 to 214, −19 to 215, −19 to 216, −19 to 217, −19 to 218, −19 to 219, −19 to 220, −19 to 221, −19 to 222, −19 to 223, −19 to 224, −19 to 225, −19 to 226, −19 to 227, −19 to 228, −19 to 229, −19 to 230, −19 to 231, −19 to 232, −19 to 233, −19 to 234, −19 to 235, −19 to 236, −19 to 237, −19 to 238, −19 to 239, −19 to 240, −19 to 241, −119 to 242, −19 to 243, −19 to 244, −19 to 245, −19 to 246, −19 to 247, −19 to 248, −19 to 249, −19 to 250, −19 to 251, −19 to 252, −19 to 253, −19 to 254, −19 to 255, −19 to 256, −19 to 257, −19 to 258, −19 to 259, −19 to 260, −19 to 261, −19 to 262, −19 to 263, −19 to 264, −19 to 265, −19 to 266, −19 to 267, −19 to 268, −19 to 269, −19 to 270, −19 to 271, −19 to 272, −19 to 273, −19 to 274, −19 to 275, −19 to 276, −19 to 277, −19 to 278, −19 to 279, −19 to 280, −19 to 281, −19 to 282, −19 to 283, −19 to 284, −19 to 285, −19 to 286, −19 to 287, −19 to 288, −19 to 289, −19 to 290, −19 to 291, −19 to 292, −19 to 293, −19 to 294, −19 to 295, −19 to 296, −19 to 297, −19 to 298, −19 to 299, −19 to 300, −19 to 301, −19 to 302, −19 to 303, −19 to 304, −19 to 305, −19 to 306, −19 to 307, −19 to 308, −19 to 309, −19 to 310, −19 to 311, −19 to 312, −19 to 313, −19 to 314, −19 to 315, −19 to 316, −19 to 317, −19 to 318, −19 to 319, −19 to 320, −19 to 321, −19 to 322, −19 to 323, −19 to 324, −19 to 325, −19 to 326, −19 to 327, and −19 to 328 of SEQ ID NO:2. Polynucleotides encoding these polypeptides also are provided.

The invention also provides polypeptides having one or more amino acids deleted from both the amino and the carboxyl termini, which may be described generally as having residues n¹-m¹ of SEQ ID NO:2, where n¹ and m¹ are integers as described above.

Also included are a nucleotide sequence encoding a polypeptide consisting of a portion of the complete HFLP amino acid sequence encoded by the cDNA clone contained in ATCC Deposit No. 209140, where this portion excludes from 1 to about 24 amino acids from the amino terminus of the complete amino acid sequence encoded by the cDNA clone contained in ATCC Deposit No. 209140, or from 1 to about 210 amino acids from the carboxy terminus, or any combination of the above amino terminal and carboxy terminal deletions, of the complete amino acid sequence encoded by the cDNA clone contained in ATCC Deposit No. 209140. Polynucleotides encoding all of the above deletion mutant polypeptide forms also are provided.

As mentioned above, even if deletion of one or more amino acids from the N-terminus of a protein results in modification of loss of one or more biological functions of the protein, other biological activities may still be retained. Thus, the ability of the shortened HFLP mutein to induce and/or bind to antibodies which recognize the complete or mature of the protein generally will be retained when less than the majority of the residues of the complete or mature protein are removed from the N-terminus. Whether a particular polypeptide lacking N-terminal residues of a complete protein retains such immunologic activities can readily be determined by routine methods described herein and otherwise known in the art. It is not unlikely that an HFLP mutein with a large number of deleted N-terminal amino acid residues may retain some biological or immungenic activities. In fact, peptides composed of as few as six HFLP amino acid residues may often evoke an immune response.

Accordingly, the present invention further provides polypeptides having one or more residues deleted from the amino terminus of the HFLP amino acid sequence shown in FIGS. 1A and 1B, up to the threonine residue at position number 342 and polynucleotides encoding such polypeptides. In particular, the present invention provides polypeptides comprising the amino acid sequence of residues n²-348 of FIGS. 1A and 1B (which is the same sequence shown in SEQ ID NO:2 except for the numbering change described below), where n² is an integer in the range of 2 to 342, and 343 is the position of the first residue from the N-terminus of the complete HFLP polypeptide believed to be required for at least immunogenic activity of the HFLP protein.

More in particular, the invention provides polynucleotides encoding polypeptides comprising, or alternatively consisting of, the amino acid sequence of residues of R-2 to V-347; V-3 to V-347; R-4 to V-347; S-5 to V-347; I-6 to V-347; L-7 to V-347; V-8 to V-347; A-9 to V-347; L-10 to V-347; C-11 to V-347; L-12 to V-347; W-13 to V-347; L-14 to V-347; H-15 to V-347; L-16 to V-347; A-17 to V-347; L-18 to V-347; G-19 to V-347; V-20 to V-347; R-21 to V-347; G-22 to V-347; A-23 to V-347; P-24 to V-347; C-25 to V-347; E-26 to V-347; A-27 to V-347; V-28 to V-347; R-29 to V-347; I-30 to V-347; P-31 to V-347; M-32 to V-347; C-33 to V-347; R-34 to V-347; H-35 to V-347; M-36 to V-347; P-37 to V-347; W-38 to V-347; N-39 to V-347; I-40 to V-347; T-41 to V-347; R-42 to V-347; M-43 to V-347; P-44 to V-347; N-45 to V-347; H-46 to V-347; L-47 to V-347; H-48 to V-347; H-49 to V-347; S-50 to V-347; T-51 to V-347; Q-52 to V-347; E-53 to V-347; N-54 to V-347; A-55 to V-347; 1-56 to V-347; L-57 to V-347; A-58 to V-347; 1-59 to V-347; E-60 to V-347; Q-61 to V-347; Y-62 to V-347; E-63 to V-347; E-64 to V-347; L-65 to V-347; V-66 to V-347; D-67 to V-347; V-68 to V-347; N-69 to V-347; C-70 to V-347; S-71 to V-347; A-72 to V-347; V-73 to V-347; L-74 to V-347; R-75 to V-347; F-76 to V-347; F-77 to V-347; L-78 to V-347; C-79 to V-347; A-80 to V-347; M-81 to V-347; Y-82 to V-347; A-83 to V-347; P-84 to V-347; I-85 to V-347; C-86 to V-347; T-87 to V-347; L-88 to V-347; E-89 to V-347; F-90 to V-347; L-91 to V-347; H-92 to V-347; D-93 to V-347; P-94 to V-347; I-95 to V-347; K-96 to V-347; P-97 to V-347; C-98 to V-347; K-99 to V-347; S-100 to V-347; V-101 to V-347; C-102 to V-347; Q-103 to V-347; R-104 to V-347; A-105 to V-347; R-106 to V-347; D-107 to V-347; D-108 to V-347; C-109 to V-347; E-110 to V-347; P-111 to V-347; L-112 to V-347; M-113 to V-347; K-114 to V-347; M-115 to V-347; Y-116 to V-347; N-117 to V-347; H-118 to V-347; S-19 to V-347; W-120 to V-347; P-121 to V-347; E-122 to V-347; S-123 to V-347; L-124 to V-347; A-125 to V-347; C-126 to V-347; D-127 to V-347; E-128 to V-347; L-129 to V-347; P-130 to V-347; V-131 to V-347; Y-132 to V-347; D-133 to V-347; R-134 to V-347; G-135 to V-347; V-136 to V-347; C-137 to V-347; I-138 to V-347; S-139 to V-347; P-140 to V-347; E-141 to V-347; A-142 to V-347; 1-143 to V-347; V-144 to V-347; T-145 to V-347; D-146 to V-347; L-147 to V-347; P-148 to V-347; E-149 to V-347; D-150 to V-347; V-151 to V-347; K-152 to V-347; W-153 to V-347; I-154 to V-347; D-155 to V-347; I-156 to V-347; T-157 to V-347; P-158 to V-347; D-159 to V-347; M-160 to V-347; M-161 to V-347; V-162 to V-347; Q-163 to V-347; E-164 to V-347; R-165 to V-347; P-166 to V-347; L-167 to V-347; D-168 to V-347; V-169 to V-347; D-170 to V-347; C-171 to V-347; K-172 to V-347; R-173 to V-347; L-174 to V-347; S-175 to V-347; P-176 to V-347; D-177 to V-347; R-178 to V-347; C-179 to V-347; K-180 to V-347; C-181 to V-347; K-182 to V-347; K-183 to V-347; V-184 to V-347; K-185 to V-347; P-186 to V-347; T-187 to V-347; L-188 to V-347; A-189 to V-347; T-190 to V-347; Y-191 to V-347; L-192 to V-347; S-193 to V-347; K-194 to V-347; N-195 to V-347; Y-196 to V-347; S-197 to V-347; Y-198 to V-347; V-199 to V-347; I-200 to V-347; H-201 to V-347; A-202-to V-347; K-203 to V-347; I-204 to V-347; K-205 to V-347; A-206 to V-347; V-207 to V-347; Q-208 to V-347; R-209 to V-347; S-210 to V-347; G-211 to V-347; C-212 to V-347; N-213 to V-347; E-214 to V-347; V-215 to V-347; T-216 to V-347; T-217 to V-347; V-218 to V-347; V-219 to V-347; D-220 to V-347; V-221 to V-347; K-222 to V-347; E-223 to V-347; I-224 to V-347; F-225 to V-347; K-226 to V-347; S-227 to V-347; S-228 to V-347; S-229 to V-347; P-230 to V-347; I-231 to V-347; P-232 to V-347; R-233 to V-347; T-234 to V-347; Q-235 to V-347; V-236 to V-347; P-237 to V-347; L-238 to V-347; I-239 to V-347; T-240 to V-347; N-241 to V-347; S-242 to V-347; S-243 to V-347; C-244 to V-347; Q-245 to V-347; C-246 to V-347; P-247 to V-347; H-248 to V-347; I-249 to V-347; L-250 to V-347; P-251 to V-347; H-252 to V-347; Q-253 to V-347; D-254 to V-347; V-255 to V-347; L-256 to V-347; I-257 to V-347; M-258 to V-347; C-259 to V-347; Y-260 to V-347; E-261 to V-347; W-262 to V-347; R-263 to V-347; S-264 to V-347; R-265 to V-347; M-266 to V-347; M-267 to V-347; L-268 to V-347; L-269 to V-347; E-270 to V-347; N-271 to V-347; C-272 to V-347; L-273 to V-347; V-274 to V-347; E-275 to V-347; K-276 to V-347; W-277 to V-347; R-278 to V-347; D-279 to V-347; Q-280 to V-347; L-281 to V-347; S-282 to V-347; K-283 to V-347; R-284 to V-347; S-285 to V-347; I-286 to V-347; Q-287 to V-347; W-288 to V-347; E-289 to V-347; E-290 to V-347; R-291 to V-347; L-292 to V-347; Q-293 to V-347; E-294 to V-347; Q-295 to V-347; R-296 to V-347; R-297 to V-347; T-298 to V-347; V-299 to V-347; Q-300 to V-347; D-301 to V-347; K-302 to V-347; K-303 to V-347; K-304 to V-347; T-305 to V-347; A-306 to V-347; G-307 to V-347; R-308 to V-347; T-309 to V-347; S-310 to V-347; R-311 to V-347; S-312 to V-347; N-313 to V-347; P-314 to V-347; P-315 to V-347; K-316 to V-347; P-317 to V-347; K-318 to V-347; G-319 to V-347; K-320 to V-347; T-321 to V-347; P-322 to V-347; A-323 to V-347; P-324 to V-347; K-325 to V-347; P-326 to V-347; A-327 to V-347; S-328 to V-347; P-329 to V-347; K-330 to V-347; K-331 to V-347; N-332 to V-347; 1-333 to V-347; K-334 to V-347; T-335 to V-347; R-336 to V-347; S-337 to V-347; A-338 to V-347; Q-339 to V-347; K-340 to V-347; R-341 to V-347; T-342 to V-347 of the HFLP sequence shown in FIGS. 1A and 1B (which is identical to the sequence shown as SEQ ID NO:2, with the exception that the amino acid residues in FIGS. 1A and 1B are numbered consecutively from 1 through 347 from the N-terminus to the C-terminus, while the amino acid residues in SEQ ID NO:2 are numbered consecutively from −19 through 328 to reflect the position of the predicted signal peptide). Polynucleotides encoding these polypeptides are also encompassed by the invention.

Also as mentioned above, even if deletion of one or more amino acids from the C-terminus of a protein results in modification of loss of one or more biological functions of the protein, other biological activities may still be retained. Thus, the ability of the shortened HFLP mutein to induce and/or bind to antibodies which recognize the complete or mature of the protein generally will be retained when less than the majority of the residues of the complete or mature protein are removed from the C-terminus. Whether a particular polypeptide lacking C-terminal residues of a complete protein retains such immunologic activities can readily be determined by routine methods described herein and otherwise known in the art. It is not unlikely that an HFLP mutein with a large number of deleted C-terminal amino acid residues may retain some biological or immungenic activities. In fact, peptides composed of as few as six HFLP amino acid residues may often evoke an immune response.

Accordingly, the present invention further provides polypeptides having one or more residues deleted from the carboxy terminus of the amino acid sequence of the HFLP shown in FIGS. 1A and 1B, up to the isoleucine residue at position number 6, and polynucleotides encoding such polypeptides. In particular, the present invention provides polypeptides comprising the amino acid sequence of residues 1-m² of FIGS. 1A and 1B (which is the same sequence shown in SEQ ID NO:2 except for the numbering change described below), where m² is an integer in the range of 6 to 347, and 6 is the position of the first residue from the C-terminus of the complete HFLP polypeptide believed to be required for at least immunogenic activity of the HFLP protein.

More in particular, the invention provides polynucleotides encoding polypeptides comprising, or alternatively consisting of, the amino acid sequence of residues P-1 to R-346; P-1 to K-345; P-1 to P-344; P-1 to N-343; P-1 to T-342; P-1 to R-341; P-1 to K-340; P-1 to Q-339; P-1 to A-338; P-1 to S-337; P-1 to R-336; P-1 to T-335; P-1 to K-334; P-1 to I-333; P-1 to N-332; P-1 to K-331; P-1 to K-330; P-1 to P-329; P-1 to S-328; P-1 to A-327; P-1 to P-326; P-1 to K-325; P-1 to P-324; P-1 to A-323; P-1 to P-322; P-1 to T-321; P-1 to K-320; P-1 to G-319; P-1 to K-318; P-1 to P-317; P-1 to K-316; P-1 to P-315; P-1 to P-314; P-1 to N-313; P-1 to S-312; P-1 to R-311; P-1 to S-310; P-1 to T-309; P-1 to R-308; P-1 to G-307; P-1 to A-306; P-1 to T-305; P-1 to K-304; P-1 to K-303; P-1 to K-302; P-1 to D-301; P-1 to Q-300; P-1 to V-299; P-1 to T-298; P-1 to R-297; P-1 to R-296; P-1 to Q-295; P-1 to E-294; P-1 to Q-293; P-1 to L-292; P-1 to R-291; P-1 to E-290; P-1 to E-289; P-1 to W-288; P-1 to Q-287; P-1 to I-286; P-1 to S-285; P-1 to R-284; P-1 to K-283; P-1 to S-282; P-1 to L-281; P-1 to Q-280; P-1 to D-279; P-1 to R-278; P-1 to W-277; P-1 to K-276; P-1 to E-275; P-1 to V-274; P-1 to L-273; P-1 to C-272; P-1 to N-271; P-1 to E-270; P-1 to L-269; P-1 to L-268; P-1 to M-267; P-1 to M-266; P-1 to R-265; P-1 to S-264; P-1 to R-263; P-1 to W-262; P-1 to E-261; P-1 to Y-260; P-1 to C-259; P-1 to M-258; P-1 to I-257; P-1 to L-256; P-1 to V-255; P-1 to D-254; P-1 to Q-253; P-1 to H-252; P-1 to P-251; P-1 to L-250; P-1 to I-249; P-1 to H-248; P-1 to P-247; P-1 to C-246; P-1 to Q-245; P-1 to C-244; P-1 to S-243; P-1 to S-242; P-1 to N-241; P-1 to T-240; P-1 to I-239; P-1 to L-238; P-1 to P-237; P-1 to V-236; P-1 to Q-235; P-1 to T-234; P-1 to R-233; P-1 to P-232; P-1 to I-231; P-1 to P-230; P-1 to S-229; P-1 to S-228; P-1 to S-227; P-1 to K-226; P-1 to F-225; P-1 to I-224; P-1 to E-223; P-1 to K-222; P-1 to V-221; P-1 to D-220; P-1 to V-219; P-1 to V-218; P-1 to T-217; P-1 to T-216; P-1 to V-215; P-1 to E-214; P-1 to N-213; P-1 to C-212; P-1 to G-211; P-1 to S-210; P-1 to R-209; P-1 to Q-208; P-1 to V-207; P-1 to A-206; P-1 to K-205; P-1 to 1-204; P-1 to K-203; P-1 to A-202; P-1 to H-201; P-1 to I-200; P-1 to V-199; P-1 to Y-198; P-1 to S-197; P-1 to Y-196; P-1 to N-195; P-1 to K-194; P-1 to S-193; P-1 to L-192; P-1 to Y-191; P-1 to T-190; P-1 to A-189; P-1 to L-188; P-1 to T-187; P-1 to P-186; P-1 to K-185; P-1 to V-184; P-1 to K-183; P-1 to K-182; P-1 to C-181; P-1 to K-180; P-1 to C-179; P-1 to R-178; P-1 to D-177; P-1 to P-176; P-1 to S-175; P-1 to L-174; P-1 to R-173; P-1 to K-172; P-1 to C-171; P-1 to D-170; P-1 to V-169; P-1 to D-168; P-1 to L-167; P-1 to P-166; P-1 to R-165; P-1 to E-164; P-1 to Q-163; P-1 to V-162; P-1 to M-161; P-1 to M-160; P-1 to D-159; P-1 to P-158; P-1 to T-157; P-1 to I-156; P-1 to D-155; P-1 to I-154; P-1 to W-153; P-1 to K-152; P-1 to V-151; P-1 to D-150; P-1 to E-149; P-1 to P-148; P-1 to L-147; P-1 to D-146; P-1 to T-145; P-1 to V-144; P-1 to I-143; P-1 to A-142; P-1 to E-141; P-1 to P-140; P-1 to S-139; P-1 to 1-138; P-1 to C-137; P-1 to V-136; P-1 to G-135; P-1 to R-134; P-1 to D-133; P-1 to Y-132; P-1 to V-131; P-1 to P-130; P-1 to L-129; P-1 to E-128; P-1 to D-127; P-1 to C-126; P-1 to A-125; P-1 to L-124; P-1 to S-123; P-1 to E-122; P-1 to P-121; P-1 to W-120; P-1 to S-119; P-1 to H-118; P-1 to N-117; P-1 to Y-116; P-1 to M-115; P-1 to K-114; P-1 to M-113; P-1 to L-112; P-1 to P-111; P-1 to E-110; P-1 to C-109; P-1 to D-108; P-1 to D-107; P-1 to R-106; P-1 to A-105; P-1 to R-104; P-1 to Q-103; P-1 to C-102; P-1 to V-101; P-1 to S-100; P-1 to K-99; P-1 to C-98; P-1 to P-97; P-1 to K-96; P-1 to 1-95; P-1 to P-94; P-1 to D-93; P-1 to H-92; P-1 to L-91; P-1 to F-90; P-1 to E-89; P-1 to L-88; P-1 to T-87; P-1 to C-86; P-1 to I-85; P-1 to P-84; P-1 to A-83; P-1 to Y-82; P-1 to M-81; P-1 to A-80; P-1 to C-79; P-1 to L-78; P-1 to F-77; P-1 to F-76; P-1 to R-75; P-1 to L-74; P-1 to V-73; P-1 to A-72; P-1 to S-71; P-1 to C-70; P-1 to N-69; P-1 to V-68; P-1 to D-67; P-1 to V-66; P-1 to L-65; P-1 to E-64; P-1 to E-63; P-1 to Y-62; P-1 to Q-61; P-1 to E-60; P-1 to I-59; P-1 to A-58; P-1 to L-57; P-1 to I-56; P-1 to A-55; P-1 to N-54; P-1 to E-53; P-1 to Q-52; P-1 to T-51; P-1 to S-50; P-1 to H-49; P-1 to H-48; P-1 to L-47; P-1 to H-46; P-1 to N-45; P-1 to P-44; P-1 to M-43; P-1 to R-42; P-1 to T-41; P-1 to 1-40; P-1 to N-39; P-1 to W-38; P-1 to P-37; P-1 to M-36; P-1 to H-35; P-1 to R-34; P-1 to C-33; P-1 to M-32; P-1 to P-31; P-1 to I-30; P-1 to R-29; P-1 to V-28; P-1 to A-27; P-1 to E-26; P-1 to C-25; P-1 to P-24; P-1 to A-23; P-1 to G-22; P-1 to R-21; P-1 to V-20; P-1 to G-19; P-1 to L-18; P-1 to A-17; P-1 to L-16; P-1 to H-15; P-1 to L-14; P-1 to W-13; P-1 to L-12; P-1 to C-11; P-1 to L-10; P-1 to A-9; P-1 to V-8; P-1 to L-7; P-1 to 1-6 of the sequence of the HFLP sequence shown in Figures 1A and 1B (which is identical to the sequence shown as SEQ ID NO:2, with the exception that the amino acid residues in FIGS. 1A and 1B are numbered consecutively from 1 through 347 from the N-terminus to the C-terminus, while the amino acid residues in SEQ ID NO:2 are numbered consecutively from −19 through 328 to reflect the position of the predicted signal peptide). Polynucleotides encoding these polypeptides also are provided.

The invention also provides polypeptides having one or more amino acids deleted from both the amino and the carboxyl termini of an HFLP polypeptide, which may be described generally as having residues n²-m² of FIGS. 1A and 1B (i.e., the sequence of SEQ ID NO:2 numbered according to the scheme of FIGS. 1A and 1B), where n² and m² are integers as described above.

Also as mentioned above, even if deletion of one or more amino acids from the N-terminus of a protein results in modification of loss of one or more biological functions of the protein, other biological activities may still be retained. Thus, the ability of the shortened HFLP mutein to induce and/or bind to antibodies which recognize the complete or mature of the protein generally will be retained when less than the majority of the residues of the complete or mature protein are removed from the N-terminus. Whether a particular polypeptide lacking N-terminal residues of a complete protein retains such immunologic activities can readily be determined by routine methods described herein and otherwise known in the art. It is not unlikely that an HFLP mutein with a large number of deleted N-terminal amino acid residues may retain some biological or immungenic activities. In fact, peptides composed of as few as six HFLP amino acid residues may often evoke an immune response.

Accordingly, the present invention further provides polypeptides having one or more residues deleted from the amino terminus of the HFLP amino acid sequence shown in SEQ ID NO:4, up to the threonine residue at position number 363 of the sequence shown in FIGS. 2A and 2B and polynucleotides encoding such polypeptides. In particular, the present invention provides polypeptides comprising the amino acid sequence of residues n³-368 of FIGS. 2A and 2B (which is the same sequence shown in SEQ ID, NO:4 except for the numbering change described below), where n³ is an integer in the range of 2 to 363, and 364 is the position of the first residue from the N-terminus of the complete HFLP polypeptide believed to be required for at least immunogenic activity of the HFLP protein.

More in particular, the invention provides polynucleotides encoding polypeptides comprising, or alternatively consisting of, the amino acid sequence of residues of R-2 to V-368; V-3 to V-368; A-4 to V-368; G-5 to V-368; R-6 to V-368; E-7 to V-368; G-8 to V-368; R-9 to V-368; F-10 to V-368; L-11 to V-368; S-12 to V-368; A-13 to V-368; G-14 to V-368; V-15 to V-368; A-16 to V-368; A-17 to V-368; R-18 to V-368; E-19 to V-368; G-20 to V-368; S-21 to V-368; A-22 to V-368; M-23 to V-368; F-24 to V-368; L-25 to V-368; S-26 to V-368; I-27 to V-368; L-28 to V-368; V-29 to V-368; A-30 to V-368; L-31 to V-368; C-32 to V-368; L-33 to V-368; W-34 to V-368; L-35 to V-368; H-36 to V-368; L-37 to V-368; A-38 to V-368; L-39 to V-368; G-40 to V-368; V-41 to V-368; R-42 to V-368; G-43 to V-368; A-44 to V-368; P-45 to V-368; C-46 to V-368; E-47 to V-368; A-48 to V-368; V-49 to V-368; R-50 to V-368; I-51 to V-368; P-52 to V-368; M-53 to V-368; C-54 to V-368; R-55 to V-368; H-56 to V-368; M-57 to V-368; P-58 to V-368; W-59 to V-368; N-60 to V-368; I-61 to V-368; T-62 to V-368; R-63 to V-368; M-64 to V-368; P-65 to V-368; N-66 to V-368; H-67 to V-368; L-68 to V-368; H-69 to V-368; H-70 to V-368; S-71 to V-368; T-72 to V-368; Q-73 to V-368; E-74 to V-368; N-75 to V-368; A-76 to V-368; I-77 to V-368; L-78 to V-368; A-79 to V-368; I-80 to V-368; E-81 to V-368; Q-82 to V-368; Y-83 to V-368; E-84 to V-368; E-85 to V-368; L-86 to V-368; V-87 to V-368; D-88 to V-368; V-89 to V-368; N-90 to V-368; C-91 to V-368; S-92 to V-368; A-93 to V-368; V-94 to V-368; L-95 to V-368; R-96 to V-368; F-97 to V-368; F-98 to V-368; L-99 to V-368; C-100 to V-368; A-101 to V-368; M-102 to V-368; Y-103 to V-368; A-104 to V-368; P-105 to V-368; I-106 to V-368; C-107 to V-368; T-108 to V-368; L-109 to V-368; E-110 to V-368; F-111 to V-368; L-112 to V-368; H-113 to V-368; D-114 to V-368; P-115 to V-368; I-116 to V-368; K-117 to V-368; P-118 to V-368; C-119 to V-368; K-120 to V-368; S-121 to V-368; V-122 to V-368; C-123 to V-368; Q-124 to V-368; R-125 to V-368; A-126 to V-368; R-127 to V-368; D-128 to V-368; D-129 to V-368; C-130 to V-368; E-131 to V-368; P-132 to V-368; L-133 to V-368; M-134 to V-368; K-135 to V-368; M-136 to V-368; Y-137 to V-368; N-138 to V-368; H-139 to V-368; S-140 to V-368; W-141 to V-368; P-142 to V-368; E-143 to V-368; S-144 to V-368; L-145 to V-368; A-146 to V-368; C-147 to V-368; D-148 to V-368; E-149 to V-368; L-150 to V-368; P-151 to V-368; V-152 to V-368; Y-153 to V-368; D-154 to V-368; R-155 to V-368; G-156 to V-368; V-157 to V-368; C-158 to V-368; I-159 to V-368; S-160 to V-368; P-161 to V-368; E-162 to V-368; A-163 to V-368; I-164 to V-368; V-165 to V-368; T-166 to V-368; D-167 to V-368; L-168 to V-368; P-169 to V-368; E-170 to V-368; D-171 to V-368; V-172 to V-368; K-173 to V-368; W-174 to V-368; I-175 to V-368; D-176 to V-368; I-177 to V-368; T-178 to V-368; P-179 to V-368; D-180 to V-368; M-181 to V-368; M-182 to V-368; V-183 to V-368; Q-184 to V-368; E-185 to V-368; R-186 to V-368; P-187 to V-368; L-188 to V-368; D-189 to V-368; V-190 to V-368; D-191 to V-368; C-192 to V-368; K-193 to V-368; R-194 to V-368; L-195 to V-368; S-196 to V-368; P-197 to V-368; D-198 to V-368; R-199 to V-368; C-200 to V-368; K-201 to V-368; C-202 to V-368; K-203 to V-368; K-204 to V-368; V-205 to V-368; K-206 to V-368; P-207 to V-368; T-208 to V-368; L-209 to V-368; A-210 to V-368; T-211 to V-368; Y-212 to V-368; L-213 to V-368; S-214 to V-368; K-215 to V-368; N-216 to V-368; Y-217 to V-368; S-218 to V-368; Y-219 to V-368; V-220 to V-368; I-221 to V-368; H-222 to V-368; A-223 to V-368; K-224 to V-368; 1-225 to V-368; K-226 to V-368; A-227 to V-368; V-228 to V-368; Q-229 to V-368; R-230 to V-368; S-231 to V-368; G-232 to V-368; C-233 to V-368; N-234 to V-368; E-235 to V-368; V-236 to V-368; T-237 to V-368; T-238 to V-368; V-239 to V-368; V-240 to V-368; D-241 to V-368; V-242 to V-368; K-243 to V-368; E-244 to V-368; I-245 to V-368; F-246 to V-368; K-247 to V-368; S-248 to V-368; S-249 to V-368; S-250 to V-368; P-251 to V-368; I-252 to V-368; P-253 to V-368; R-254 to V-368; T-255 to V-368; Q-256 to V-368; V-257 to V-368; P-258 to V-368; L-259 to V-368; I-260 to V-368; T-261 to V-368; N-262 to V-368; S-263 to V-368; S-264 to V-368; C-265 to V-368; Q-266 to V-368; C-267 to V-368; P-268 to V-368; H-269 to V-368; I-270 to V-368; L-271 to V-368; P-272 to V-368; H-273 to V-368; Q-274 to V-368; D-275 to V-368; V-276 to V-368; L-277 to V-368; I-278 to V-368; M-279 to V-368; C-280 to V-368; Y-281 to V-368; E-282 to V-368; W-283 to V-368; R-284 to V-368; S-285 to V-368; R-286 to V-368; M-287 to V-368; M-288 to V-368; L-289 to V-368; L-290 to V-368; E-291 to V-368; N-292 to V-368; C-293 to V-368; L-294 to V-368; V-295 to V-368; E-296 to V-368; K-297 to V-368; W-298 to V-368; R-299 to V-368; D-300 to V-368; Q-301 to V-368; L-302 to V-368; S-303 to V-368; K-304 to V-368; R-305 to V-368; S-306 to V-368; I-307 to V-368; Q-308 to V-368; W-309 to V-368; E-310 to V-368; E-311 to V-368; R-312 to V-368; L-313 to V-368; Q-314 to V-368; E-315 to V-368; Q-316 to V-368; R-317 to V-368; R-318 to V-368; T-319 to V-368; V-320 to V-368; Q-321 to V-368; D-322 to V-368; K-323 to V-368; K-324 to V-368; K-325 to V-368; T-326 to V-368; A-327 to V-368; G-328 to V-368; R-329 to V-368; T-330 to V-368; S-331 to V-368; R-332 to V-368; S-333 to V-368; N-334 to V-368; P-335 to V-368; P-336 to V-368; K-337 to V-368; P-338 to V-368; K-339 to V-368; G-340 to V-368; K-341 to V-368; T-342 to V-368; P-343 to V-368; A-344 to V-368; P-345 to V-368; K-346 to V-368; P-347 to V-368; A-348 to V-368; S-349 to V-368; P-350 to V-368; K-351 to V-368; K-352 to V-368; N-353 to V-368; I-354 to V-368; K-355 to V-368; T-356 to V-368; R-357 to V-368; S-358 to V-368; A-359 to V-368; Q-360 to V-368; K-361 to V-368; R-362 to V-368; and T-363 to V-368 of the HFLP sequence shown in FIGS. 2A and 2B (which is identical to the sequence shown as SEQ ID NO:4, with the exception that the amino acid residues in FIGS. 2A and 2B are numbered consecutively from 1 through 368 from the N-terminus to the C-terminus, while the amino acid residues in SEQ ID NO:4 are numbered consecutively from −43 through 325 to reflect the position of the predicted signal peptide). Polynucleotides encoding these polypeptides are also encompassed by the invention.

Also as mentioned above, even if deletion of one or more amino acids from the C-terminus of a protein results in modification of loss of one or more biological functions of the protein, other biological activities may still be retained. Thus, the ability of the shortened HFLP mutein to induce and/or bind to antibodies which recognize the complete or mature of the protein generally will be retained when less than the majority of the residues of the complete or mature protein are removed from the C-terminus. Whether a particular polypeptide lacking C-terminal residues of a complete protein retains such immunologic activities can readily be determined by routine methods described herein and otherwise known in the art. It is not unlikely that an HFLP mutein with a large number of deleted C-terminal amino acid residues may retain some biological or immungenic activities. In fact, peptides composed of as few as six HFLP amino acid residues may often evoke an immune response.

Accordingly, the present invention further provides polypeptides having one or more residues deleted from the carboxy terminus of the amino acid sequence of the HFLP shown in SEQ ID NO:4, up to the arginine residue at position number 366 of the sequence shown in FIGS. 2A and 2B and polynucleotides encoding such polypeptides. In particular, the present invention provides polypeptides comprising the amino acid sequence of residues 1-m³ of FIGS. 2A and 2B (which is the same sequence shown in SEQ ID NO:4 except for the numbering change described below), where m³ is an integer in the range of 6 to 367 and 6 is the position of the first residue from the C-terminus of the complete HFLP polypeptide believed to be required for at least immunogenic activity of the HFLP protein.

More in particular, the invention provides polynucleotides encoding polypeptides comprising, or alternatively consisting of, the amino acid sequence of residues M-1 to R-367; M-1 to K-366; M-1 to P-365; M-1 to N-364; M-1 to T-363; M-1 to R-362; M-1 to K-361; M-1 to Q-360; M-1 to A-359; M-1 to S-358; M-1 to R-357; M-1 to T-356; M-1 to K-355; M-1 to I-354; M-1 to N-353; M-1 to K-352; M-1 to K-351; M-1 to P-350; M-1 to S-349; M-1 to A-348; M-1 to P-347; M-1 to K-346; M-1 to P-345; M-1 to A-344; M-1 to P-343; M-1 to T-342; M-1 to K-341; M-1 to G-340; M-1 to K-339; M-1 to P-338; M-1 to K-337; M-1 to P-336; M-1 to P-335; M-1 to N-334; M-1 to S-333; M-1 to R-332; M-1 to S-331; M-1 to T-330; M-1 to R-329; M-1 to G-328; M-1 to A-327; M-1 to T-326; M-1 to K-325; M-1 to K-324; M-1 to K-323; M-1 to D-322; M-1 to Q-321; M-1 to V-320; M-1 to T-319; M-1 to R-318; M-1 to R-317; M-1 to Q-316; M-1 to E-315; M-1 to Q-314; M-1 to L-313; M-1 to R-312; M-1 to E-311; M-1 to E-310; M-1 to W-309; M-1 to Q-308; M-1 to I-307; M-1 to S-306; M-1 to R-305; M-1 to K-304; M-1 to S-303; M-1 to L-302; M-1 to Q-301; M-1 to D-300; M-1 to R-299; M-1 to W-298; M-1 to K-297; M-1 to E-296; M-1 to V-295; M-1 to L-294; M-1 to C-293; M-1 to N-292; M-1 to E-291; M-1 to L-290; M-1 to L-289; M-1 to M-288; M-1 to M-287; M-1 to R-286; M-1 to S-285; M-1 to R-284; M-1 to W-283; M-1 to E-282; M-1 to Y-281; M-1 to C-280; M-1 to M-279; M-1 to 1-278; M-1 to L-277; M-1 to V-276; M-1 to D-275; M-1 to Q-274; M-1 to H-273; M-1 to P-272; M-1 to L-271; M-1 to 1-270; M-1 to H-269; M-1 to P-268; M-1 to C-267; M-1 to Q-266; M-1 to C-265; M-1 to S-264; M-1 to S-263; M-1 to N-262; M-1 to T-261; M-1 to 1-260; M-1 to L-259; M-1 to P-258; M-1 to V-257; M-1 to Q-256; M-1 to T-255; M-1 to R-254; M-1 to P-253; M-1 to 1-252; M-1 to P-251; M-1 to S-250; M-1 to S-249; M-1 to S-248; M-1 to K-247; M-1 to F-246; M-1 to 1-245; M-1 to E-244; M-1 to K-243; M-1 to V-242; M-1 to D-241; M-1 to V-240; M-1 to V-239; M-1 to T-238; M-1 to T-237; M-1 to V-236; M-1 to E-235; M-1 to N-234; M-1 to C-233; M-1 to G-232; M-1 to S-231; M-1 to R-230; M-1 to Q-229; M-1 to V-228; M-1 to A-227; M-1 to K-226; M-1 to 1-225; M-1 to K-224; M-1 to A-223; M-1 to H-222; M-1 to I-221; M-1 to V-220; M-1 to Y-219; M-1 to S-218; M-1 to Y-217; M-1 to N-216; M-1 to K-215; M-1 to S-214; M-1 to L-213; M-1 to Y-212; M-1 to T-211; M-1 to A-210; M-1 to L-209; M-1 to T-208; M-1 to P-207; M-1 to K-206; M-1 to V-205; M-1 to K-204; M-1 to K-203; M-1 to C-202; M-1 to K-201; M-1 to C-200; M-1 to R-199; M-1 to D-198; M-1 to P-197; M-1 to S-196; M-1 to L-195; M-1 to R-194; M-1 to K-193; M-1 to C-192; M-1 to D-191; M-1 to V-190; M-1 to D-189; M-1 to L-188; M-1 to P-187; M-1 to R-186; M-1 to E-185; M-1 to Q-184; M-1 to V-183; M-1 to M-182; M-1 to M-181; M-1 to D-180; M-1 to P-179; M-1 to T-178; M-1 to I-177; M-1 to D-176; M-1 to 1-175; M-1 to W-174; M-1 to K-173; M-1 to V-172; M-1 to D-171; M-1 to E-170; M-1 to P-169; M-1 to L-168; M-1 to D-167; M-1 to T-166; M-1 to V-165; M-1 to 1-164; M-1 to A-163; M-1 to E-162; M-1 to P-161; M-1 to S-160; M-1 to I-159; M-1 to C-158; M-1 to V-157; M-1 to G-156; M-1 to R-155; M-1 to D-154; M-1 to Y-153; M-1 to V-152; M-1 to P-151; M-1 to L-150; M-1 to E-149; M-1 to D-148; M-1 to C-147; M-1 to A-146; M-1 to L-145; M-1 to S-144; M-1 to E-143; M-1 to P-142; M-1 to W-141; M-1 to S-140; M-1 to H-139; M-1 to N-138; M-1 to Y-137; M-1 to M-136; M-1 to K-135; M-1 to M-134; M-1 to L-133; M-1 to P-132; M-1 to E-131; M-1 to C-130; M-1 to D-129; M-1 to D-128; M-1 to R-127; M-1 to A-126; M-1 to R-125; M-1 to Q-124; M-1 to C-123; M-1 to V-122; M-1 to S-121; M-1 to K-120; M-1 to C-119; M-1 to P-118; M-1 to K-117; M-1 to I-116; M-1 to P-115; M-1 to D-114; M-1 to H-113; M-1 to L-112; M-1 to F-111; M-1 to E-110; M-1 to L-109; M-1 to T-108; M-1 to C-107; M-1 to I-106; M-1 to P-105; M-1 to A-104; M-1 to Y-103; M-1 to M-102; M-1 to A-101; M-1 to C-100; M-1 to L-99; M-1 to F-98; M-1 to F-97; M-1 to R-96; M-1 to L-95; M-1 to V-94; M-1 to A-93; M-1 to S-92; M-1 to C-91; M-1 to N-90; M-1 to V-89; M-1 to D-88; M-1 to V-87; M-1 to L-86; M-1 to E-85; M-1 to E-84; M-1 to Y-83; M-1 to Q-82; M-1 to E-81; M-1 to I-80; M-1 to A-79; M-1 to L-78; M-1 to I-77; M-1 to A-76; M-1 to N-75; M-1 to E-74; M-1 to Q-73; M-1 to T-72; M-1 to S-71; M-1 to H-70; M-1 to H-69; M-1 to L-68; M-1 to H-67; M-1 to N-66; M-1 to P-65; M-1 to M-64; M-1 to R-63; M-1 to T-62; M-1 to I-61; M-1 to N-60; M-1 to W-59; M-1 to P-58; M-1 to M-57; M-1 to H-56; M-1 to R-55; M-1 to C-54; M-1 to M-53; M-1 to P-52; M-1 to I-51; M-1 to R-50; M-1 to V-49; M-1 to A-48; M-1 to E-47; M-1 to C-46; M-1 to P-45; M-1 to A-44; M-1 to G-43; M-1 to R-42; M-1 to V-41; M-1 to G-40; M-1 to L-39; M-1 to A-38; M-1 to L-37; M-1 to H-36; M-1 to L-35; M-1 to W-34; M-1 to L-33; M-1 to C-32; M-1 to L-31; M-1 to A-30; M-1 to V-29; M-1 to L-28; M-1 to I-27; M-1 to S-26; M-1 to L-25; M-1 to F-24; M-1 to M-23; M-1 to A-22; M-1 to S-21; M-1 to G-20; M-1 to E-19; M-1 to R-18; M-1 to A-17; M-1 to A-16; M-1 to V-15; M-1 to G-14; M-1 to A-13; M-1 to S-12; M-1 to L-11; M-1 to F-10; M-1 to R-9; M-1 to G-8; M-1 to E-7; M-1 to R-6 of the sequence of the HFLP sequence shown in FIGS. 2A and 2B (which is identical to the sequence shown as SEQ ID NO:4, with the exception that the amino acid residues in FIGS. 2A and 2B are numbered consecutively from 1 through 368 from the N-terminus to the C-terminus, while the amino acid residues in SEQ ID NO:4 are numbered consecutively from −43 through 325 to reflect the position of the predicted signal peptide). Polynucleotides encoding these polypeptides also are provided.

The invention also provides polypeptides having one or more amino acids deleted from both the amino and the carboxyl termini of an HFLP polypeptide, which may be described generally as having residues n³-m³ of FIGS. 2A and 2B (i.e., the sequence of SEQ ID NO:4 numbered according to the scheme of FIGS. 2A and 2B), where n³ and m³ are integers as described above.

Other Mutants

In addition to terminal deletion forms of the protein discussed above, it also will be recognized by one of ordinary skill in the art that some amino acid sequences of the HFLP polypeptide can be varied without significant effect of the structure or function of the protein. If such differences in sequence are contemplated, it should be remembered that there will be critical areas on the protein which determine activity.

Thus, the invention further includes variations of the HFLP polypeptide which show substantial HFLP polypeptide activity or which include regions of HFLP protein such as the protein portions discussed below. Such mutants include deletions, insertions, inversions, repeats, and type substitutions selected according to general rules known in the art so as have little effect on activity. For example, guidance concerning how to make phenotypically silent amino acid substitutions is provided wherein the authors indicate that there are two main approaches for studying the tolerance of an amino acid sequence to change (Bowie, J. U., et al., Science 247:1306-1310 (1990)). The first method relies on the process of evolution, in which mutations are either accepted or rejected by natural selection. The second approach uses genetic engineering to introduce amino acid changes at specific positions of a cloned gene and selections or screens to identify sequences that maintain functionality.

As the authors state, these studies have revealed that proteins are surprisingly tolerant of amino acid substitutions. The authors further indicate which amino acid changes are likely to be permissive at a certain position of the protein. For example, most buried amino acid residues require nonpolar side chains, whereas few features of surface side chains are generally conserved. Other such phenotypically silent substitutions are described by Bowie and coworkers (supra) and the references cited therein. Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr.

Thus, the fragment, derivative or analog of the polypeptide of SEQ ID NO:2, or that encoded by the deposited cDNA, may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the above form of the polypeptide, such as an IgG Fc fusion region peptide or leader or secretory sequence or a sequence which is employed for purification of the above form of the polypeptide or a proprotein sequence. Such fragments, derivatives and analogs are deemed to be within the scope of those skilled in the art from the teachings herein.

Thus, the HFLP of the present invention may include one or more amino acid substitutions, deletions or additions, either from natural mutations or human manipulation. As indicated, changes are preferably of a minor nature, such as conservative amino acid substitutions that do not significantly affect the folding or activity of the protein (see Table 1).

TABLE 1
Conservative Amino Acid Substitutions.

| | |
|---|---|
| Aromatic | Phenylalanine |
| | Tryptophan |
| | Tyrosine |
| Hydrophobic | Leucine |
| | Isoleucine |
| | Valine |
| Polar | Glutamine |
| | Asparagine |
| Basic | Arginine |
| | Lysine |
| | Histidine |
| Acidic | Aspartic Acid |
| | Glutamic Acid |
| Small | Alanine |
| | Serine |
| | Threonine |
| | Methionine |
| | Glycine |

at every residue in the molecule. The resulting mutant molecules are then tested Embodiments of the invention are directed to polypeptides which comprise the amino acid sequence of an HFLP polypeptide described herein, but having an amino acid sequence which contains at least one conservative amino acid substitution, but not more than 50 conservative amino acid substitutions, even more preferably, not more than 40 conservative amino acid substitutions, still more preferably, not more than 30 conservative amino acid substitutions, and still even more preferably, not more than 20 conservative amino acid substitutions, when compared with the HFLP polynucleotide sequence described herein. Of course, in order of ever-increasing preference, it is highly preferable for a peptide or polypeptide to have an amino acid sequence which comprises the amino acid sequence of an HFLP polypeptide, which contains at least one, but not more than 10, 9, 8, 7, 6, 5, 4, 3, 2 or 1 conservative amino acid substitutions.

In further specific embodiments, the number of substitutions, additions or deletions in the amino acid sequence of FIGS. 1A and 1B (SEQ ID NO:2) and FIGS. 2A and 2B (SEQ ID NO:4), a polypeptide sequence encoded by the deposited clone, and/or any of the polypeptide fragments described herein is 100, 75, 70, 60, 50, 40, 35, 30, 25, 20, 15, 10, 9, 8, 7, 6, 5, 4, 3, 2, 1 or 150-50, 100-50, 50-20, 30-20, 20-15, 20-10, 15-10, 10-1,5-10, 1-5,1-3 or 1-2.

Amino acids in the HFLP protein of the present invention that are essential for function can be identified by methods known in the art, such as site-directed mutagenesis or alanine-scanning mutagenesis (Cunningham and Wells, Science 244:1081-1085 (1989)). The latter procedure introduces single alanine mutations for biological activity such as receptor binding or in vitro proliferative activity.

Of special interest are substitutions of charged amino acids with other charged or neutral amino acids which may produce proteins with highly desirable improved characteristics, such as less aggregation. Aggregation may not only reduce activity but also be problematic when preparing pharmaceutical formulations, because aggregates can be immunogenic (Pinckard, et al., Clin. Exp. Immunol. 2:331-340 (19674); Robbins, et al., Diabetes 36:838-845 (1987); Cleland, et al., Crit. Rev. Therapeutic Drug Carrier Systems 10:307-377 (1993)).

Since HFLP is a member of the Frizzled-related protein family, to modulate rather than completely eliminate biological activities of HFLP preferably mutations are made in sequences encoding amino acids in the HFLP conserved domain, i.e., in positions 6-126 of SEQ ID NO:2, more preferably in residues within this region which are not conserved in all members of the Frizzled-related protein family. Also forming part of the present invention are isolated polynucleotides comprising nucleic acid sequences which encode the above HFLP mutants.

Further preferred embodiments encompass polypeptide fragments comprising, or alternatively consisting of, the mature domain of HFLP (i.e., amino acid residues 1-328 of SEQ ID NO:2 and amino acid residues 1-325 of SEQ ID NO:4).

In specific embodiments, polypeptide fragments of the invention comprise, or alternatively, consist of, amino acid residues methionine-24 to glutamine-42; phenylalanine-57 to threonine-68; proline-75 to cysteine-83; histine-99 to threonine-126; aspartic acid-158 to threonine-168; asparagine-176 to lysine-186; cysteine-193 to lysine-207; and arginine-244 to lysine-264 of SEQ ID NO:2. These domains are also present in the corresponding positions of SEQ ID NO:4. These domains are regions of high identity identified between HFLP and bovine FRZB shown in FIG. 3.

The polypeptides of the present invention are preferably provided in an isolated form, and preferably are substantially purified. A recombinantly produced version of the HFLP polypeptide can be substantially purified by the one-step method described by Smith and Johnson (Gene 67:31-40 (1988)). Polypeptides of the invention also can be purified from natural or recombinant sources using anti-HFLP antibodies of the invention in methods which are well known in the art of protein purification.

The invention further provides an isolated HFLP polypeptide comprising an amino acid sequence selected from the group consisting of: (a) the amino acid sequence of the full-length HFLP polypeptide having the complete amino acid sequence shown in SEQ ID NO:4 (i.e., positions −43 to 325 of SEQ ID NO:4); (b) the amino acid sequence of the full-length HFLP polypeptide having the complete amino acid sequence shown in SEQ ID NO:4 (i.e., positions −42 to 325 of SEQ ID NO:4), excluding the N-terminal methionine; (c) the amino acid sequence of the mature HFLP polypeptide having the complete amino acid sequence in SEQ ID NO:4 (i.e., positions 1 to 325 of SEQ ID NO:4); (d) the amino acid sequence of the full-length HFLP polypeptide having the complete amino acid sequence shown in SEQ ID NO:2 (i.e., positions −19 to 328 of SEQ ID NO:2) plus the N-terminal 12 amino acid signal peptide from the bovine Frzb molecule; (e) the amino acid sequence of the full-length HFLP polypeptide having the complete amino acid sequence shown in SEQ ID NO:2 (i.e., positions −19 to 328 of SEQ ID NO:2) plus the N-terminal 12 amino acid signal peptide from the bovine Frzb molecule, excluding the N-terminal methionine; (f) the amino acid sequence of the HFLP polypeptide having the complete amino acid sequence in SEQ ID NO:2 (i.e., positions −19 to 328 of SEQ ID NO:2); (g) the amino acid sequence of the predicted mature HFLP polypeptide having the amino acid sequence at positions 1 to 328 in SEQ ID NO:2; (h) the amino acid sequence of the predicted frizzled domain of the HFLP polypeptide having the amino acid sequence in SEQ ID NO:2 (i.e., positions 6 to 126 of SEQ ID NO:2); (i) the amino acid sequence of the HFLP polypeptide having the complete amino acid sequence encoded by the cDNA clone contained in ATCC Deposit No. 209140; 0) the amino acid sequence of the mature HFLP polypeptide having the amino acid sequence encoded by the cDNA clone contained in ATCC Deposit No. 209140; and (k) the amino acid sequence of the frizzled domain of the HFLP polypeptide having the amino acid sequence encoded by the cDNA clone contained in ATCC Deposit No. 209140. The polypeptides of the present invention also include polypeptides having an amino acid sequence at least 80% identical, more preferably at least 90% identical, and still more preferably 95%, 96%, 97%, 98% or 99% identical to those described in (a), (b), (c), (d), (e), (f), (g), (h), (i), (j), or (k), above, as well as polypeptides having an amino acid sequence with at least 90% similarity, and more preferably at least 95% similarity, to those above.

Further polypeptides of the present invention include polypeptides which have at least 90% similarity, more preferably at least 95% similarity, and still more preferably at least 96%, 97%, 98% or 99% similarity to those described above. The polypeptides of the invention also comprise those which are at least 80% identical, more preferably at least 90% or 95% identical, still more preferably at least 96%, 97%, 98% or 99% identical to the polypeptide encoded by the deposited cDNA or to the polypeptide of SEQ ID NO:2 or to the polypeptide of SEQ ID NO:4, and also include portions of such polypeptides with at least 30 amino acids and more preferably at least 50 amino acids.

By “% similarity” for two polypeptides is intended a similarity score produced by comparing the amino acid sequences of the two polypeptides using the Bestfit program (Wisconsin Sequence Analysis Package, Version 8 for Unix, Genetics Computer Group, University Research Park, 575 Science Drive, Madison, Wis. 53711) and the default settings for determining similarity. Bestfit uses the local homology algorithm of Smith and Waterman (Advances in Applied Mathematics 2:482-489 (1981)) to find the best segment of similarity between two sequences.

By a polypeptide having an amino acid sequence at least, for example, 95% “identical” to a reference amino acid sequence of a HFLP polypeptide (the query sequence) is intended that the amino acid sequence of the polypeptide (the subject sequence) is identical to the reference sequence except that the polypeptide sequence may include up to five amino acid alterations per each 100 amino acids of the reference amino acid of the HFLP polypeptide. In other words, to obtain a polypeptide having an amino acid sequence at least 95% identical to a query amino acid sequence, up to 5% of the amino acid residues in the subject sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 90%, 95%, 96%, 97%, 98% or 99% identical to, for instance, the amino acid sequence shown in SEQ ID NO:2 or to the amino acid sequence shown in SEQ ID NO:4 or to the amino acid sequence encoded by deposited cDNA clone can be determined conventionally using known computer programs such the Bestfit program (Wisconsin Sequence Analysis Package, Version 8 for Unix, Genetics Computer Group, University Research Park, 575 Science Drive, Madison, Wis. 53711). A preferred method for determing the best overall match between a query sequence (a sequence of the present invention) and a subject sequence, also referred to as a global sequence alignment, can be determined using the FASTDB computer program based on the algorithm of Brutlag and colleagues (Comp. App. Biosci. (1990) 6:237-245). In a sequence alignment the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

If the subject sequence is shorter than the query sequence due to N- or C-terminal deletions, not because of internal deletions, a manual correction must be made to the results. This is becuase the FASTDB program does not account for N- and C-terminal truncations of the subject sequence when calculating global percent identity. For subject sequences truncated at the N- and C-termini, relative to the the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequnce are manually corrected for. No other manual corrections are to made for the purposes of the present invention.

The polypeptide of the present invention could be used as a molecular weight marker on SDS-PAGE gels or on molecular sieve gel filtration columns using methods well known to those of skill in the art.

As described in detail below, the polypeptides of the present invention can also be used to raise polyclonal and monoclonal antibodies, which are useful in assays for detecting HFLP protein expression as described below or as agonists and antagonists capable of enhancing or inhibiting HFLP protein function. Further, such polypeptides can be used in the yeast two-hybrid system to “capture” HFLP protein binding proteins which are also candidate agonists and antagonists according to the present invention. The yeast two hybrid system is described by Fields and Song (Nature 340:245-246 (1989)).

Epitope-Bearing Portions

In another aspect, the invention provides a peptide or polypeptide comprising an epitope-bearing portion of a polypeptide of the invention. The epitope of this polypeptide portion is an immunogenic or antigenic epitope of a polypeptide of the invention. An “immunogenic epitope” is defined as a part of a protein that elicits an antibody response when the whole protein is the immunogen. On the other hand, a region of a protein molecule to which an antibody can bind is defined as an “antigenic epitope.” The number of immunogenic epitopes of a protein generally is less than the number of antigenic epitopes (see, for instance, Geysen, et al., Proc. Natl. Acad. Sci. USA 81:3998-4002 (1983)).

As to the selection of peptides or polypeptides bearing an antigenic epitope (i.e., that contain a region of a protein molecule to which an antibody can bind), it is well known in that art that relatively short synthetic peptides that mimic part of a protein sequence are routinely capable of eliciting an antiserum that reacts with the partially mimicked protein (see, for instance, Sutcliffe, J. G., et al., Science 219:660-666 (1983)). Peptides capable of eliciting protein-reactive sera are frequently represented in the primary sequence of a protein, can be characterized by a set of simple chemical rules, and are confined neither to immunodominant regions of intact proteins (i.e., immunogenic epitopes) nor to the amino or carboxyl terminals. Antigenic epitope-bearing peptides and polypeptides of the invention are therefore useful to raise antibodies, including monoclonal antibodies, that bind specifically to a polypeptide of the invention (see, for instance, Wilson, et al., Cell 37:767-778 (1984)).

Antigenic epitope-bearing peptides and polypeptides of the invention preferably contain a sequence of at least seven, more preferably at least nine and most preferably between about 15 to about 30 amino acids contained within the amino acid sequence of a polypeptide of the invention. Non-limiting examples of antigenic polypeptides or peptides that can be used to generate HFLP-specific antibodies include: a polypeptide comprising amino acid residues from about Pro-1 to about Ala-9, from about Met-32 to about Ile-40, from about Asn-45 to about Glu-53, from about Gln-61 to about Asn-69, from about Pro-94 to about Cys-102, from about Arg-104 to about Leu-112, from about Tyr-132 to about Pro-140, from about Asp-146 to about Ile-154, from about Pro-158 to about Pro-166, from about Asp-170 to about Arg-178, from about Lys-180 to about Leu-188, from about Gln-208 to about Thr-216, from about Ser-227 to about Gln-235, from about Trp-277 to about Gln-287, from about Thr-305 to about Pro-314, from about Ser-312 to about Lys-320, from about Thr-321 to about Pro-329, from about Thr-305 to about Lys-320, from about Pro-314 to about Pro-329, from about Lys-303 to about Asn-332, from about Gln-339 to about Val-347. These polypeptide fragments have been determined to bear antigenic epitopes of the HFLP protein by the analysis of the Jameson-Wolf antigenic index, as shown in FIGS. 4 and 5, above.

The epitope-bearing peptides and polypeptides of the invention may be produced by any conventional means (see, for example, Houghten, R. A., et al., Proc. Natl. Acad. Sci. USA 82:5131-5135 (1985); and U.S. Pat. No. 4,631,211 to Houghten, et al. (1986)).

Epitope-bearing peptides and polypeptides of the invention are used to induce antibodies according to methods well known in the art (see, for instance, Sutcliffe, et al., supra; Wilson, et al., supra; Chow, M., et al., Proc. Natl. Acad. Sci. USA 82:910-914; and Bittle, F. J., et al., J. Gen. Virol. 66:2347-2354 (1985)). Immunogenic epitope-bearing peptides of the invention, i.e., those parts of a protein that elicit an antibody response when the whole protein is the immunogen, are identified according to methods known in the art (see, for instance, Geysen, et al., supra). Further still, U.S. Pat. No. 5,194,392, issued to Geysen, describes a general method of detecting or determining the sequence of monomers (amino acids or other compounds) which is a topological equivalent of the epitope (i.e., a “mimotope”) which is complementary to a particular paratope (antigen binding site) of an antibody of interest. More generally, U.S. Pat. No. 4,433,092, issued to Geysen, describes a method of detecting or determining a sequence of monomers which is a topographical equivalent of a ligand which is complementary to the ligand binding site of a particular receptor of interest. Similarly, U.S. Pat. No. 5,480,971, issued to Houghten and colleagues, on Peralkylated Oligopeptide Mixtures discloses linear C1-C7-alkyl peralkylated oligopeptides and sets and libraries of such peptides, as well as methods for using such oligopeptide sets and libraries for determining the sequence of a peralkylated oligopeptide that preferentially binds to an acceptor molecule of interest. Thus, non-peptide analogs of the epitope-bearing peptides of the invention also can be made routinely by these methods.

Fusion Proteins

As one of skill in the art will appreciate, HFLP polypeptides of the present invention and the epitope-bearing fragments thereof described above can be combined with parts of the constant domain of immunoglobulins (IgG), resulting in chimeric polypeptides. These fusion proteins facilitate purification and show an increased half-life in vivo. This has been shown, e.g., for chimeric proteins consisting of the first two domains of the human CD4-polypeptide and various domains of the constant regions of the heavy or light chains of mammalian immunoglobulins (EP A 394,827; Traunecker, et al., Nature 331:84-86 (1988)). Fusion proteins that have a disulfide-linked dimeric structure due to the IgG part can also be more efficient in binding and neutralizing other molecules than the monomeric HFLP protein or protein fragment alone (Fountoulakis, et al., J. Biochem. 270:3958-3964 (1995)).

Functional Activities

The functional activity of HFLP polypeptides, and fragments, variants derivatives, and analogs thereof, can be assayed by various methods.

For example, in one embodiment where one is assaying for the ability to bind or compete with full-length HFLP polypeptide for binding to anti-HFLP antibody, various immunoassays known in the art can be used, including but not limited to, competitive and non-competitive assay systems using techniques such as radioimmunoassays, ELISA (enzyme linked immunosorbent assay), “sandwich” immunoassays, immunoradiometric assays, gel diffusion precipitation reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

In another embodiment, where an HFLP-ligand is identified, binding can be assayed, e.g., by means well-known in the art. In another embodiment, physiological correlates of HFLP binding to its substrates (signal transduction) can be assayed.

Other methods to measure the ability of HFLP polypeptides and fragments, variants derivatives and analogs thereof to elicit HFLP-related biological activity will be known to the skilled artisan and are within the scope of the invention.

Antibodies

HFLP protein-specific antibodies for use in the present invention can be raised against the intact HFLP protein or an antigenic polypeptide fragment thereof, which may be presented together with a carrier protein, such as an albumin, to an animal system (such as rabbit or mouse) or, if it is long enough (at least about 25 amino acids), without a carrier.

As used herein, the term “antibody” (Ab) or “monoclonal antibody” (Mab) is meant to include intact molecules as well as antibody fragments (such as, for example, Fab and F(ab′)₂ fragments) which are capable of specifically binding to HFLP protein. Fab and F(ab′)₂ fragments lack the Fc fragment of intact antibody, clear more rapidly from the circulation, and may have less non-specific tissue binding of an intact antibody (Wahl, et al., J. Nucl. Med. 24:316-325 (1983)). Thus, these fragments are preferred.

The antibodies of the present invention may be prepared by any of a variety of methods. For example, cells expressing the HFLP protein or an antigenic fragment thereof can be administered to an animal in order to induce the production of sera containing polyclonal antibodies. In a preferred method, a preparation of HFLP protein is prepared and purified to render it substantially free of natural contaminants. Such a preparation is then introduced into an animal in order to produce polyclonal antisera of greater specific activity.

In the most preferred method, the antibodies of the present invention are monoclonal antibodies (or HFLP protein binding fragments thereof). Such monoclonal antibodies can be prepared using hybridoma technology (Kohler, et al., Nature 256:495 (1975); Kohler, et al., Eur. J. Immunol. 6:511 (1976); Kohler, et al., Eur. J. Immunol. 6:292 (1976); Hammerling, et al., in: Monoclonal Antibodies and T-Cell Hybridomas, Elsevier, N.Y., (1981) pp. 563-681)). In general, such procedures involve immunizing an animal (preferably a mouse) with a HFLP protein antigen or, more preferably, with a HFLP protein-expressing cell. Suitable cells can be recognized by their capacity to bind anti-HFLP protein antibody. Such cells may be cultured in any suitable tissue culture medium; however, it is preferable to culture cells in Earle's modified Eagle's medium supplemented with 10% fetal bovine serum (inactivated at about 56° C.), and supplemented with about 10 μg/l of nonessential amino acids, about 1,000 U/ml of penicillin, and about 100 μg/ml of streptomycin. The splenocytes of such mice are extracted and fused with a suitable myeloma cell line. Any suitable myeloma cell line may be employed in accordance with the present invention; however, it is preferable to employ the parent myeloma cell line (SP2O), available from the American Type Culture Collection, 10801 University Boulevard, Manassas, Va. 20110-2209. After fusion, the resulting hybridoma cells are selectively maintained in HAT medium, and then cloned by limiting dilution as described by Wands and colleagues (Gastroenterology 80:225-232 (1981)). The hybridoma cells obtained through such a selection are then assayed to identify clones which secrete antibodies capable of binding the HFLP protein antigen.

Alternatively, additional antibodies capable of binding to the HFLP protein antigen may be produced in a two-step procedure through the use of anti-idiotypic antibodies. Such a method makes use of the fact that antibodies are themselves antigens, and that, therefore, it is possible to obtain an antibody which binds to a second antibody. In accordance with this method, HFLP protein-specific antibodies are used to immunize an animal, preferably a mouse. The splenocytes of such an animal are then used to produce hybridoma cells, and the hybridoma cells are screened to identify clones which produce an antibody whose ability to bind to the HFLP protein-specific antibody can be blocked by the HFLP protein antigen. Such antibodies comprise anti-idiotypic antibodies to the HFLP protein-specific antibody and can be used to immunize an animal to induce formation of further HFLP protein-specific antibodies.

It will be appreciated that Fab and F(ab′)2 and other fragments of the antibodies of the present invention may be used according to the methods disclosed herein. Such fragments are typically produced by proteolytic cleavage, using enzymes such as papain (to produce Fab fragments) or pepsin (to produce F(ab′)₂ fragments). Alternatively, HFLP protein-binding fragments can be produced through the application of recombinant DNA technology or through synthetic chemistry.

For in vivo use of anti-HFLP in humans, it may be preferable to use “humanized” chimeric monoclonal antibodies. Such antibodies can be produced using genetic constructs derived from hybridoma cells producing the monoclonal antibodies described above. Methods for producing chimeric antibodies are known in the art (Morrison, Science 229:1202 (1985); Oi, et al., BioTechniques 4:214 (1986); Cabilly, et al., U.S. Pat. No. 4,816,567; Taniguchi, et al., EP 171496; Morrison, et al., EP 173494; Neuberger, et al., WO 8601533; Robinson, et al., WO 8702671; Boulianne, et al., Nature 312:643 (1984); Neuberger, et al., Nature 314:268 (1985).

Cell Growth and Differentiation-Related Disorders Diagnosis

The present inventors have discovered that HFLP is expressed not only in cells obtained from a Hodgkin's Lymphoma, but also in adipose tissue, fetal lung, atrophic endometrium, synovial fibroblasts, synovial hypoxia, chondrosarcoma, pancreatic tumor, ovary, osteoclastoma, menijioma, hepatocellular tumor, IL-1- and TNF-stimulated synovial cells, osteoblasts, uterus, striatum depression, chronic synovitis, substantia nigra, spinal cord, testes, placenta, and whole 8 week old human embryo. For a number of cell growth and differentiation-related disorders, substantially altered (increased or decreased) levels of HFLP gene expression can be detected in many tissues or cells or bodily fluids (e.g., sera, plasma, urine, synovial fluid or spinal fluid) taken from an individual having such a disorder, relative to a “standard” HFLP gene expression level, that is, the HFLP expression level in a given tissue or bodily fluid from an individual not having the cell growth and differentiation disorder. Thus, the invention provides a diagnostic method useful during diagnosis of a cell growth and differentiation disorder, which involves measuring the expression level of the gene encoding the HFLP protein in a given tissue or cell or bodily fluid from an individual and comparing the measured gene expression level with a standard HFLP gene expression level, whereby an increase or decrease in the gene expression level compared to the standard is indicative of a cell growth and differentiation disorder.

In particular, it is believed that certain tissues in mammals with any of a variety of cancers, including bone and breast cancers, express significantly reduced levels of the HFLP protein and mRNA encoding the HFLP protein when compared to a corresponding “standard” level. Further, it is believed that enhanced levels of the HFLP protein can be detected in certain body fluids (e.g., sera, plasma, urine, and spinal fluid) from mammals with such a cancer when compared to sera from mammals of the same species not having the cancer.

Thus, the invention provides a diagnostic method useful during diagnosis of a cell growth and differentiation disorder, including cancers, which involves measuring the expression level of the gene encoding the HFLP protein in a given tissue or cell or body fluid from an individual and comparing the measured gene expression level with a standard HFLP gene expression level, whereby an increase or decrease in the gene expression level compared to the standard is indicative of a cell growth and differentiation disorder.

Where a diagnosis of a disorder in the regulation of cell growth and differentiation, including diagnosis of a tumor, has already been made according to conventional methods, the present invention is useful as a prognostic indicator, whereby patients exhibiting enhanced or depressed HFLP gene expression will experience a worse clinical outcome relative to patients expressing the gene at a level nearer the standard level.

By “assaying the expression level of the gene encoding the HFLP protein” is intended qualitatively or quantitatively measuring or estimating the level of the HFLP protein or the level of the mRNA encoding the HFLP protein in a first biological sample either directly (e.g., by determining or estimating absolute protein level or mRNA level) or relatively (e.g., by comparing to the HFLP protein level or mRNA level in a second biological sample). Preferably, the HFLP protein level or mRNA level in the first biological sample is measured or estimated and compared to a standard HFLP protein level or mRNA level, the standard being taken from a second biological sample obtained from an individual not having the disorder or being determined by averaging levels from a population of individuals not having a disorder of the regulation of cell growth and regulation. As will be appreciated in the art, once a standard HFLP protein level or mRNA level is known, it can be used repeatedly as a standard for comparison.

By “biological sample” is intended any biological sample obtained from an individual, body fluid, cell line, tissue culture, or other source which contains HFLP protein or mRNA. As indicated, biological samples include body fluids (such as sera, plasma, urine, synovial fluid and spinal fluid) which contain free HFLP protein, tissues or cells which can be characterized by a disorder of the regulation of cell growth and regulation, and other tissue sources found to express complete or mature HFLP or an HFLP receptor. Methods for obtaining tissue biopsies and body fluids from mammals are well known in the art. Where the biological sample is to include mRNA, a tissue biopsy is the preferred source.

The present invention is useful for diagnosis or treatment of various cell growth and differentiation-related disorders in mammals, preferably humans. Such disorders include tumors, cancers, interstitial lung disease (such as Langerhans cell granulomatosis, and any disregulation of cell function including, but not limited to, autoimmunity, arthritis, leukemias, lymphomas, immunosuppression, immunity, humoral immunity, inflammatory bowel disease, myelosuppression, and the like.

Total cellular RNA can be isolated from a biological sample using any suitable technique such as the single-step guanidinium-thiocyanate-phenol-chloroform method described by Chomczynski and Sacchi (Anal. Biochem. 162:156-159 (1987)). Levels of mRNA encoding the HFLP protein are then assayed using any appropriate method. These include Northern blot analysis, S1 nuclease mapping, the polymerase chain reaction (PCR), reverse transcription in combination with the polymerase chain reaction (RT-PCR), and reverse transcription in combination with the ligase chain reaction (RT-LCR).

Assaying HFLP protein levels in a biological sample can occur using antibody-based techniques. For example, HFLP protein expression in tissues can be studied with classical immunohistological methods (Jalkanen, M., et al., J. Cell. Biol. 101:976-985 (1985); Jalkanen, M., et al., J. Cell. Biol. 105:3087-3096 (1987)). Other antibody-based methods useful for detecting HFLP protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes, such as iodine (¹²⁵I, ¹²¹I), carbon (¹⁴C), sulfur (³⁵S), tritium (³H), indium (¹¹²In), and technetium (^{99m}Tc), and fluorescent labels, such as fluorescein and rhodamine, and biotin.

In addition to assaying HFLP protein levels in a biological sample obtained from an individual, HFLP protein can also be detected in vivo by imaging. Antibody labels or markers for in vivo imaging of HFLP protein include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the antibody by labeling of nutrients for the relevant hybridoma.

A HFLP protein-specific antibody or antibody fragment which has been labeled with an appropriate detectable imaging moiety, such as a radioisotope (for example, ³¹¹I, ¹¹²In, ^{99m}Tc), a radio-opaque substance, or a material detectable by nuclear magnetic resonance, is introduced (for example, parenterally, subcutaneously or intraperitoneally) into the mammal to be examined for immune system disorder. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ^{99m}Tc. The labeled antibody or antibody fragment will then preferentially accumulate at the location of cells which contain HFLP protein. In vivo tumor imaging is described by Burchiel and coworkers (Chapter 13 in Tumor Imaging: The Radiochemical Detection of Cancer, Burchiel, S. W. and Rhodes, B. A., eds., Masson Publishing Inc. (1982)).

Treatment

As noted above, HFLP polynucleotides and polypeptides are useful for diagnosis of conditions involving abnormally high or low expression of HFLP activities. Given the cells and tissues where HFLP is expressed as well as the activities modulated by HFLP, it is readily apparent that a substantially altered (increased or decreased) level of expression of HFLP in an individual compared to the standard or “normal” level produces pathological conditions related to the bodily system(s) in which HFLP is expressed and/or is active.

It will also be appreciated by one of ordinary skill that, since the HFLP protein of the invention is a member of the frizzled polypeptide family, the mature secreted form of the protein may be released in soluble form from the cells which express the HFLP by proteolytic cleavage. Therefore, when HFLP mature form is added from an exogenous source to cells, tissues or the body of an individual, the protein will exert its physiological activities on its target cells of that individual.

Therefore, it will be appreciated that conditions caused by a decrease in the standard or normal level of HFLP activity in an individual, particularly disorders of the regulation of cell growth and regulation, can be treated by administration of HFLP polypeptide (in the form of the mature protein). Thus, the invention also provides a method of treatment of an individual in need of an increased level of HFLP activity comprising administering to such an individual a pharmaceutical composition comprising an amount of an isolated HFLP polypeptide of the invention, particularly a mature form of the HFLP protein of the invention, effective to increase the HFLP activity level in such an individual.

Since HFLP is expressed in a wide variety of cell and tissue types, it is expected that it may have a wide range of therapeutic activities. HFLP functions by antagonistically interfering with a stimulatory binding of a Wnt family member or similar factor to a frizzled-like receptor molecule or other corresponding receptor molecule. The HFLP factor functions by binding to the soluble Wnt family member and thereby preventing its binding to the corresponding receptor. The result is the prevention of stimulation of the corresponding signal transduction pathway. Such a dimunition of the signal in a signal transduction pathway results in a corresponding reduction in signal for cellular activation and ultimate growth and division. As a result, HFLP proteins and muteins thereof may be useful therapeutically as a means of reducing a signal for one of a variety of cell types to become activated and to divide. Such a therapeutic regeime is especially useful for cells which have lost control over regulation of cellular division, i.e. have entered the cancerous state. In this case, it will be of use to exploit this capability of HFLP or a mutein thereof to effectively antagonize the signal causing the cell to divide. Such a scenario may be particularly useful in the therapeutic control of a variety of cancers including bone, breast, colon, lymphomas, leukemias, epithelial carcinomas, pancreatic, stomach, liver, lung, melanoma, prostate, ovarian, uterine, bladder, gliomas, retinoblastomas, sarcomas, and the like.

HFLP and muteins thereof may also be employed to regulate hematopoiesis, by regulating the activation and differentiation of various hematopoietic progenitor cells, for example, to release mature leukocytes from the bone marrow following chemotherapy, i.e., in stem cell mobilization.

Furthermore, the activation of macrophages and their precursors, and of neutrophils, basophils, B lymphocytes and some T-cell subsets, e.g., activated and CD8 cytotoxic T cells and natural killer cells, in certain auto-immune and chronic inflammatory and infective diseases may also be reduced through the action of HFLP and muteins thereof. The result of blocking such activation will be a decrease in the degenerative and degradative effects of such chronic inflammatory and auto-immune disease states. Examples of such auto-immune diseases include multiple sclerosis, and insulin-dependent diabetes.

HFLP may also be employed to treat infectious diseases including silicosis, sarcoidosis, idiopathic pulmonary fibrosis by preventing the activation of mononuclear phagocytes. It may also be employed to treat idiopathic hyper-eosinophilic syndrome by preventing eosinophil production. HFLP may also be employed to treat histamine-mediated allergic reactions and immunological disorders including late phase allergic reactions, chronic urticaria, and atopic dermatitis by inhibiting activation of mast cells and basophils and the resulting degranulation and release of histamine from such activated cells. IgE-mediated allergic reactions such as allergic asthma, rhinitis, and eczema may also be treated. HFLP may also be employed to treat chronic and acute inflammation by preventing the activation of monocytes recruited to a wound area. It may also be employed to regulate normal pulmonary macrophage populations, since chronic and acute inflammatory pulmonary diseases are associated with sequestration and activation of mononuclear phagocytes in the lung. HFLP may also be employed to treat rheumatoid arthritis by preventing the activation of monocytes recruited into synovial fluid in the joints of patients. Monocyte activation plays a significant role in the pathogenesis of both degenerative and inflammatory arthropathies. HFLP may be employed to interfere with the deleterious cascades attributed primarily to IL-1 and TNF, which prevents the biosynthesis of other inflammatory cytokines. In this way, HFLP may be employed to prevent inflammation. HFLP may also be employed to treat cases of bone marrow failure, for example, aplastic anemia and myelodysplastic syndrome. HFLP may also be employed to treat asthma and allergy by preventing eosinophil activation in the lung. HFLP may also be employed to treat subepithelial basement membrane fibrosis which is a prominent feature of the asthmatic lung.

Formulations

The HFLP polypeptide composition will be formulated and dosed in a fashion consistent with good medical practice, taking into account the clinical condition of the individual patient (especially the side effects of treatment with HFLP polypeptide alone), the site of delivery of the HFLP polypeptide composition, the method of administration, the scheduling of administration, and other factors known to practitioners. The “effective amount” of HFLP polypeptide for purposes herein is thus determined by such considerations.

As a general proposition, the total pharmaceutically effective amount of HFLP polypeptide administered parenterally per dose will be in the range of about 1 μg/kg/day to 10 mg/kg/day of patient body weight, although, as noted above, this will be subject to therapeutic discretion. More preferably, this dose is at least 0.01 mg/kg/day, and most preferably for humans between about 0.01 and 1 mg/kg/day for the hormone. If given continuously, the HFLP polypeptide is typically administered at a dose rate of about 1 μg/kg/hour to about 50 μg/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect.

Pharmaceutical compositions containing the HFLP of the invention may be administered orally, rectally, parenterally, intracistemally, intravaginally, intraperitoneally, topically (as by powders, ointments, drops or transdermal patch), bucally, or as an oral or nasal spray. By “pharmaceutically acceptable carrier” is meant a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion.

The HFLP polypeptide is also suitably administered by sustained-release systems. Suitable examples of sustained-release compositions include semi-permeable polymer matrices in the form of shaped articles, e.g., films, or mirocapsules. Sustained-release matrices include polylactides (U.S. Pat. No. 3,773,919, EP 58,481), copolymers of L-glutamic acid and gamma-ethyl-L-glutamate (Sidman, U., et al., Biopolymers 22:547-556 (1983)), poly (2-hydroxyethyl methacrylate; Langer, R., et al., J. Biomed. Mater. Res. 15:167-277 (1981), and Langer, R., Chem. Tech. 12:98-105 (1982)), ethylene vinyl acetate (Langer, R., et al., Id.) or poly-D-(−)-3-hydroxybutyric acid (EP 133,988). Sustained-release HFLP polypeptide compositions also include liposomally entrapped HFLP polypeptide. Liposomes containing HFLP polypeptide are prepared by methods known in the art (DE 3,218,121; Epstein, et al., Proc. Natl. Acad. Sci. (USA) 82:3688-3692 (1985); Hwang, et al., Proc. Natl. Acad. Sci. (USA) 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324). Ordinarily, the liposomes are of the small (about 200-800 Angstroms) unilamellar type in which the lipid content is greater than about 30 mol. percent cholesterol, the selected proportion being adjusted for the optimal HFLP polypeptide therapy.

For parenteral administration, in one embodiment, the HFLP polypeptide is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to polypeptides.

Generally, the formulations are prepared by contacting the HFLP polypeptide uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

The HFLP polypeptide is typically formulated in such vehicles at a concentration of about 0.1 mg/ml to 100 mg/ml, preferably 1-10 mg/ml, at a pH of about 3 to 8. It will be understood that the use of certain of the foregoing excipients, carriers, or stabilizers will result in the formation of HFLP polypeptide salts.

HFLP polypeptide to be used for therapeutic administration must be sterile. Sterility is readily accomplished by filtration through sterile filtration membranes (e.g., 0.2 micron membranes). Therapeutic HFLP polypeptide compositions generally are placed into a container having a sterile access port, for example, an intravenous solution bag or vial having a stopper pierceable by a hypodermic injection needle.

HFLP polypeptide ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous HFLP polypeptide solution, and the resulting mixture is lyophilized. The infusion solution is prepared by reconstituting the lyophilized HFLP polypeptide using bacteriostatic water-for-injection (WFI).

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, the polypeptides of the present invention may be employed in conjunction with other therapeutic compounds.

Agonists and Antagonists—Assays and Molecules

The invention also provides a method of screening compounds to identify those which enhance or block the action of HFLP on cells, such as its interaction with HFLP-binding molecules such as receptor molecules. An agonist is a compound which increases the natural biological functions of HFLP or which functions in a manner similar to HFLP, while antagonists decrease or eliminate such functions.

In another aspect of this embodiment the invention provides a method for identifying a receptor protein or other protein which binds specifically to an HFLP polypeptide. For example, a cellular compartment, such as a membrane or a preparation thereof, may be prepared from a cell that expresses a molecule that binds HFLP. The preparation is incubated with labeled HFLP and complexes of HFLP bound to the receptor or other binding protein are isolated and characterized according to routine methods known in the art. Alternatively, the HFLP polypeptide may be bound to a solid support so that binding molecules solubilized or secreted from cells are bound to the column and then eluted and characterized according to routine methods.

In the assay of the invention for agonists or antagonists, a cellular compartment, such as a membrane or a preparation thereof, may be prepared from a cell that expresses a molecule that binds HFLP, such as a molecule of a signaling or regulatory pathway modulated by HFLP. The preparation is incubated with labeled HFLP in the absence or the presence of a candidate molecule which may be an HFLP agonist or antagonist. The ability of the candidate molecule to bind the binding molecule is reflected in decreased binding of the labeled ligand. Molecules which bind gratuitously, i.e., without inducing the effects of HFLP on binding the HFLP binding molecule, are most likely to be good antagonists. Molecules that bind well and elicit effects that are the same as or closely related to HFLP are agonists.

HFLP-like effects of potential agonists and antagonists may by measured, for instance, by determining activity of a second messenger system following interaction of the candidate molecule with a cell or appropriate cell preparation, and comparing the effect with that of HFLP or molecules that elicit the same effects as HFLP. Second messenger systems that may be useful in this regard include but are not limited to AMP guanylate cyclase, ion channel or phosphoinositide hydrolysis second messenger systems.

Another example of an assay for HFLP antagonists is a competitive assay that combines HFLP and a potential antagonist with membrane-bound HFLP receptor molecules or recombinant HFLP receptor molecules under appropriate conditions for a competitive inhibition assay. HFLP can be labeled, such as by radioactivity, such that the number of HFLP molecules bound to a receptor molecule can be determined accurately to assess the effectiveness of the potential antagonist.

Potential antagonists include small organic molecules, peptides, polypeptides and antibodies that bind to a polypeptide of the invention and thereby inhibit or extinguish its activity. Potential antagonists also may be small organic molecules, a peptide, a polypeptide such as a closely related protein or antibody that binds the same sites on a binding molecule, such as a receptor molecule, without inducing HFLP-induced activities, thereby preventing the action of HFLP by excluding HFLP from binding.

Other potential antagonists include antisense molecules. Antisense technology can be used to control gene expression through antisense DNA or RNA or through triple-helix formation. Antisense techniques are discussed in a number of studies (for example, Okano, J. Neurochem. 56:560 (1991); “Oligodeoxynucleotides as Antisense Inhibitors of Gene Expression.” CRC Press, Boca Raton, Fla. (1988)). Triple helix formation is discussed in a number of studies, as well (for instance, Lee, et al., Nucleic Acids Research 6:3073 (1979); Cooney, et al., Science 241:456 (1988); Dervan, et al., Science 251:1360 (1991)). The methods are based on binding of a polynucleotide to a complementary DNA or RNA. For example, the 5′ coding portion of a polynucleotide that encodes the mature polypeptide of the present invention may be used to design an antisense RNA oligonucleotide of from about 10 to 40 base pairs in length. A DNA oligonucleotide is designed to be complementary to a region of the gene involved in transcription thereby preventing transcription and the production of HFLP. The antisense RNA oligonucleotide hybridizes to the mRNA in vivo and blocks translation of the mRNA molecule into HFLP polypeptide. The oligonucleotides described above can also be delivered to cells such that the antisense RNA or DNA may be expressed in vivo to inhibit production of HFLP protein.

The agonists and antagonists may be employed in a composition with a pharmaceutically acceptable carrier, e.g., as described above.

The antagonists may be employed for instance to inhibit the activity of endogeneous HFLP. Such an inhibition may be beneficial in a number of situations including the regulation of hematopoiesis. Targeted hematopoietic regulation may be achieved by regulating the activation and differentiation of various hematopoietic progenitor cells, for example, to release mature leukocytes from the bone marrow following chemotherapy, i.e., in stem cell mobilization.

In addition, antagonists of HFLP, may be used to promote wound healing. In such a situation, wound healing will be promoted by blocking the anti-proliferative effects that HFLP and muteins thereof may elicit on a cell or in a tissue. For example, wound healing may be accelerated by an HFLP antagonist by releasing the regulatory influence of HFLP on an epithelial cell by preventing the binding of HFLP to the Wnt family of proteins. By preventing the HFLP-Wnt interaction, Wnt proteins are then free to bind to the appropriate frizzled-like receptors, stimulate the corresponding signal transduction pathways, promote cellular activation and division, and, ultimately, promote accelerated wound healing.

Antibodies against HFLP may be employed to bind to and inhibit HFLP activity to treat any cellular growth and differentiation disorder attributable to an undesirable interaction of HFLP with a Wnt family or related protein resulting in a reduced interaction of the Wnt family or other protein with its native receptor molecule. Any of the above antagonists may be employed in a composition with a pharmaceutically acceptable carrier, e.g., as hereinafter described.

Gene Mapping

The nucleic acid molecules of the present invention are also valuable for chromosome identification. The sequence is specifically targeted to and can hybridize with a particular location on an individual human chromosome. Moreover, there is a current need for identifying particular sites on the chromosome. Few chromosome marking reagents based on actual sequence data (repeat polymorphisms) are presently available for marking chromosomal location. The mapping of DNAs to chromosomes according to the present invention is an important first step in correlating those sequences with genes associated with disease.

In certain preferred embodiments in this regard, the cDNA herein disclosed is used to clone genomic DNA of a HFLP protein gene. This can be accomplished using a variety of well known techniques and libraries, which generally are available commercially. The genomic DNA then is used for in situ chromosome mapping using well known techniques for this purpose.

In addition, in some cases, sequences can be mapped to chromosomes by preparing PCR primers (preferably 15-25 bp) from the cDNA. Computer analysis of the 3′ untranslated region of the gene is used to rapidly select primers that do not span more than one exon in the genomic DNA, thus complicating the amplification process. These primers are then used for PCR screening of somatic cell hybrids containing individual human chromosomes. Fluorescence in situ hybridization (“FISH”) of a cDNA clone to a metaphase chromosomal spread can be used to provide a precise chromosomal location in one step. This technique can be used with probes from the cDNA as short as 50 or 60 bp (for a review of this technique, see Verma, et al., Human Chromosomes: A Manual Of Basic Techniques, Pergamon Press, New York (1988)).

Once a sequence has been mapped to a precise chromosomal location, the physical position of the sequence on the chromosome can be correlated with genetic map data. Such data are found, for example, on the World Wide Web (McKusick, V. Mendelian Inheritance In Man, available on-line through Johns Hopkins University, Welch Medical Library). The relationship between genes and diseases that have been mapped to the same chromosomal region are then identified through linkage analysis (coinheritance of physically adjacent genes).

Next, it is necessary to determine the differences in the cDNA or genomic sequence between affected and unaffected individuals. If a mutation is observed in some or all of the affected individuals but not in any normal individuals, then the mutation is likely to be the causative agent of the disease.

Having generally described the invention, the same will be more readily understood by reference to the following examples, which are provided by way of illustration and are not intended as limiting.

### EXAMPLES

Examples 1-3, as follows, are directed towards the use of HFLP encoded by the deposited cDNA clone, with the sequence as shown in FIGS. 1A and 1B (SEQ ID NO:1). One of ordianary skill in the art will immediately recognize the fact that HFLP as shown in FIGS. 2A and 2B (SEQ ID NO:3) may be practiced as put forth in the following Examples 1-3 simply be designing similar oligonucleotide primers based on the sequence presented as SEQ ID NO:3 rather than that presented as SEQ ID NO:1. As such, the following Examples 1-3 may be used to practice not only the HFLP of the invention as encoded by the deposited cDNA clone or as shown in FIGS. 1A and 1B (SEQ ID NO:1), but also the HFLP of the invention as shown in FIGS. 2A and 2B (SEQ ID NO:3).

### Example 1(a)

### Expression and Purification of “His-tagged” HFLP in E. coli

The bacterial expression vector pQE60 is used for bacterial expression in this example. (QIAGEN, Inc., 9259 Eton Avenue, Chatsworth, Calif., 91311). pQE60 encodes ampicillin antibiotic resistance (“Ampr”) and contains a bacterial origin of replication (“ori”), an IPTG inducible promoter, a ribosome binding site (“RBS”), six codons encoding histidine residues that allow affinity purification using nickel-nitrilo-tri-acetic acid (“Ni-NTA”) affinity resin (QIAGEN, Inc., supra) and suitable single restriction enzyme cleavage sites. These elements are arranged such that an inserted DNA fragment encoding a polypeptide expresses that polypeptide with the six His residues (i.e., a “6×His tag”) covalently linked to the carboxyl terminus of that polypeptide.

The DNA sequence encoding the desired portion HFLP protein lacking the hydrophobic leader sequence is amplified from the deposited cDNA clone using PCR oligonucleotide primers which anneal to the amino terminal sequences of the desired portion of the HFLP protein and to sequences in the deposited construct 3′ to the cDNA coding sequence. Additional nucleotides containing restriction sites to facilitate cloning in the pQE60 vector are added to the 5′ and 3′ sequences, respectively.

For cloning the mature protein, the 5′ primer has the sequence 5′ GACTCCATGGGCGTGCGCGGCGCGCCC 3′ (SEQ ID NO:12) containing the underlined Nco I restriction site followed by 17 nucleotides of the amino terminal coding sequence of the mature HFLP sequence in SEQ ID NO:2. One of ordinary skill in the art would appreciate, of course, that the point in the protein coding sequence where the 5′ primer begins may be varied to amplify a DNA segment encoding any desired portion of the complete protein shorter or longer than the mature form. The 3′ primer has the sequence 5′ GACTGGATCCCACTCTTTTCGGGTTTGTTC 3′ (SEQ ID NO:13) containing the underlined Bam HI restriction site followed by 20 nucleotides complementary to the 3′ end of the coding sequence immediately before the stop codon in the HFLP DNA sequence in FIGS. 1A and 1B, with the coding sequence aligned with the restriction site so as to maintain its reading frame with that of the six His codons in the pQE60 vector.

The amplified HFLP DNA fragment and the vector pQE60 are digested with Nco I and Bam HI and the digested DNAs are then ligated together. Insertion of the HFLP DNA into the restricted pQE60 vector places the HFLP protein coding region downstream from the IPTG-inducible promoter and in-frame with an initiating AUG and the six histidine codons.

The ligation mixture is transformed into competent E. coli cells using standard procedures such as those described by Sambrook and colleagues (Molecular Cloning: a Laboratory Manual, 2nd Ed.; Cold Spring Harbor Laboratory Press, Cold Spring Harbor, N.Y. (1989)). E. coli strain M15/rep4, containing multiple copies of the plasmid pREP4, which expresses the lac repressor and confers kanamycin resistance (“Kan^{R}”), is used in carrying out the illustrative example described herein. This strain, which is only one of many that are suitable for expressing HFLP protein, is available commercially (QIAGEN, Inc., supra). Transformants are identified by their ability to grow on LB plates in the presence of ampicillin and kanamycin. Plasmid DNA is isolated from resistant colonies and the identity of the cloned DNA confirmed by restriction analysis, PCR and DNA sequencing.

Clones containing the desired constructs are grown overnight (“O/N”) in liquid culture in LB media supplemented with both ampicillin (100 μg/ml) and kanamycin (25 μg/ml). The O/N culture is used to inoculate a large culture, at a dilution of approximately 1:25 to 1:250. The cells are grown to an optical density at 600 nm (“OD600”) of between 0.4 and 0.6. Isopropyl-β-D-thiogalactopyranoside (“IPTG”) is then added to a final concentration of 1 mM to induce transcription from the lac repressor sensitive promoter, by inactivating the lacI repressor. Cells subsequently are incubated further for 3 to 4 hours. Cells then are harvested by centrifugation.

The cells are then stirred for 3-4 hours at 4° C. in 6M guanidine-HCl, pH 8. The cell debris is removed by centrifugation, and the supernatant containing the HFLP is loaded onto a nickel-nitrilo-tri-acetic acid (“Ni-NTA”) affinity resin column (QIAGEN, Inc., supra). Proteins with a 6×His tag bind to the Ni-NTA resin with high affinity and can be purified in a simple one-step procedure (for details see: The QIAexpressionist, 1995, QIAGEN, Inc., supra). Briefly the supernatant is loaded onto the column in 6 M guanidine-HCl, pH 8, the column is first washed with 10 volumes of 6 M guanidine-HCl, pH 8, then washed with 10 volumes of 6 M guanidine-HCl pH 6, and finally the HFLP is eluted with 6 M guanidine-HCl, pH 5.

The purified protein is then renatured by dialyzing it against phosphate-buffered saline (PBS) or 50 mM Na-acetate, pH 6 buffer plus 200 mM NaCl. Alternatively, the protein can be successfully refolded while immobilized on the Ni-NTA column. The recommended conditions are as follows: renature using a linear 6M-1M urea gradient in 500 mM NaCl, 20% glycerol, 20 mM Tris/HCl pH 7.4, containing protease inhibitors. The renaturation should be performed over a period of 1.5 hours or more. After renaturation the proteins can be eluted by the addition of 250 mM immidazole. Immidazole is removed by a final dialyzing step against PBS or 50 mM sodium acetate pH 6 buffer plus 200 mM NaCl. The purified protein is stored at 4° C. or frozen at −80° C.

The following alternative method may be used to purify HFLP expressed in E coli when it is present in the form of inclusion bodies. Unless otherwise specified, all of the following steps are conducted at 4-10° C.

Upon completion of the production phase of the E. coli fermentation, the cell culture is cooled to 4-10° C. and the cells are harvested by continuous centrifugation at 15,000 rpm (Heraeus Sepatech). On the basis of the expected yield of protein per unit weight of cell paste and the amount of purified protein required, an appropriate amount of cell paste, by weight, is suspended in a buffer solution containing 100 mM Tris, 50 mM EDTA, pH 7.4. The cells are dispersed to a homogeneous suspension using a high shear mixer.

The cells ware then lysed by passing the solution through a microfluidizer (Microfuidics, Corp. or APV Gaulin, Inc.) twice at 4000-6000 psi. The homogenate is then mixed with NaCl solution to a final concentration of 0.5 M NaCl, followed by centrifugation at 7000×g for 15 min. The resultant pellet is washed again using 0.5M NaCl, 100 mM Tris, 50 mM EDTA, pH 7.4.

The resulting washed inclusion bodies are solubilized with 1.5 M guanidine hydrochloride (GuHCl) for 2-4 hours. After 7000×g centrifugation for 15 min., the pellet is discarded and the HFLP polypeptide-containing supernatant is incubated at 4° C. overnight to allow further GuHCl extraction.

Following high speed centrifugation (30,000×g) to remove insoluble particles, the GuHCl solubilized protein is refolded by quickly mixing the GuHCl extract with 20 volumes of buffer containing 50 mM sodium, pH 4.5, 150 mM NaCl, 2 mM EDTA by vigorous stirring. The refolded diluted protein solution is kept at 4° C. without mixing for 12 hours prior to further purification steps.

To clarify the refolded HFLP polypeptide solution, a previously prepared tangential filtration unit equipped with 0.16 μm membrane filter with appropriate surface area (e.g., Filtron), equilibrated with 40 mM sodium acetate, pH 6.0 is employed. The filtered sample is loaded onto a cation exchange resin (e.g., Poros HS-50, Perseptive Biosystems). The column is washed with 40 mM sodium acetate, pH 6.0 and eluted with 250 mM, 500 mM, 1000 mM, and 1500 mM NaCl in the same buffer, in a stepwise manner. The absorbance at 280 mm of the effluent is continuously monitored. Fractions are collected and further analyzed by SDS-PAGE.

Fractions containing the HFLP polypeptide are then pooled and mixed with 4 volumes of water. The diluted sample is then loaded onto a previously prepared set of tandem columns of strong anion (Poros HQ-50, Perseptive Biosystems) and weak anion (Poros CM-20, Perseptive Biosystems) exchange resins. The columns are equilibrated with 40 mM sodium acetate, pH 6.0. Both columns are washed with 40 mM sodium acetate, pH 6.0, 200 mM NaCl. The CM-20 column is then eluted using a 10 column volume linear gradient ranging from 0.2 M NaCl, 50 mM sodium acetate, pH 6.0 to 1.0 M NaCl, 50 mM sodium acetate, pH 6.5. Fractions are collected under constant A₂₈₀ monitoring of the effluent. Fractions containing the HFLP polypeptide (determined, for instance, by 16% SDS-PAGE) are then pooled.

The resultant HFLP polypeptide exhibits greater than 95% purity after the above refolding and purification steps. No major contaminant bands are observed from Commassie blue stained 16% SDS-PAGE gel when 5 μg of purified protein is loaded. The purified protein is also tested for endotoxin/LPS contamination, and typically the LPS content is less than 0.1 ng/ml according to LAL assays.

### Example 2

### Cloning and Expression of HFLP Protein in a Baculovirus Expression System

In this illustrative example, the plasmid shuttle vector pA2 is used to insert the cloned DNA encoding complete protein, including a secretory signal (leader) sequence derived from the bovine frezzled sequence (Hoang, B., et al., J. Biol. Chem. 271:26131-26137 (1996); GenBank Accession No. U24164), into a baculovirus to express the mature HFLP protein, using standard methods as described by Summers and colleagues (A Manual of Methods for Baculovirus Vectors and Insect Cell Culture Procedures, Texas Agricultural Experimental Station Bulletin No. 1555 (1987)). This expression vector contains the strong polyhedrin promoter of the Autographa californica nuclear polyhedrosis virus (AcMNPV) followed by convenient restriction sites such as Bam HI, Xba I and Asp 718. The polyadenylation site of the simian virus 40 (“SV40”) is used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate a viable virus that express the cloned polynucleotide.

Many other baculovirus vectors could be used in place of the vector above, such as pAc373, pVL941 and pAcIM1, as one skilled in the art would readily appreciate, as long as the construct provides appropriately located signals for transcription, translation, secretion and the like, including a signal peptide and an in-frame AUG as required. Such vectors are described, for instance, by Luckow and coworkers (Virology 170:31-39 (1989)).

The cDNA sequence encoding the full length HFLP protein in the deposited clone, including the AUG initiation codon and the naturally associated leader sequence shown in SEQ ID NO:2, is amplified using PCR oligonucleotide primers corresponding to the 5′ and 3′ sequences of the gene. The 5′ primer has the sequence

5′ GACTGGATCCGCCATCATGG

TTTGCGGATCACGTGGAGGTATGCTACTGCTTCCACGCGTCCGCT

CCATCCTAG 3′ (SEQ ID NO:14) containing the underlined Bam HI restriction enzyme site, an efficient signal for initiation of translation in eukaryotic cells (Kozak, M., J. Mol. Biol. 196:947-950 (1987)), followed by 36 nucleotides encoding the bovine frezzled secretory sequence (Hoang, B., et al., J. Biol. Chem. 271:26131-26137 (1996); GenBank Accession No. U24164), followed by 22 nucleotides of the complete HFLP protein shown in FIGS. 1A and 1B. The 3′ primer has the sequence

5′ GACTGGTACCGGAAGTCGGAAGTCTCCGC 3′ (SEQ ID NO:15) containing the underlined Asp 718 restriction site followed by 19 nucleotides complementary to the 3′ noncoding sequence in FIGS. 1A and 1B.

The amplified fragment is isolated from a 1% agarose gel using a commercially available kit (“Geneclean,” BIO 101 Inc., La Jolla, Calif.). The fragment then is digested with Bam HI and Asp 718 and again is purified on a 1% agarose gel. This fragment is designated herein F1.

The plasmid is digested with the restriction enzymes Bam HI and Asp 718 and optionally, can be dephosphorylated using calf intestinal phosphatase, using routine procedures known in the art. The DNA is then isolated from a 1% agarose gel using a commercially available kit (“Geneclean” BIO 101 Inc., La Jolla, Calif.). This vector DNA is designated herein “V1”.

Fragment F 1 and the dephosphorylated plasmid VI are ligated together with T4 DNA ligase. E. coli HB101 or other suitable E. coli hosts such as XL-1 Blue (Statagene Cloning Systems, La Jolla, Calif.) cells are transformed with the ligation mixture and spread on culture plates. Bacteria are identified that contain the plasmid with the human HFLP gene by digesting DNA from individual colonies using Bam HI and Asp 718 and then analyzing the digestion product by gel electrophoresis. The sequence of the cloned fragment is confirmed by DNA sequencing. This plasmid is designated herein pA2HFLP.

Five μg of the plasmid pA2HFLP is co-transfected with 1.0 μg of a commercially available linearized baculovirus DNA (“BaculoGold™ baculovirus DNA”, Pharmingen, San Diego, Calif.), using the lipofection method described by Felgner and colleaguew colleagues (Proc. Natl. Acad. Sci. USA 84:7413-7417 (1987)). One μg of BaculoGold™ virus DNA and 5 μg of the plasmid pA2HFLP are mixed in a sterile well of a microtiter plate containing 50 μl of serum-free Grace's medium (Life Technologies Inc., Gaithersburg, Md.). Afterwards, 10 μl Lipofectin plus 90 μl Grace's medium are added, mixed and incubated for 15 minutes at room temperature. Then the transfection mixture is added drop-wise to Sf9 insect cells (ATCC CRL 1711) seeded in a 35 mm tissue culture plate with 1 ml Grace's medium without serum. The plate is then incubated for 5 hours at 27° C. The transfection solution is then removed from the plate and 1 ml of Grace's insect medium supplemented with 10% fetal calf serum is added. Cultivation is then continued at 27° C. for four days.

After four days the supernatant is collected and a plaque assay is performed, as described by Summers and Smith (supra). An agarose gel with “Blue Gal” (Life Technologies Inc., Gaithersburg) is used to allow easy identification and isolation of gal-expressing clones, which produce blue-stained plaques. (A detailed description of a “plaque assay” of this type can also be found in the user's guide for insect cell culture and baculovirology distributed by Life Technologies Inc., Gaithersburg, page 9-10). After appropriate incubation, blue stained plaques are picked with the tip of a micropipettor (e.g., Eppendorf). The agar containing the recombinant viruses is then resuspended in a microcentrifuge tube containing 200 μl of Grace's medium and the suspension containing the recombinant baculovirus is used to infect Sf9 cells seeded in 35 mm dishes. Four days later the supernatants of these culture dishes are harvested and then they are stored at 4° C. The recombinant virus is called V-HFLP.

To verify the expression of the HFLP gene Sf9 cells are grown in Grace's medium supplemented with 10% heat-inactivated FBS. The cells are infected with the recombinant baculovirus V-HFLP at a multiplicity of infection (“MOI”) of about 2. If radiolabeled proteins are desired, 6 hours later the medium is removed and is replaced with SF900 II medium minus methionine and cysteine (available from Life Technologies Inc., Rockville, Md.). After 42 hours, 5 μCi of ³⁵S-methionine and 5 μCi ³⁵S-cysteine (available from Amersham) are added. The cells are further incubated for 16 hours and then are harvested by centrifugation. The proteins in the supernatant as well as the intracellular proteins are analyzed by SDS-PAGE followed by autoradiography (if radiolabeled).

Microsequencing of the amino acid sequence of the amino terminus of purified protein may be used to determine the amino terminal sequence of the mature form of the HFLP protein.

### Example 3

### Cloning and Expression of HFLP in Mammalian Cells

A typical mammalian expression vector contains the promoter element, which mediates the initiation of transcription of mRNA, the protein coding sequence, and signals required for the termination of transcription and polyadenylation of the transcript. Additional elements include enhancers, Kozak sequences and intervening sequences flanked by donor and acceptor sites for RNA splicing. Highly efficient transcription can be achieved with the early and late promoters from SV40, the long terminal repeats (LTRs) from Retroviruses, e.g., RSV, HTLVI, HIVI and the early promoter of the cytomegalovirus (CMV). However, cellular elements can also be used (e.g., the human actin promoter). Suitable expression vectors for use in practicing the present invention include, for example, vectors such as pSVL and pMSG (Pharmacia, Uppsala, Sweden), pRSVcat (ATCC 37152), pSV2dhfr (ATCC 37146) and pBC12MI (ATCC 67109). Mammalian host cells that could be used include, human Hela, 293, H9 and Jurkat cells, mouse NIH3T3 and C127 cells, Cos 1, Cos 7 and CV1, quail QC1-3 cells, mouse L cells and Chinese hamster ovary (CHO) cells.

Alternatively, the gene can be expressed in stable cell lines that contain the gene integrated into a chromosome. The co-transfection with a selectable marker such as dhfr, gpt, neomycin, hygromycin allows the identification and isolation of the transfected cells.

The transfected gene can also be amplified to express large amounts of the encoded protein. The DHFR (dihydrofolate reductase) marker is useful to develop cell lines that carry several hundred or even several thousand copies of the gene of interest. Another useful selection marker is the enzyme glutamine synthase (G S; Murphy, et al., Biochem J. 227:277-279 (1991); Bebbington, et al., Bio/Technology 10: 169-175 (1992)). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of proteins.

The expression vectors pC1 and pC4 contain the strong promoter (LTR) of the Rous Sarcoma Virus (Cullen, et al., Mol. Cel. Biol. 5:438-447 (1985)) plus a fragment of the CMV-enhancer (Boshart, et al., Cell 41:521-530 (1985)). Multiple cloning sites, e.g., with the restriction enzyme cleavage sites Bam HI, Xba I and Asp 718, facilitate the cloning of the gene of interest. The vectors contain in addition the 3′ intron, the polyadenylation and termination signal of the rat preproinsulin gene.

### Example 3(a)

### Cloning and Expression in COS CELLS

The expression plasmid, pHFLPHA, is made by cloning a portion of the cDNA encoding the mature form of the HFLP protein into the expression vector pcDNAI/Amp or pcDNAIII (which can be obtained from Invitrogen, Inc.).

The expression vector pcDNAI/amp contains: (1) an E. coli origin of replication effective for propagation in E. coli and other prokaryotic cells; (2) an ampicillin resistance gene for selection of plasmid-containing prokaryotic cells; (3) an SV40 origin of replication for propagation in eukaryotic cells; (4) a CMV promoter, a polylinker, an SV40 intron; (5) several codons encoding a hemagglutinin fragment (i.e., an “HA” tag to facilitate purification) followed by a termination codon and polyadenylation signal arranged so that a cDNA can be conveniently placed under expression control of the CMV promoter and operably linked to the SV40 intron and the polyadenylation signal by means of restriction sites in the polylinker. The HA tag corresponds to an epitope derived from the influenza hemagglutinin protein described by Wilson and colleagues (Cell 37:767 (1984)). The fusion of the HA tag to the target protein allows easy detection and recovery of the recombinant protein with an antibody that recognizes the HA epitope. pcDNAIII contains, in addition, the selectable neomycin marker.

A DNA fragment encoding the complete HFLP polypeptide plus a 12 amino acid secretory sequence derived from the bovine frezzled gene is cloned into the polylinker region of the vector so that recombinant protein expression is directed by the CMV promoter. The plasmid construction strategy is as follows. The HFLP cDNA of the deposited clone is amplified using primers that contain convenient restriction sites, much as described above for construction of vectors for expression of HFLP in E. coli. Suitable primers include the following, which are used in this example. The 5′ primer, containing the underlined Bam HI site, a Kozak sequence, an AUG start codon, 36 nucleotides encoding the secretory signal of the bovine frezzled gene, and 28 nucleotides of the 5′ coding region of the complete HFLP polypeptide, has the following sequence: 5′ GACTGGATCCGCCATCATGGTTTGCGGA

TCACGTGGAGGTATGCTACTGCTTCCACGCGTCCGCTCCATCCTA G 3′ (SEQ ID NO:14). The 3′ primer, containing the underlined Xho I and 20 of nucleotides complementary to the 3′ coding sequence immediately before the stop codon, has the following sequence:

5′ GACTCTCGAGCACTCTTTTCGGGTTTGTTC 3′ (SEQ ID NO:16).

The PCR amplified DNA fragment and the vector, pcDNAI/Amp, are digested with Bam HI and Xho I and then ligated. The ligation mixture is transformed into E. coli strain SURE (Stratagene Cloning Systems, La Jolla, Calif. 92037), and the transformed culture is plated on ampicillin media plates which then are incubated to allow growth of ampicillin resistant colonies. Plasmid DNA is isolated from resistant colonies and examined by restriction analysis or other means for the presence of the fragment encoding the complete HFLP polypeptide

For expression of recombinant HFLP, COS cells are transfected with an expression vector, as described above, using DEAE-dextran, as described, for instance, by Sambrook and coworkers (Molecular Cloning: a Laboratory Manual, Cold Spring Laboratory Press, Cold Spring Harbor, N.Y. (1989)). Cells are incubated under conditions for expression of HFLP by the vector.

Expression of the HFLP-HA fusion protein is detected by radiolabeling and immunoprecipitation, using methods described in, for example Harlow and colleagues (Antibodies: A Laboratory Manual, 2nd Ed.; Cold Spring Harbor Laboratory Press, Cold Spring Harbor, N.Y. (1988)). To this end, two days after transfection, the cells are labeled by incubation in media containing ³⁵S-cysteine for 8 hours. The cells and the media are collected, and the cells are washed and the lysed with detergent-containing RIPA buffer: 150 mM NaCl, 1% NP-40, 0.1% SDS, 1% NP-40, 0.5% DOC, 50 mM TRIS, pH 7.5, as described by Wilson and colleagues (supra). Proteins are precipitated from the cell lysate and from the culture media using an HA-specific monoclonal antibody. The precipitated proteins then are analyzed by SDS-PAGE and autoradiography. An expression product of the expected size is seen in the cell lysate, which is not seen in negative controls.

### Example 3(b)

### Cloning and Expression in CHO Cells

The vector pC4 is used for the expression of HFLP polypeptide. Plasmid pC4 is a derivative of the plasmid pSV2-dhfr (ATCC Accession No. 37146). The plasmid contains the mouse DHFR gene under control of the SV40 early promoter. Chinese hamster ovary- or other cells lacking dihydrofolate activity that are transfected with these plasmids can be selected by growing the cells in a selective medium (alpha minus MEM, Life Technologies) supplemented with the chemotherapeutic agent methotrexate. The amplification of the DHFR genes in cells resistant to methotrexate (MTX) has been well documented (see, e.g., Alt, F. W., et al., J. Biol. Chem. 253:1357-1370 (1978); Hamlin, J. L. and Ma, C. Biochem. et Biophys. Acta, 1097:107-143 (1990); Page, M. J. and Sydenham, M. A. Biotechnology 9:64-68 (1991)). Cells grown in increasing concentrations of MTX develop resistance to the drug by overproducing the target enzyme, DHFR, as a result of amplification of the DHFR gene. If a second gene is linked to the DHFR gene, it is usually co-amplified and over-expressed. It is known in the art that this approach may be used to develop cell lines carrying more than 1,000 copies of the amplified gene(s). Subsequently, when the methotrexate is withdrawn, cell lines are obtained which contain the amplified gene integrated into one or more chromosome(s) of the host cell.

Plasmid pC4 contains for expressing the gene of interest the strong promoter of the long terminal repeat (LTR) of the Rouse Sarcoma Virus (Cullen, et al., Mol. Cell. Biol. 5:438-447 (1985)) plus a fragment isolated from the enhancer of the immediate early gene of human cytomegalovirus (CMV; Boshart, et al., Cell 41:521-530 (1985)). Downstream of the promoter are the following single restriction enzyme cleavage sites that allow the integration of the genes: Bam HI, Xba I, and Asp 718. Behind these cloning sites the plasmid contains the 3′ intron and polyadenylation site of the rat preproinsulin gene. Other high efficiency promoters can also be used for the expression, e.g., the human β-actin promoter, the SV40 early or late promoters or the long terminal repeats from other retroviruses, e.g., HIV and HTLVI. Clontech's Tet-Off and Tet-On gene expression systems and similar systems can be used to express the HFLP polypeptide in a regulated way in mammalian cells (Gossen, M., and Bujard, H. Proc. Natl. Acad. Sci. USA 89:5547-5551 (1992)). For the polyadenylation of the mRNA other signals, e.g., from the human growth hormone or globin genes can be used as well. Stable cell lines carrying a gene of interest integrated into the chromosomes can also be selected upon co-transfection with a selectable marker such as gpt, G418 or hygromycin. It is advantageous to use more than one selectable marker in the beginning, e.g., G418 plus methotrexate.

The plasmid pC4 is digested with the restriction enzymes Bam HI and Asp 718 and then dephosphorylated using calf intestinal phosphates by procedures known in the art. The vector is then isolated from a 1% agarose gel.

The DNA sequence encoding the complete HFLP polypeptide is amplified using PCR oligonucleotide primers corresponding to the 5′ and 3′ sequences of the desired portion of the gene. The 5′ primer containing the underlined Bam HI site, a Kozak sequence, an AUG start codon, 36 nucleotides encoding the secretory leader of the bovine frezzled gene, and 28 nucleotides of the 5′ coding region of the complete HFLP polypeptide, has the following sequence:

5′ GACTGGATCCGCCATCATGGTTTGCGGATCACGTGGAGGTATGCT ACTGCTTCCACGCGTCCGCTCCATCCTAG 3′ (SEQ ID NO:14). The 3′ primer, containing the underlined Asp 718 restriction site and 19 of nucleotides complementary to the 3′ noncoding sequence immediately before the stop codon as shown in FIGS. 1A and 1B (SEQ ID NO:1), has the following sequence: 5′ GAC TGG TAC CGG AAG TCG GAA GTC TCC GC 3′ (SEQ ID NO:15).

The amplified fragment is digested with the endonucleases Bam HI and Asp 718 and then purified again on a 1% agarose gel. The isolated fragment and the dephosphorylated vector are then ligated with T4 DNA ligase. E. coli HB101 or XL-1 Blue cells are then transformed and bacteria are identified that contain the fragment inserted into plasmid pC4 using, for instance, restriction enzyme analysis.

Chinese hamster ovary cells lacking an active DHFR gene are used for transfection. Five μg of the expression plasmid pC4 is cotransfected with 0.5 μg of the plasmid pSVneo using lipofectin (Felgner, et al., supra). The plasmid pSV2-neo contains a dominant selectable marker, the neo gene from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) in alpha minus MEM supplemented with 10, 25, or 50 ng/ml of metothrexate plus 1 mg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 μM, 2 μM, 5 μM, 10 mM, 20 mM). The same procedure is repeated until clones are obtained which grow at a concentration of 100-200 μM. Expression of the desired gene product is analyzed, for instance, by SDS-PAGE and Western blot or by reversed phase HPLC analysis.

### Example 4

### Tissue Distribution of HFLP mRNA Expression

Northern blot analysis is carried out to examine HFLP gene expression in human tissues, using methods described by, among others, Sambrook and colleagues (supra). A cDNA probe containing the entire nucleotide sequence of the HFLP protein (SEQ ID NO:1) is labeled with ³²P using the rediprime™ DNA labeling system (Amersham Life Science), according to manufacturer's instructions. After labeling, the probe is purified using a CHROMA SPIN-100™ column (Clontech Laboratories, Inc.), according to manufacturer's protocol number PT1200-1. The purified labeled probe is then used to examine various human tissues for HFLP mRNA.

Multiple Tissue Northern (MTN) blots containing various human tissues (H) or human immune system tissues (IM) are obtained from Clontech and are examined with the labeled probe using ExpressHyb™ hybridization solution (Clontech) according to manufacturer's protocol number PT1190-1. Following hybridization and washing, the blots are mounted and exposed to film at −70° C. overnight, and films developed according to standard procedures.

### Example 5

Protein Fusions of HFLP

HFLP polypeptides are preferably fused to other proteins. These fusion proteins can be used for a variety of applications. For example, fusion of HFLP polypeptides to His-tag, HA-tag, protein A, IgG domains, and maltose binding protein facilitates purification (see EP A 394,827; Traunecker, et al., Nature 331:84-86 (1988)). Similarly, fusion to IgG-1, IgG-3, and albumin increases the halflife time in vivo. Nuclear localization signals fused to HFLP polypeptides can target the protein to a specific subcellular localization, while covalent heterodimer or homodimers can increase or decrease the activity of a fusion protein. Fusion proteins can also create chimeric molecules having more than one function. Finally, fusion proteins can increase solubility and/or stability of the fused protein compared to the non-fused protein. All of the types of fusion proteins described above can be made by modifying the following protocol, which outlines the fusion of a polypeptide to an IgG molecule.

Briefly, the human Fc portion of the IgG molecule can be PCR amplified, using primers that span the 5′ and 3′ ends of the sequence described below. These primers also should have convenient restriction enzyme sites that will facilitate cloning into an expression vector, preferably a mammalian expression vector.

For example, if pC4 (ATCC Accession No. 209646) is used, the human Fc portion can be ligated into the Bam HI cloning site. Note that the 3′ Bam HI site should be destroyed. Next, the vector containing the human Fc portion is re-restricted with Bam HI, linearizing the vector, and HFLP polynucleotide, isolated, for instance, by the PCR protocol described in Example 1, is ligated into this Bam HI site. Note that the polynucleotide is cloned without a stop codon, otherwise a fusion protein will not be produced.

If the naturally occurring signal sequence is used to produce the secreted protein, pC4 does not need a second signal peptide. Alternatively, if the naturally occurring signal sequence is not used, the vector can be modified to include a heterologous signal sequence (see, e.g., WO 96/34891).

The human IgG Fc region is shown below as SEQ ID NO:17:

GGGATCCGGAGCCCAAATCTTCTGACAAAACTCACACATGCCCACCGTGC
CCAGCACCTGAATTCGAGGGTGCACCGTCAGTCTTCCTCTTCCCCCCAAA
ACCCAAGGACACCCTCATGATCTCCCGGACTCCTGAGGTCACATGCGTGG
TGGTGGACGTAAGCCACGAAGACCCTGAGGTCAAGTTCAACTGGTACGTG
GACGAGCACGTACCGTGTGGTCAGCGTCCTCACCGTCCTGCACCAGGACT
GGCTGAATGGCAAGGAGTACAAGTGCAAGGTCTCCAACAAAGCCCTCCCA
ACCCCCATCGAGAAAACCATCTCCAAAGCCAAAGGGCAGCCCCGAGAACC
ACAGGTGTACACCCTGCCCCATCCCGGGATGAGCTGACCAAGAACCAGGT
CAGCCTGACCTGCCTGGTCAAAGGCTTCTATCCAAGCGACATCGCCGTGG
AGTGGGAGAGCAATGGGCAGCCGGAGAACAACTACAAGACCACGCCTCCC
GTGCTGGACTCCGACGGCTCCTTCTTCCTCTACAGCAAGCTCACCGTGGA
CAAGAGCAGGTGGCAGCAGGGGAACGTCTTCTCATGCTCCGTGATGCATG
AGGCTCTGCACAACCACTACACGCAGAAGAGCCTCTCCCTGTCTCCGGGT
AAATGAGTGCGACGGCCGCGACTCTAGAGGAT

### Example 6

### Production of an Antibody

The antibodies of the present invention can be prepared by a variety of methods (see, Current Protocols, Chapter 2). For example, cells expressing HFLP are administered to an animal to induce the production of sera containing polyclonal antibodies. In a preferred method, a preparation of HFLP protein is prepared and purified to render it substantially free of natural contaminants. Such a preparation is then introduced into an animal in order to produce polyclonal antisera of greater specific activity.

In the most preferred method, the antibodies of the present invention are monoclonal antibodies (or protein binding fragments thereof). Such monoclonal antibodies can be prepared using hybridoma technology (Kohler, et al., Nature 256:495 (1975); Kohler, et al., Eur. J. Immunol. 6:511 (1976); Kohler, et al., Eur. J. Immunol. 6:292 (1976); Hammerling, et al., in: Monoclonal Antibodies and T-Cell Hybridomas, Elsevier, N.Y., pp. 563-681 (1981)). In general, such procedures involve immunizing an animal (preferably a mouse) with HFLP polypeptide or, more preferably, with a secreted HFLP polypeptide-expressing cell. Such cells may be cultured in any suitable tissue culture medium; however, it is preferable to culture cells in Earle's modified Eagle's medium supplemented with 10% fetal bovine serum (inactivated at about 56° C.), and supplemented with about 10 g/l of nonessential amino acids, about 1,000 U/ml of penicillin, and about 100 μg/ml of streptomycin.

The splenocytes of such mice are extracted and fused with a suitable myeloma cell line. Any suitable myeloma cell line may be employed in accordance with the present invention; however, it is preferable to employ the parent myeloma cell line (SP2O), available from the ATCC. After fusion, the resulting hybridoma cells are selectively maintained in HAT medium, and then cloned by limiting dilution as described by Wands and coworkers (Gastroenterology 80:225-232 (1981)). The hybridoma cells obtained through such a selection are then assayed to identify clones which secrete antibodies capable of binding the HFLP polypeptide.

Alternatively, additional antibodies capable of binding to HFLP polypeptide can be produced in a two-step procedure using anti-idiotypic antibodies. Such a method makes use of the fact that antibodies are themselves antigens, and therefore, it is possible to obtain an antibody which binds to a second antibody. In accordance with this method, protein specific antibodies are used to immunize an animal, preferably a mouse. The splenocytes of such an animal are then used to produce hybridoma cells, and the hybridoma cells are screened to identify clones which produce an antibody whose ability to bind to the HFLP protein-specific antibody can be blocked by HFLP. Such antibodies comprise anti-idiotypic antibodies to the HFLP protein-specific antibody and can be used to immunize an animal to induce formation of further HFLP protein-specific antibodies.

It will be appreciated that Fab and F(ab′)2 and other fragments of the antibodies of the present invention may be used according to the methods disclosed herein. Such fragments are typically produced by proteolytic cleavage, using enzymes such as papain (to produce Fab fragments) or pepsin (to produce F(ab′)2 fragments). Alternatively, secreted HFLP protein-binding fragments can be produced through the application of recombinant DNA technology or through synthetic chemistry.

For in vivo use of antibodies in humans, it may be preferable to use “humanized” chimeric monoclonal antibodies. Such antibodies can be produced using genetic constructs derived from hybridoma cells producing the monoclonal antibodies described above. Methods for producing chimeric antibodies are known in the art (see, for review, Morrison, Science 229:1202 (1985); Oi, et al., BioTechniques 4:214 (1986); Cabilly, et al., U.S. Pat. No. 4,816,567; Taniguchi, et al., EP 171496; Morrison, et al., EP 173494; Neuberger, et al., WO 8601533; Robinson, et al., WO 8702671; Boulianne, et al., Nature 312:643 (1984); Neuberger, et al., Nature 314:268 (1985)).

### Example 7

### Production Of HFLP Protein For High-Throughput Screening Assays

The following protocol produces a supernatant containing HFLP polypeptide to be tested. This supernatant can then be used in the Screening Assays described in Examples 9-16.

First, dilute Poly-D-Lysine (644 587 Boehringer-Mannheim) stock solution (1 mg/ml in PBS) 1:20 in PBS (without calcium or magnesium 17-516F Biowhittaker) for a working solution of 50 μg/ml. Add 200 ul of this solution to each well (24 well plates) and incubate at RT for 20 minutes. Be sure to distribute the solution over each well (note: a 12-channel pipetter may be used with tips on every other channel). Aspirate the Poly-D-Lysine solution and rinse with 1 ml PBS (Phosphate Buffered Saline). The PBS should remain in the well until just prior to plating the cells and plates may be poly-lysine coated in advance for up to two weeks.

Plate 293T cells (do not carry cells past P+20) at 2×10⁵ cells/well in 0.5 ml DMEM (Dulbecco's Modified Eagle Medium)(with 4.5 G/L glucose and L-glutamine (12-604F Biowhittaker))/10% heat inactivated FBS(14-503F Biowhittaker)/1× Penstrep (17-602E Biowhittaker). Let the cells grow overnight.

The next day, mix together in a sterile solution basin: 300 μl Lipofectamine (18324-012 Gibco/BRL) and 5 ml Optimem 1 (31985070 Gibco/BRL)/96-well plate. With a small volume multi-channel pipetter, aliquot approximately 2 μg of an expression vector containing a polynucleotide insert, produced by the methods described in Example 2 or 3, into an appropriately labeled 96-well round bottom plate. With a multi-channel pipetter, add 50 μl of the Lipofectamine/Optimem I mixture to each well. Pipette up and down gently to mix. Incubate at room temperature (RT) for 15-45 minutes. After about 20 minutes, use a multi-channel pipetter to add 150 μl Optimem I to each well. As a control, one plate of vector DNA lacking an insert should be transfected with each set of transfections.

Preferably, the transfection should be performed by tag-teaming the following tasks. By tag-teaming, hands on time is cut in half, and the cells do not spend too much time on PBS. First, person A aspirates the media from four 24-well plates of cells, and then person B rinses each well with 0.5-1 ml PBS. Person A then aspirates the PBS rinse, and person B, using a 12-channel pipetter with tips on every other channel, adds the 200 μl of DNA/Lipofectamine/Optimem I complex to the odd wells first, then to the even wells, to each row on the 24-well plates. Incubate at 37° C. for 6 hours.

While cells are incubating, prepare appropriate media, either 1% BSA in DMEM with 1× penstrep, or HGS CHO-5 media (116.6 mg/L of CaCl₂ (anhyd); 0.00130 mg/L CuSO₄.5H₂O; 0.050 mg/L of Fe(NO₃)₃-9H₂O; 0.417 mg/L of FeSO₄-7H₂O; 311.80 mg/L of Kcl; 28.64 mg/L of MgCl₂; 48.84 mg/L of MgSO₄; 6995.50 mg/L of NaCl; 2400.0 mg/L of NaHCO₃; 62.50 mg/L of NaH₂PO₄—H₂O; 71.02 mg/L of Na₂HPO₄; 0.4320 mg/L of ZnSO₄-7H₂O; 0.002 mg/L of Arachidonic Acid; 1.022 mg/L of Cholesterol; 0.070 mg/L of DL-alpha-Tocopherol-Acetate; 0.0520 mg/L of Linoleic Acid; 0.010 mg/L of Linolenic Acid; 0.010 mg/L of Myristic Acid; 0.010 mg/L of Oleic Acid; 0.010 mg/L of Palmitric Acid; 0.010 mg/L of Palmitic Acid; 100 mg/L of Pluronic F-68; 0.010 mg/L of Stearic Acid; 2.20 mg/L of Tween 80; 4551 mg/L of D-Glucose; 130.85 mg/ml of L-Alanine; 147.50 mg/ml of L-Arginine-HCL; 7.50 mg/ml of L-Asparagine-H₂O; 6.65 mg/ml of L-Aspartic Acid; 29.56 mg/ml of L-Cystine-2HCL-H₂O; 31.29 mg/ml of L-Cystine-2HCL; 7.35 mg/ml of L-Glutamic Acid; 365.0 mg/ml of L-Glutamine; 18.75 mg/ml of Glycine; 52.48 mg/ml of L-Histidine-HCL-H₂O; 106.97 mg/ml of L-Isoleucine; 111.45 mg/ml of L-Leucine; 163.75 mg/ml of L-Lysine HCL; 32.34 mg/ml of L-Methionine; 68.48 mg/ml of L-Phenylalainine; 40.0 mg/ml of L-Proline; 26.25 mg/ml of L-Serine; 101.05 mg/ml of L-Threonine; 19.22 mg/ml of L-Tryptophan; 91.79 mg/ml of L-Tryrosine-2Na-2H₂O; and 99.65 mg/ml of L-Valine; 0.0035 mg/L of Biotin; 3.24 mg/L of D-Ca Pantothenate; 11.78 mg/L of Choline Chloride; 4.65 mg/L of Folic Acid; 15.60 mg/L of i-Inositol; 3.02 mg/L of Niacinamide; 3.00 mg/L of Pyridoxal HCL; 0.031 mg/L of Pyridoxine HCL; 0.319 mg/L of Riboflavin; 3.17 mg/L of Thiamine HCL; 0.365 mg/L of Thymidine; 0.680 mg/L of Vitamin B₁₂; 25 mM of HEPES Buffer; 2.39 mg/L of Na Hypoxanthine; 0.105 mg/L of Lipoic Acid; 0.081 mg/L of Sodium Putrescine-2HCL; 55.0 mg/L of Sodium Pyruvate; 0.0067 mg/L of Sodium Selenite; 20 uM of Ethanolamine; 0.122 mg/L of Ferric Citrate; 41.70 mg/L of Methyl-B-Cyclodextrin complexed with Linoleic Acid; 33.33 mg/L of Methyl-B-Cyclodextrin complexed with Oleic Acid; 10 mg/L of Methyl-B-Cyclodextrin complexed with Retinal Acetate. Adjust osmolarity to 327 mOsm) with 2 mm glutamine and 1×penstrep. (BSA (81-068-3 Bayer) 100 gm dissolved in 1L DMEM for a 10% BSA stock solution). Filter the media and collect 50 ul for endotoxin assay in 15 ml polystyrene conical.

The transfection reaction is terminated, preferably by tag-teaming, at the end of the incubation period. Person A aspirates the transfection media, while person B adds 1.5 ml appropriate media to each well. Incubate at 37° C. for 45 or 72 hours depending on the media used: 1% BSA for 45 hours or CHO-5 for 72 hours.

On day four, using a 300 μl multichannel pipetter, aliquot 600 μl in one 1 ml deep well plate and the remaining supernatant into a 2 ml deep well. The supernatants from each well can then be used in the assays described in Examples 9 to 16.

It is specifically understood that when activity is obtained in any of the assays described below using a supernatant, the activity originates from either the HFLP polypeptide directly (e.g., as a secreted protein) or by HFLP-inducing expression of other proteins, which are then secreted into the supernatant. Thus, the invention further provides a method of identifying the protein in the supernatant characterized by an activity in a particular assay.

### Example 8

### Construction of GAS Reporter Construct

One signal transduction pathway involved in the differentiation and proliferation of cells is called the Jaks-STATs pathway. Activated proteins in the Jaks-STATs pathway bind to gamma activation site “GAS” elements or interferon-sensitive responsive element (“ISRE”), located in the promoter of many genes. The binding of a protein to these elements alter the expression of the associated gene.

GAS and ISRE elements are recognized by a class of transcription factors called Signal Transducers and Activators of Transcription, or “STATs.” There are six members of the STATs family. Stat1 and Stat3 are present in many cell types, as is Stat2 (as response to IFN-alpha is widespread). Stat4 is more restricted and is not in many cell types though it has been found in T helper class I, cells after treatment with IL-12. Stat5 was originally called mammary growth factor, but has been found at higher concentrations in other cells including myeloid cells. It can be activated in tissue culture cells by many cytokines.

The STATs are activated to translocate from the cytoplasm to the nucleus upon tyrosine phosphorylation by a set of kinases known as the Janus Kinase (“Jaks”) family. Jaks represent a distinct family of soluble tyrosine kinases and include Tyk2, Jak1, Jak2, and Jak3. These kinases display significant sequence similarity and are generally catalytically inactive in resting cells.

The Jaks are activated by a wide range of receptors summarized in the Table below (adapted from review by Schidler and Darnell, Ann. Rev. Biochem. 64:621-51 (1995)). A cytokine receptor family, capable of activating Jaks, is divided into two groups: (a) Class 1 includes receptors for IL-2, IL-3, IL-4, IL-6, IL-7, IL-9, IL-11, IL-12, IL-15, Epo, PRL, GH, G-CSF, GM-CSF, LIF, CNTF, and thrombopoietin; and (b) Class 2 includes IFN-a, IFN-g, and IL-10. The Class 1 receptors share a conserved cysteine motif (a set of four conserved cysteines and one tryptophan) and a WSXWS motif (a membrane proxial region encoding Trp-Ser-Xxx-Trp-Ser (SEQ ID NO:18)).

Thus, on binding of a ligand to a receptor, Jaks are activated, which in turn activate STATs, which then translocate and bind to GAS elements. This entire process is encompassed in the Jaks-STATs signal transduction pathway.

Therefore, activation of the Jaks-STATs pathway, reflected by the binding of the GAS or the ISRE element, can be used to indicate proteins involved in the proliferation and differentiation of cells. For example, growth factors and cytokines are known to activate the Jaks-STATs pathway (see table below). Thus, by using GAS elements linked to reporter molecules, activators of the Jaks-STATs pathway can be identified.

| | | JAKs | | | | |
|---|---|---|---|---|---|---|
| Ligand | tyk2 | Jak1 | Jak2 | Jak3 | STATS | GAS(elements) or ISRE |
| IFN family | | | | | | |
| IFN-a/b | + | + | − | − | 1, 2, 3 | ISRE |
| IFN-g | | + | + | − | 1 | GAS (IRF1 > Lys6 > IFP) |
| Il-10 | + | ? | ? | − | 1, 3 | |
| gp130 family | | | | | | |
| IL-6 (Pleiotrohic) | + | + | + | ? | 1, 3 | GAS (IRF1 > Lys6 > IFP) |
| Il-11(Pleiotrohic) | ? | + | ? | ? | 1, 3 | |
| OnM(Pleiotrohic) | ? | + | + | ? | 1, 3 | |
| LIF(Pleiotrohic) | ? | + | + | ? | 1, 3 | |
| CNTF(Pleiotrohic) | −/+ | + | + | ? | 1, 3 | |
| G-CSF(Pleiotrohic) | ? | + | ? | ? | 1, 3 | |
| IL-12(Pleiotrohic) | + | − | + | + | 1, 3 | |
| g-C family | | | | | | |
| IL-2 (lymphocytes) | − | + | − | + | 1, 3, 5 | GAS |
| IL-4 (lymph/myeloid) | − | + | − | + | 6 | GAS (IRF1 = IFP >> Ly6)(IgH) |
| IL-7 (lymphocytes) | − | + | − | + | 5 | GAS |
| IL-9 (lymphocytes) | − | + | − | + | 5 | GAS |
| IL-13 (lymphocyte) | − | + | ? | ? | 6 | GAS |
| IL-15 | ? | + | ? | + | 5 | GAS |
| gp140 family | | | | | | |
| IL-3 (myeloid) | − | − | + | − | 5 | GAS (IRF1 > IFP >> Ly6) |
| IL-5 (myeloid) | − | − | + | − | 5 | GAS |
| GM-CSF (myeloid) | − | − | + | − | 5 | GAS |
| Growth hormone family | | | | | | |
| GH | ? | − | + | − | 5 | |
| PRL | ? | +/− | + | − | 1, 3, 5 | |
| EPO | ? | − | + | − | 5 | GAS(B-GAS > IRF1 = |
| | | | | | | IFP >> Ly6) |
| Receptor Tyrosine Kinases | | | | | | |
| EGF | ? | + | + | − | 1, 3 | GAS (IRF1) |
| PDGF | ? | + | + | − | 1, 3 | |
| CSF-1 | ? | + | + | − | 1, 3 | GAS (not IRF1) |

To construct a synthetic GAS containing promoter element, which is used in the Biological Assays described in Examples 9 and 10, a PCR based strategy is employed to generate a GAS-SV40 promoter sequence. The 5′ primer contains four tandem copies of the GAS binding site found in the IRF1 promoter and previously demonstrated to bind STATs upon induction with a range of cytokines (Rothman, et al., Immunity 1:457-468 (1994)), although other GAS or ISRE elements can be used instead. The 5′ primer also contains 18 bp of sequence complementary to the SV40 early promoter sequence and is flanked with an Xho I site. The sequence of the 5′ primer is: 5′-GCG CCT CGA GAT TTC CCC GAA ATC TAG ATT TCC CCG AAA TGA TTT CCC CGA AAT GAT TTC CCC GAA ATA TCT GCC ATC TCA ATT AG-3′ (SEQ ID NO:19).

The downstream primer is complementary to the SV40 promoter and is flanked with a Hin dIII site: 5′-GCGGCAAGCTTTTTGCAAAGCCTAGGC-3′ (SEQ ID NO:20).

PCR amplification is performed using the SV40 promoter template present in the b-gal:promoter plasmid obtained from Clontech. The resulting PCR fragment is digested with Xho I and Hin dIII and subcloned into BLSK2-(Stratagene). Sequencing with forward and reverse primers confirms that the insert contains the following sequence: 5′-CTC GAG ATT TCC CCG AAA TCT AGA TTT CCC CGA AAT GAT TTC CCC GAA ATG ATT TCC CCG AAA TAT CTG CCA TCT CAA TTA GTC AGC AAC CAT AGT CCC GCC CCT AAC TCC GCC CAT CCC GCC CCT AAC TCC GCC CAG TTC CGC CCA TTC TCC GCC CCA TGG CTG ACT AAT TTT TTT TAT TTA TGC AGA GGC CGA GGC CGC CTC GGC CTC TGA GCT ATT CCA GAA GTA GTG AGG AGG CTT TTT TGG AGG CCT AGG CTT TTG CAA AAA GCT T-3′ (SEQ ID NO:21).

With this GAS promoter element linked to the SV40 promoter, a GAS:SEAP2 reporter construct is next engineered. Here, the reporter molecule is a secreted alkaline phosphatase (SEAP). Clearly, however, any reporter molecule can be instead of SEAP, in this or in any of the other Examples. Well known reporter molecules that can be used instead of SEAP include chloramphenicol acetyltransferase (CAT), luciferase, alkaline phosphatase, B-galactosidase, green fluorescent protein (GFP), or any protein detectable by an antibody.

The above sequence confirmed synthetic GAS-SV40 promoter element is subcloned into the pSEAP-Promoter vector obtained from Clontech using Hin dIII and Xho I, effectively replacing the SV40 promoter with the amplified GAS: SV40 promoter element, to create the GAS-SEAP vector. However, this vector does not contain a neomycin resistance gene, and therefore, is not preferred for mammalian expression systems.

Thus, in order to generate mammalian stable cell lines expressing the GAS-SEAP reporter, the GAS-SEAP cassette is removed from the GAS-SEAP vector using Sal I and Not I, and inserted into a backbone vector containing the neomycin resistance gene, such as pGFP-1 (Clontech), using these restriction sites in the multiple cloning site, to create the GAS-SEAP/Neo vector. Once this vector is transfected into mammalian cells, this vector can then be used as a reporter molecule for GAS binding as described in Examples 9 and 10.

Other constructs can be made using the above description and replacing GAS with a different promoter sequence. For example, construction of reporter molecules containing NF-κB and EGR promoter sequences are described in Examples 11 and 12. However, many other promoters can be substituted using the protocols described in these Examples. For instance, SRE, IL-2, NFAT, or Osteocalcin promoters can be substituted, alone or in combination (e.g., GAS/NF-κB/EGR, GAS/NF-κB, 11-2/NFAT, or NF-κB/GAS). Similarly, other cell lines can be used to test reporter construct activity, such as HeLa (epithelial), HUVEC (endothelial), Reh (B-cell), Saos-2 (osteoblast), HUVAC (aortic), or Cardiomyocyte.

### Example 9

### High-Throughput Screening Assay for T-Cell Activity

The following protocol is used to assess T-cell activity of HFLP by determining whether HFLP supernatant proliferates and/or differentiates T-cells. T-cell activity is assessed using the GAS/SEAP/Neo construct produced, for example, in Example 8. Thus, factors that increase SEAP activity indicate the ability to activate the Jaks-STATs signal transduction pathway. The T-cell used in this assay is Jurkat T-cells (ATCC Accession No. TIB-152), although Molt-3 cells (ATCC Accession No. CRL-1552) and Molt-4 cells (ATCC Accession No. CRL-1582) cells can also be used.

Jurkat T-cells are lymphoblastic CD4+ Th1 helper cells. In order to generate stable cell lines, approximately 2 million Jurkat cells are transfected with the GAS-SEAP/neo vector using DMRIE-C (Life Technologies; transfection procedure described below). The transfected cells are seeded to a density of approximately 20,000 cells per well and transfectants resistant to 1 mg/ml genticin selected. Resistant colonies are expanded and then tested for their response to increasing concentrations of interferon gamma. The dose response of a selected clone is demonstrated.

Specifically, the following protocol will yield sufficient cells for 75 wells containing 200 μl of cells. Thus, it is either scaled up, or performed in multiple to generate sufficient cells for multiple 96 well plates. Jurkat cells are maintained in RPMI+10% serum with 1% Pen-Strep. Combine 2.5 mls of OPTI-MEM (Life Technologies) with 10 μg of plasmid DNA in a T25 flask. Add 2.5 ml OPTI-MEM containing 50 μl of DMRIE-C and incubate at room temperature for 15-45 min.

During the incubation period, count cell concentration, spin down the required number of cells (10⁷ per transfection), and resuspend in OPTI-MEM to a final concentration of 10⁷ cells/ml. Then add 1 ml of 1×10⁷ cells in OPTI-MEM to T25 flask and incubate at 37° C. for 6 hrs. After the incubation, add 10 ml of RPMI+15% serum.

The Jurkat:GAS-SEAP stable reporter lines are maintained in RPMI+10% serum, 1 mg/ml Genticin, and 1% Pen-Strep. These cells are treated with supernatants containing HFLP polypeptides or HFLP-induced polypeptides as produced by the protocol described in Example 7.

On the day of treatment with the supernatant, the cells should be washed and resuspended in fresh RPMI+10% serum to a density of 500,000 cells per ml. The exact number of cells required will depend on the number of supernatants being screened. For one 96 well plate, approximately 10 million cells (for 10 plates, 100 million cells) are required.

Transfer the cells to a triangular reservoir boat, in order to dispense the cells into a 96 well dish, using a 12 channel pipette. Using a 12 channel pipette, transfer 200 μl of cells into each well (therefore adding 100,000 cells per well).

After all the plates have been seeded, 50 μl of the supernatants are transferred directly from the 96 well plate containing the supernatants into each well using a 12 channel pipette. In addition, a dose of exogenous interferon gamma (0.1, 1.0, 10 ng) is added to wells H9, H10, and H11 to serve as additional positive controls for the assay.

The 96 well dishes containing Jurkat cells treated with supernatants are placed in an incubator for 48 hrs (note: this time is variable between 48-72 hrs). 35 μl samples from each well are then transferred to an opaque 96 well plate using a 12 channel pipette. The opaque plates should be covered (using sellophene covers) and stored at −20° C. until SEAP assays are performed according to Example 13. The plates containing the remaining treated cells are placed at 4° C. and serve as a source of material for repeating the assay on a specific well if desired.

As a positive control, 100 Unit/ml interferon gamma can be used which is known to activate Jurkat T cells. Over 30 fold induction is typically observed in the positive control wells.

### Example 10

### High-Throughput Screening Assay Identifying Myeloid Activity

The following protocol is used to assess myeloid activity of HFLP by determining whether HFLP proliferates and/or differentiates myeloid cells. Myeloid cell activity is assessed using the GAS/SEAP/Neo construct produced in Example 8. Thus, factors that increase SEAP activity indicate the ability to activate the Jaks-STATS signal transduction pathway. The myeloid cell used in this assay is U937, a pre-monocyte cell line, although TF-1, HL60, or KG1 can be used.

To transiently transfect U937 cells with the GAS/SEAP/Neo construct produced in Example 8, a DEAE-Dextran method (Kharbanda, et. al., Cell Growth & Differentiation 5:259-265 (1994)) is used. First, harvest 2×10⁷ U937 cells and wash with PBS. The U937 cells are usually grown in RPMI 1640 medium containing 10% heat-inactivated fetal bovine serum (FB S) supplemented with 100 units/ml penicillin and 100 mg/ml streptomycin.

Next, suspend the cells in 1 ml of 20 mM Tris-HCl (pH 7.4) buffer containing 0.5 mg/ml DEAE-Dextran, 8 μg GAS-SEAP2 plasmid DNA, 140 mM NaCl, 5 mM KCl, 375 μM Na₂HPO₄₀.7H₂₀, 1 mM MgCl₂, and 675 μM CaCl₂. Incubate at 37° C. for 45 min.

Wash the cells with RPMI 1640 medium containing 10% FBS and then resuspend in 10 ml complete medium and incubate at 37° C. for 36 hr.

The GAS-SEAP/U937 stable cells are obtained by growing the cells in 400 μg/ml G418. The G418-free medium is used for routine growth but every one to two months, the cells should be re-grown in 400 μg/ml G418 for couple of passages.

These cells are tested by harvesting 1×10⁸ cells (this is enough for ten 96-well plates assay) and wash with PBS. Suspend the cells in 200 ml above described growth medium, with a final density of 5×10⁵ cells/ml. Plate 200 μl cells per well in the 96-well plate (or 1×10⁵ cells/well).

Add 50 μl of the supernatant prepared by the protocol described in Example 7. Incubate at 37° C. for 48 to 72 hr. As a positive control, 100 Unit/ml interferon gamma can be used which is known to activate U937 cells. Over 30 fold induction is typically observed in the positive control wells. SEAP assay the supernatant according to the protocol described in Example 13.

### Example 11

### High-Throughput Screening Assay Identifying Neuronal Activity

When cells undergo differentiation and proliferation, a group of genes are activated through many different signal transduction pathways. One of these genes, EGR1 (early growth response gene 1), is induced in various tissues and cell types upon activation. The promoter of EGR1 is responsible for such induction. Using the EGR1 promoter linked to reporter molecules, activation of cells can be assessed by HFLP.

Particularly, the following protocol is used to assess neuronal activity in PC12 cell lines. PC12 cells (rat phenochromocytoma cells) are known to proliferate and/or differentiate by activation with a number of mitogens, such as TPA (tetradecanoyl phorbol acetate), NGF (nerve growth factor), and EGF (epidermal growth factor). The EGR1 gene expression is activated during this treatment. Thus, by stably transfecting PC 12 cells with a construct containing an EGR promoter linked to SEAP reporter, activation of PC12 cells by HFLP can be assessed.

The EGR/SEAP reporter construct can be assembled by the following protocol. The EGR-1 promoter sequence (nucleotides −633 to +1; Sakamoto, K., et al., Oncogene 6:867-871 (1991)) can be PCR amplified from human genomic DNA using the 5′ primer 5′-GCG CTC GAG GGA TGA CAG CGA TAG AAC CCC GG-3′ (SEQ ID NO:22) and the 3′ primer 5′-GCG AAG CTT CGC GAC TCC CCG GAT CCG CCT C-3′ (SEQ ID NO:23).

Using the GAS:SEAP/Neo vector produced in Example 8, EGR1 amplified product can then be inserted into this vector. Linearize the GAS:SEAP/Neo vector using restriction enzymes Xho I and Hin dEi, removing the GAS/SV40 stuffer. Restrict the EGR1 amplified product with these same enzymes. Ligate the vector and the EGR1 promoter.

To prepare 96 well-plates for cell culture, two mls of a coating solution (1:30 dilution of collagen type I (Upstate Biotech Inc. Cat#08-115) in 30% ethanol (filter sterilized)) is added per one 10 cm plate or 50 ml per well of the 96-well plate, and allowed to air dry for 2 hr.

PC12 cells are routinely grown in RPMI-1640 medium (Bio Whittaker) containing 10% horse serum (JRH BIOSCIENCES, Cat. # 12449-78P), 5% heat-inactivated fetal bovine serum (FBS) supplemented with 100 units/ml penicillin and 100 μg/ml streptomycin on a precoated 10 cm tissue culture dish. One to four split is done every three to four days. Cells are removed from the plates by scraping and resuspended with pipetting up and down for more than 15 times.

Transfect the EGR/SEAP/Neo construct into PC12 using the Lipofectamine protocol described in Example 7. EGR-SEAP/PC12 stable cells are obtained by growing the cells in 300 μg/ml G418. The G418-free medium is used for routine growth but every one to two months, the cells should be re-grown in 300 μg/ml G418 for couple of passages.

To assay for neuronal activity, a 10 cm plate with cells around 70 to 80% confluent is screened by removing the old medium. Wash the cells once with PBS (Phosphate buffered saline). Then starve the cells in low serum medium (RPMI-1640 containing 1% horse serum and 0.5% FBS with antibiotics) overnight.

The next morning, remove the medium and wash the cells with PBS. Scrape off the cells from the plate, suspend the cells well in 2 ml low serum medium. Count the cell number and add more low serum medium to reach final cell density as 5×10⁵ cells/ml.

Add 200 μl of the cell suspension to each well of 96-well plate (equivalent to 1×10⁵ cells/well). Add 50 μl supernatant produced by Example 11, 37° C. for 48 to 72 hr. As a positive control, a growth factor known to activate PC12 cells through EGR can be used, such as 50 ng/μl of Neuronal Growth Factor (NGF). Over fifty-fold induction of SEAP is typically seen in the positive control wells. SEAP assay the supernatant according to Example 13.

### Example 12

### High-Throughput Screening Assay for T-cell Activity

NF-kB (Nuclear Factor-kB) is a transcription factor activated by a wide variety of agents including the inflammatory cytokines 1L-1 and TNF, CD30 and CD40, lymphotoxin-a and lymphotoxin-b, by exposure to LPS or thrombin, and by expression of certain viral gene products. As a transcription factor, NF-kB regulates the expression of genes involved in immune cell activation, control of apoptosis (NF-kB appears to shield cells from apoptosis), B- and T-cell development, anti-viral and antimicrobial responses, and multiple stress responses.

In non-stimulated conditions, NF-κB is retained in the cytoplasm with I-kB (Inhibitor kB). However, upon stimulation, 1-kB is phosphorylated and degraded, causing NF-kB to shuttle to the nucleus, thereby activating transcription of target genes. Target genes activated by NF-kB include IL-2, IL-6, GM-CSF, ICAM-1 and class 1 MHC.

Due to its central role and ability to respond to a range of stimuli, reporter constructs utilizing the NF-kB promoter element are used to screen the supernatants produced in Example 7. Activators or inhibitors of NF-kB would be useful in treating diseases. For example, inhibitors of NF-kB could be used to treat those diseases related to the acute or chronic activation of NF-kB, such as rheumatoid arthritis.

To construct a vector containing the NF-kB promoter element, a PCR based strategy is employed. The upstream primer contains four tandem copies of the NF-kB binding site (that is, 5′-GGGGACTTTCCC-3′) (SEQ ID NO:24), 18 bp of sequence complementary to the 5′ end of the SV40 early promoter sequence, and is flanked with an Xho I site: 5′-GCG GCC TCG AGG GGA CTT TCC CGG GGA CTT TCC GGG GAC TTT CCG GGA CTT TCC ATC CTG CCA TCT CAA TTA G-3′ (SEQ ID NO:25).

The downstream primer is complementary to the 3′ end of the SV40 promoter and is flanked with a Hin dIII site: 5′-GCG GCA AGC TTT TTG CAA AGC CTA GGC-3′ (SEQ ID NO:26).

PCR amplification is performed using the SV40 promoter template present in the pb-gal:promoter plasmid obtained from Clontech. The resulting PCR fragment is digested with Xho I and Hin dIII and subcloned into BLSK2-(Stratagene). Sequencing with the T7 and T3 primers confirms the insert contains the following sequence (SEQ ID NO:27):

5′-CTCGAGGGGACTTTCCCGGGGACTTTCCGGGGACTTTCCGGGACTTT
CCATCTGCCATCTCAATTAGTCAGCAACCATAGTCCCGCCCCTAACTCCG
CCCATCCCGCCCCTAACTCCGCCCAGTTCCGCCCATTCTCCGCCCCATGG
CTGACTAATTTTTTTTATTTATGCAGAGGCCGAGGCCGCCTCGGCCTCTG
AGCTATTCCAGAAGTAGTGAGGAGGCTTTTTTGGAGGCCTAGGCTTTTGC
AAAAAGCTT-3′

Next, replace the SV40 minimal promoter element present in the pSEAP2-promoter plasmid (Clontech) with this NF-κB/SV40 fragment using Xho I and Hin dEi. However, this vector does not contain a neomycin resistance gene, and therefore, is not preferred for mammalian expression systems.

In order to generate stable mammalian cell lines, the NF-κB/SV40/SEAP cassette is removed from the above NF-κB/SEAP vector using restriction enzymes Sal I and Not I, and inserted into a vector containing neomycin resistance. Particularly, the NF-kB/SV40/SEAP cassette was inserted into pGFP-1 (Clontech), replacing the GFP gene, after restricting pGFP-1 with Sal I and Not I.

Once NF-kB/SV40/SEAP/Neo vector is created, stable Jurkat T-cells are created and maintained according to the protocol described in Example 9. Similarly, the method for assaying supernatants with these stable Jurkat T-cells is also described in Example 9. As a positive control, exogenous TNF-a (0.1,1, 10 ng) is added to wells H9, H10, and H11, with a 5-10 fold activation typically observed.

### Example 13

### Assay for SEAP Activity

As a reporter molecule for the assays described in Examples 9-12, SEAP activity is assayed using the Tropix Phospho-light Kit (Cat. BP-400) according to the following general procedure. The Tropix Phospho-light Kit supplies the Dilution, Assay, and Reaction Buffers used below.

Prime a dispenser with the 2.5× Dilution Buffer and dispense 15 μl of 2.5× dilution buffer into Optiplates containing 35 μl of a supernatant. Seal the plates with a plastic sealer and incubate at 65° C. for 30 min. Separate the Optiplates to avoid uneven heating.

Cool the samples to room temperature for 15 minutes. Empty the dispenser and prime with the Assay Buffer. Add 50 ml Assay Buffer and incubate at room temperature 5 min. Empty the dispenser and prime with the Reaction Buffer (see the table below). Add 50 ml Reaction Buffer and incubate at room temperature for 20 minutes. Since the intensity of the chemiluminescent signal is time dependent, and it takes about 10 minutes to read 5 plates on luminometer, one should treat 5 plates at each time and start the second set 10 minutes later.

Read the relative light unit in the luminometer. Set H12 as blank, and print the results. An increase in chemiluminescence indicates reporter activity.

Reaction Buffer Formulation:

| # of plates | Rxn buffer diluent (ml) | CSPD (ml) |
|---|---|---|
| 10 | 60 | 3 |
| 11 | 65 | 3.25 |
| 12 | 70 | 3.5 |
| 13 | 75 | 3.75 |
| 14 | 80 | 4 |
| 15 | 85 | 4.25 |
| 16 | 90 | 4.5 |
| 17 | 95 | 4.75 |
| 18 | 100 | 5 |
| 19 | 105 | 5.25 |
| 20 | 110 | 5.5 |
| 21 | 115 | 5.75 |
| 22 | 120 | 6 |
| 23 | 125 | 6.25 |
| 24 | 130 | 6.5 |
| 25 | 135 | 6.75 |
| 26 | 140 | 7 |
| 27 | 145 | 7.25 |
| 28 | 150 | 7.5 |
| 29 | 155 | 7.75 |
| 30 | 160 | 8 |
| 31 | 165 | 8.25 |
| 32 | 170 | 8.5 |
| 33 | 175 | 8.75 |
| 34 | 180 | 9 |
| 35 | 185 | 9.25 |
| 36 | 190 | 9.5 |
| 37 | 195 | 9.75 |
| 38 | 200 | 10 |
| 39 | 205 | 10.25 |
| 40 | 210 | 10.5 |
| 41 | 215 | 10.75 |
| 42 | 220 | 11 |
| 43 | 225 | 11.25 |
| 44 | 230 | 11.5 |
| 45 | 235 | 11.75 |
| 46 | 240 | 12 |
| 47 | 245 | 12.25 |
| 48 | 250 | 12.5 |
| 49 | 255 | 12.75 |
| 50 | 260 | 13 |

### Example 14

### High-Throughput Screening Assay Identifying Changes in Small Molecule Concentration and Membrane Permeability

Binding of a ligand to a receptor is known to alter intracellular levels of small molecules, such as calcium, potassium, sodium, and pH, as well as alter membrane potential. These alterations can be measured in an assay to identify supernatants which bind to receptors of a particular cell. Although the following protocol describes an assay for calcium, this protocol can easily be modified to detect changes in potassium, sodium, pH, membrane potential, or any other small molecule which is detectable by a fluorescent probe.

The following assay uses Fluorometric Imaging Plate Reader (“FLIPR”) to measure changes in fluorescent molecules (Molecular Probes) that bind small molecules. Clearly, any fluorescent molecule detecting a small molecule can be used instead of the calcium fluorescent molecule, fluo-3, used here.

For adherent cells, seed the cells at 10,000-20,000 cells/well in a Co-star black 96-well plate with clear bottom. The plate is incubated in a CO₂ incubator for 20 hours. The adherent cells are washed two times in Biotek washer with 200 μl of HBSS (Hank's Balanced Salt Solution) leaving 100 μl of buffer after the final wash.

A stock solution of 1 mg/ml fluo-3 is made in 10% pluronic acid DMSO. To load the cells with fluo-3, 50 μl of 12 μg/ml fluo-3 is added to each well. The plate is incubated at 37° C. in a CO₂ incubator for 60 min. The plate is washed four times in the Biotek washer with HBSS leaving 100 μl of buffer.

For non-adherent cells, the cells are spun down from culture media. Cells are resuspended to 2-5×10⁶ cells/ml with HBSS in a 50-ml conical tube. 4 μl of 1 mg/ml fluo-3 solution in 10% pluronic acid DMSO is added to each 1 ml of cell suspension. The tube is then placed in a 37° C. water bath for 30-60 min. The cells are washed twice with HBSS, resuspended to 1×10⁶ cells/ml, and dispensed into a microplate, 100 μl/well. The plate is centrifuged at 1000 rpm for 5 min. The plate is then washed once in Denley CellWash with 200 μl, followed by an aspiration step to 100 μl final volume.

For a non-cell based assay, each well contains a fluorescent molecule, such as fluo-3. The supernatant is added to the well, and a change in fluorescence is detected.

To measure the fluorescence of intracellular calcium, the FLIPR is set for the following parameters: (1) System gain is 300-800 mW; (2) Exposure time is 0.4 second; (3) Camera F/stop is F/2; (4) Excitation is 488 nm; (5) Emission is 530 nm; and (6) Sample addition is 50 μl. Increased emission at 530 nm indicates an extracellular signaling event caused by the a molecule, either HFLP or a molecule induced by HFLP, which has resulted in an increase in the intracellular Ca²⁺ concentration.

### Example 15

### High-Throughput Screening Assay Identifying Tyrosine Kinase Activity

The Protein Tyrosine Kinases (PTK) represent a diverse group of transmembrane and cytoplasmic kinases. Within the Receptor Protein Tyrosine Kinase RPTK) group are receptors for a range of mitogenic and metabolic growth factors including the PDGF, FGF, EGF, NGF, HGF and Insulin receptor subfamilies. In addition there are a large family of RPTKs for which the corresponding ligand is unknown. Ligands for RPTKs include mainly secreted small proteins, but also membrane-bound and extracellular matrix proteins.

Activation of RPTK by ligands involves ligand-mediated receptor dimerization, resulting in transphosphorylation of the receptor subunits and activation of the cytoplasmic tyrosine kinases. The cytoplasmic tyrosine kinases include receptor associated tyrosine kinases of the src-family (e.g., src, yes, lck, lyn, fyn) and non-receptor linked and cytosolic protein tyrosine kinases, such as the Jak family, members of which mediate signal transduction triggered by the cytokine superfamily of receptors (e.g., the Interleukins, Interferons, GM-CSF, and Leptin).

Because of the wide range of known factors capable of stimulating tyrosine kinase activity, identifying whether HFLP or a molecule induced by HFLP is capable of activating tyrosine kinase signal transduction pathways is of interest. Therefore, the following protocol is designed to identify such molecules capable of activating the tyrosine kinase signal transduction pathways.

Seed target cells (e.g., primary keratinocytes) at a density of approximately 25,000 cells per well in a 96 well Loprodyne Silent Screen Plates purchased from Nalge Nunc (Naperville, Ill.). The plates are sterilized with two 30 minute rinses with 100% ethanol, rinsed with water and dried overnight. Some plates are coated for 2 hr with 100 ml of cell culture grade type I collagen (50 mg/ml), gelatin (2%) or polylysine (50 mg/ml), all of which can be purchased from Sigma Chemicals (St. Louis, Mo.) or 10% Matrigel purchased from Becton Dickinson (Bedford, Mass.), or calf serum, rinsed with PBS and stored at 4° C. Cell growth on these plates is assayed by seeding 5,000 cells/well in growth medium and indirect quantitation of cell number through use of alamarBlue as described by the manufacturer Alamar Biosciences, Inc. (Sacramento, Calif.) after 48 hr. Falcon plate covers #3071 from Becton Dickinson (Bedford, Mass.) are used to cover the Loprodyne Silent Screen Plates. Falcon Microtest III cell culture plates can also be used in some proliferation experiments.

To prepare extracts, A431 cells are seeded onto the nylon membranes of Loprodyne plates (20,000/200 ml/well) and cultured overnight in complete medium. Cells are quiesced by incubation in serum-free basal medium for 24 hr. After 5-20 minutes treatment with EGF (60 ng/ml) or 50 μl of the supernatant produced in Example 7, the medium was removed and 100 ml of extraction buffer ((20 mM HEPES pH 7.5, 0.15 M NaCl, 1% Triton X-100, 0.1% SDS, 2 mM Na₃VO₄, 2 mM Na₄P₂O₇ and a cocktail of protease inhibitors (# 1836170) obtained from Boeheringer Mannheim (Indianapolis, Ind.) is added to each well and the plate is shaken on a rotating shaker for 5 minutes at 4° C. The plate is then placed in a vacuum transfer manifold and the extract filtered through the 0.45 mm membrane bottoms of each well using house vacuum. Extracts are collected in a 96-well catch/assay plate in the bottom of the vacuum manifold and immediately placed on ice. To obtain extracts clarified by centrifugation, the content of each well, after detergent solubilization for 5 minutes, is removed and centrifuged for 15 minutes at 4° C. at 16,000×g.

Test the filtered extracts for levels of tyrosine kinase activity. Although many methods of detecting tyrosine kinase activity are known, one method is described here.

Generally, the tyrosine kinase activity of a supernatant is evaluated by determining its ability to phosphorylate a tyrosine residue on a specific substrate (a biotinylated peptide). Biotinylated peptides that can be used for this purpose include PSK1 (corresponding to amino acids 6-20 of the cell division kinase cdc2-p34) and PSK2 (corresponding to amino acids 1-17 of gastrin). Both peptides are substrates for a range of tyrosine kinases and are available from Boehringer Mannheim.

The tyrosine kinase reaction is set up by adding the following components in order. First, add 10 ul of 5 uM Biotinylated Peptide, then 10 μl ATP/Mg²⁺ (5 mM ATP/50 mM MgCl₂), then 10 μl of 5× Assay Buffer (40 mM imidazole hydrochloride, pH 7.3, 40 mM b-glycerophosphate, 1 mM EGTA, 100 mM MgCl₂, 5 mM MnCl₂, 0.5 mg/ml BSA), then 5 μl of Sodium Vanadate(1 mM), and then 5 μl of water. Mix the components gently and preincubate the reaction mix at 30° C. for 2 min. Initial the reaction by adding 10 μl of the control enzyme or the filtered supernatant.

The tyrosine kinase assay reaction is then terminated by adding 10 μl of 120 mm EDTA and place the reactions on ice.

Tyrosine kinase activity is determined by transferring 50 μl aliquot of reaction mixture to a microtiter plate (MTP) module and incubating at 37° C. for 20 min. This allows the streptavadin coated 96 well plate to associate with the biotinylated peptide. Wash the MTP module with 300 μl/well of PBS four times. Next add 75 μl of anti-phospotyrosine antibody conjugated to horse radish peroxidase (anti-P-Tyr-POD(0.5 u/ml)) to each well and incubate at 37° C. for one hour. Wash the well as above.

Next add 100 μl of peroxidase substrate solution (Boehringer Mannheim) and incubate at room temperature for at least 5 min (up to 30 min). Measure the absorbance of the sample at 405 nm by using ELISA reader. The level of bound peroxidase activity is quantitated using an ELISA reader and reflects the level of tyrosine kinase activity.

### Example 16

### High-Throughput Screening Assay Identifying Phosphorylation Activity

As a potential alternative and/or compliment to the assay of protein tyrosine kinase activity described in Example 15, an assay which detects activation (phosphorylation) of major intracellular signal transduction intermediates can also be used. For example, as described below one particular assay can detect tyrosine phosphorylation of the Erk-1 and Erk-2 kinases. However, phosphorylation of other molecules, such as Raf, JNK, p38 MAP, Map kinase kinase (MEK), MEK kinase, Src, Muscle specific kinase (MuSK), IRAK, Tec, and Janus, as well as any other phosphoserine, phosphotyrosine, or phosphothreonine molecule, can be detected by substituting these molecules for Erk-1 or Erk-2 in the following assay.

Specifically, assay plates are made by coating the wells of a 96-well ELISA plate with 0.1 ml of protein G (1 μg/ml) for 2 hr at RT. The plates are then rinsed with PBS and blocked with 3% BSA/PBS for 1 hr at RT. The protein G plates are then treated with 2 commercial monoclonal antibodies (100 ng/well) against Erk-1 and Erk-2 (1 hr at RT; obtained from Santa Cruz Biotechnology). To detect other molecules, this step can easily be modified by substituting a monoclonal antibody detecting any of the above described molecules. After 3-5 rinses with PBS, the plates are stored at 4° C. until use.

A431 cells are seeded at 20,000/well in a 96-well Loprodyne filterplate and cultured overnight in growth medium. The cells are then starved for 48 hr in basal medium (DMEM) and then treated with EGF (6 ng/well) or 50 μL of the supernatants obtained in Example 7 for 5-20 minutes. The cells are then solubilized and extracts filtered directly into the assay plate.

After incubation with the extract for 1 hr at RT, the wells are again rinsed. As a positive control, a commercial preparation of MAP kinase (10 ng/well) is used in place of A431 extract. Plates are then treated with a commercial polyclonal (rabbit) antibody (1 μg/ml) which specifically recognizes the phosphorylated epitope of the Erk-1 and Erk-2 kinases (1 hr at RT). This antibody is biotinylated by standard procedures. The bound polyclonal antibody is then quantitated by successive incubations with Europium-streptavidin and Europium fluorescence enhancing reagent in the Wallac DELFIA instrument (time-resolved fluorescence). An increased fluorescent signal over background indicates a phosphorylation by HFLP or a molecule induced by HFLP.

### Example 17

### Method of Determining Alterations in the HFLP Gene

RNA isolated from entire families or individual patients presenting with a phenotype of interest (such as a disease) is be isolated. cDNA is then generated from these RNA samples using protocols known in the art (see, Sambrook, et al., supra) The cDNA is then used as a template for PCR, employing primers surrounding regions of interest in SEQ ID NO:1 or SEQ ID NO:3. Suggested PCR conditions consist of 35 cycles at 95° C. for 30 seconds; 60-120 seconds at 52-58° C.; and 60-120 seconds at 70° C., using buffer solutions described provided by Sidransky and colleagues (Science 252:706 (1991)).

PCR products are then sequenced using primers labeled at their 5′ end with T4 polynucleotide kinase, employing SequiTherm Polymerase (Epicentre Technologies). The intron-exon borders of selected exons of HFLP are also determined and genomic PCR products analyzed to confirm the results. PCR products harboring suspected mutations in HFLP are then cloned and sequenced to validate the results of the direct sequencing.

PCR products of HFLP are cloned into T-tailed vectors as described by Holton and Graham (Nucl. Acids Res. 19:1156 (1991)) and sequenced with T7 polymerase (United States Biochemical). Affected individuals are identified by mutations in HFLP not present in unaffected individuals.

Genomic rearrangements are also observed as a method of determining alterations in the HFLP gene. Genomic clones are nick-translated with digoxigenindeoxy-uridine 5′-triphosphate (Boehringer Manheim), and FISH performed as described by Johnson and coworkers (Meth. Cell Biol. 35:73-99 (1991)). Hybridization with the labeled probe is carried out using a vast excess of human cot-1 DNA for specific hybridization to the HFLP genomic locus.

Chromosomes are counterstained with 4,6-diamino-2-phenylidole and propidium iodide, producing a combination of C- and R-bands. Aligned images for precise mapping are obtained using a triple-band filter set (Chroma Technology, Brattleboro, Vt.) in combination with a cooled charge-coupled device camera (Photometrics, Tucson, Ariz.) and variable excitation wavelength filters (Johnson, C., et al., Genet. Anal. Tech. Appl. 8:75 (1991)). Image collection, analysis and chromosomal fractional length measurements are performed using the ISee Graphical Program System (Inovision Corporation, Durham, N.C.). Chromosome alterations of the genomic region of HFLP (hybridized by the probe) are identified as insertions, deletions, and translocations. These HFLP alterations are used as a diagnostic marker for an associated disease.

It will be clear that the invention may be practiced otherwise than as particularly described in the foregoing description and examples. Numerous modifications and variations of the present invention are possible in light of the above teachings and, therefore, are within the scope of the appended claims.

The entire disclosure of all publications (including patents, patent applications (specifically U.S. Provisional Patent Application No. 60/055,715), journal articles, laboratory manuals, books, or other documents) cited herein are hereby incorporated by reference.

What is claimed is:

1. An isolated nucleic acid molecule nucleic acid molecule comprising a polynucleotide having a nucleotide sequence at least 95% identical to a sequence selected from the group consisting of:
(a) a nucleotide sequence encoding the HFLP polypeptide having the complete amino acid sequence in SEQ ID NO:4 (i.e., positions −43 to 325 of SEQ ID NO:4);
(b) a nucleotide sequence encoding the HFLP polypeptide having the complete amino acid sequence in SEQ ID NO:4 (i.e., positions −42 to 325 of SEQ ID NO:4), excluding the N-terminal methionine;
(c) a nucleotide sequence encoding the predicted mature HFLP polypeptide having the amino acid sequence at positions 1 to 325 in SEQ ID NO:4;
(d) a nucleotide sequence encoding the HFLP polypeptide having the complete amino acid sequence in SEQ ID NO:2 (i.e., positions −19 to 328 of SEQ ID NO:2) plus the N-terminal 12 amino acid signal peptide from the bovine Frzb molecule;
(e) a nucleotide sequence encoding the HFLP polypeptide having the complete amino acid sequence in SEQ ID NO:2 (i.e., positions −19 to 328 of SEQ ID NO:2) plus the N-terminal 12 amino acid signal peptide from the bovine Frzb molecule, excluding the N-terminal methionine;
(f) a nucleotide sequence encoding the HFLP polypeptide having the complete amino acid sequence in SEQ ID NO:2 (i.e., positions −19 to 328 of SEQ ID NO:2);
(g) a nucleotide sequence encoding the predicted mature HFLP polypeptide having the amino acid sequence at positions 1 to 328 in SEQ ID NO:2;
(h) a nucleotide sequence encoding the conserved frizzled domain of the HFLP polypeptide having the amino acid sequence in SEQ ID NO:2 (i.e., positions 6 to 126 of SEQ ID NO:2);
(i) a nucleotide sequence encoding the HFLP polypeptide having the complete amino acid sequence encoded by the cDNA clone contained in ATCC Deposit No. 209140;
(j) a nucleotide sequence encoding the mature HFLP polypeptide having the amino acid sequence encoded by the cDNA clone contained in ATCC Deposit No. 209140;
(k) a nucleotide sequence encoding the frizzled domain of the HFLP polypeptide having the amino acid sequence encoded by the cDNA clone contained in ATCC Deposit No. 209140; and
(l) a nucleotide sequence complementary to any of the nucleotide sequences in (a), (b), (c), (d), (e), (f), (g), (h), (i), (j), or (k), above.

2. The nucleic acid molecule of claim 1 wherein said polynucleotide has the complete nucleotide sequence in FIGS. 1A and 1B (SEQ ID NO:1).

3. The nucleic acid molecule of claim 1 wherein said polynucleotide has the nucleotide sequence in FIGS. 1A and 1B (SEQ ID NO:1) encoding the HFLP polypeptide having the amino acid sequence in positions −19 to 328 of SEQ ID NO:2.

4. The nucleic acid molecule of claim 1 wherein said polynucleotide has the nucleotide sequence in FIGS. 1A and 1B (SEQ ID NO:1) encoding the mature HFLP polypeptide having the amino acid sequence from about 1 to about 328 in SEQ ID NO:2.

5. The nucleic acid molecule of claim 1 wherein said polynucleotide has the nucleotide sequence in FIGS. 1A and 1B (SEQ ID NO:1) encoding the conserved frizzled domain of the HFLP polypeptide having the amino acid sequence from about 6 to about 126 in SEQ ID NO:2.

6. An isolated nucleic acid molecule comprising a polynucleotide having a nucleotide sequence at least 95% identical to a sequence selected from the group consisting of:
(a) a nucleotide sequence encoding a polypeptide comprising the amino acid sequence of residues n¹-328 of SEQ ID NO:2, where n¹ is an integer in the range of-19 to 6;
(b) a nucleotide sequence encoding a polypeptide comprising the amino acid sequence of residues −19-m¹ of SEQ ID NO:2, where m¹ is an integer in the range of 118-328;
(c) a nucleotide sequence encoding a polypeptide having the amino acid sequence consisting of residues n¹-m¹ of SEQ ID NO:2, where n¹ and m¹ are integers as defined respectively in (a) and (b) above; and
(d) a nucleotide sequence encoding a polypeptide consisting of a portion of the complete HFLP amino acid sequence encoded by the cDNA clone contained in ATCC Deposit No. 209140, wherein said portion excludes from 1 to about 25 amino acids from the amino terminus of said complete amino acid sequence encoded by the cDNA clone contained in ATCC Deposit No. 209140;
(e) a nucleotide sequence encoding a polypeptide consisting of a portion of the complete HFLP amino acid sequence encoded by the cDNA clone contained in ATCC Deposit No. 209140 wherein said portion excludes from 1 to about 210 amino acids from the carboxy terminus of said complete amino acid sequence encoded by the cDNA clone contained in ATCC Deposit No. 209140; and
(f) a nucleotide sequence encoding a polypeptide consisting of a portion of the complete HFLP amino acid sequence encoded by the cDNA clone contained in ATCC Deposit No. 209140 wherein said portion include a combination of any of the amino terminal and carboxy terminal deletions in (d) and (e), above.

7. The nucleic acid molecule of claim 1 wherein said polynucleotide has the complete nucleotide sequence of the cDNA clone contained in ATCC Deposit No. 209140.

8. The nucleic acid molecule of claim 1 wherein said polynucleotide has the nucleotide sequence encoding the mature HFLP polypeptide having the amino acid sequence encoded by the cDNA clone contained in ATCC Deposit No. 209140.

9. The nucleic acid molecule of claim 1 wherein said polynucleotide has the nucleotide sequence encoding the conserved frizzled domain of the HFLP polypeptide having the amino acid sequence encoded by the cDNA clone contained in ATCC Deposit No. 209140.

10. An isolated nucleic acid molecule comprising a polynucleotide which hybridizes under stringent hybridization conditions to a polynucleotide having a nucleotide sequence identical to a nucleotide sequence in (a), (b), (c), (d), (e), (f), (g), (h), (i), (j), (k), or (l) of claim 1 wherein said polynucleotide which hybridizes does not hybridize under stringent hybridization conditions to a polynucleotide having a nucleotide sequence consisting of only A residues or of only T residues.

11. An isolated nucleic acid molecule comprising a polynucleotide which encodes the amino acid sequence of an epitope-bearing portion of a HFLP polypeptide having an amino acid sequence in (a), (b), (c), (d), (e), (f), (g), (h), (i), (j), or (k) of claim 1.

12. The isolated nucleic acid molecule of claim 11, which encodes an epitope-bearing portion of a HFLP polypeptide wherein the amino acid sequence of said portion is selected from the group of sequences in SEQ ID NO:2 consisting of: about Pro-1 to about Ala-9; about Met-32 to about Ile-40; about Asn-45 to about Glu-53; about Gln-61 to about Asn-69; about Pro-94 to about Cys-102; about Arg-104 to about Leu-112; about Tyr-132 to about Pro-140; about Asp-146 to about Ile-154; about Pro-158 to about Pro-166; about Asp-170 to about Arg-178; about Lys-180 to about Leu-188; about Gln-208 to about Thr-216; about Ser-227 to about Gln-235; about Trp-277 to about Gln-287; about Thr-305 to about Pro-314; about Ser-312 to about Lys-320; about Thr-321 to about Pro-329; about Thr-305 to about Lys-320; about Pro-314 to about Pro-329; about Lys-303 to about Asn-332; and about Gln-339 to about Val-347.

13. A method for making a recombinant vector comprising inserting an isolated nucleic acid molecule of claim 1 into a vector.

14. A recombinant vector produced by the method of claim 13.

15. A method of making a recombinant host cell comprising introducing the recombinant vector of claim 14 into a host cell.

16. A recombinant host cell produced by the method of claim 15.

17. A recombinant method for producing a HFLP polypeptide, comprising culturing the recombinant host cell of claim 16 under conditions such that said polypeptide is expressed and recovering said polypeptide.

18. An isolated HFLP polypeptide comprising an amino acid sequence at least 95% identical to a sequence selected from the group consisting of:
(a) the amino acid sequence of the full-length HFLP polypeptide having the complete amino acid sequence shown in SEQ ID NO:4 (i.e., positions −43 to 325 of SEQ ID NO:4);
(b) the amino acid sequence of the full-length HFLP polypeptide having the complete amino acid sequence shown in SEQ ID NO:4 (i.e., positions −42 to 325 of SEQ ID NO:4), excluding the N-terminal methionine;
(c) the amino acid sequence of the mature HFLP polypeptide having the complete amino acid sequence in SEQ ID NO:4 (i.e., positions 1 to 325 of SEQ ID NO:4);
(d) the amino acid sequence of the full-length HFLP polypeptide having the complete amino acid sequence shown in SEQ ID NO:2 (i.e., positions −19 to 328 of SEQ ID NO:2) plus the N-terminal 12 amino acid signal peptide from the bovine Frzb molecule;
(e) the amino acid sequence of the full-length HFLP polypeptide having the complete amino acid sequence shown in SEQ ID NO:2 (i.e., positions −19 to 328 of SEQ ID NO:2) plus the N-terminal 12 amino acid signal peptide from the bovine Frzb molecule, excluding the N-terminal methionine;
(f) the amino acid sequence of the HFLP polypeptide having the complete amino acid sequence in SEQ ID NO:2 (i.e., positions −19 to 328 of SEQ ID NO:2);
(g) the amino acid sequence of the predicted mature HFLP polypeptide having the amino acid sequence at positions 1 to 328 in SEQ ID NO:2;
(h) the amino acid sequence of the predicted frizzled domain of the HFLP polypeptide having the amino acid sequence in SEQ ID NO:2 (i.e., positions 6 to 126 of SEQ ID NO:2);
(i) the amino acid sequence of the HFLP polypeptide having the complete amino acid sequence encoded by the cDNA clone contained in ATCC Deposit No. 209140;
(j) the amino acid sequence of the mature HFLP polypeptide having the amino acid sequence encoded by the cDNA clone contained in ATCC Deposit No. 209140; and
(k) the amino acid sequence of the frizzled domain of the HFLP polypeptide having the amino acid sequence encoded by the cDNA clone contained in ATCC Deposit No. 209140.

19. An isolated polypeptide comprising an epitope-bearing portion of the HFLP protein, wherein said portion is selected from the group consisting of: a polypeptide comprising amino acid residues from about Pro-1 to about Ala-9 of SEQ ID NO:2; a polypeptide comprising amino acid residues from about Met-32 to about Ile-40 of SEQ ID NO:2, a polypeptide comprising amino acid residues from about Asn-45 to about Glu-53 of SEQ ID NO:2; a polypeptide comprising amino acid residues from about Gln-61 to about Asn-69 of SEQ ID NO:2; a polypeptide comprising amino acid residues from about Pro-94 to about Cys-102 of SEQ ID NO:2; a polypeptide comprising amino acid residues from about Arg-104 to about Leu-112 of SEQ ID NO:2; a polypeptide comprising amino acid residues from about Tyr-132 to about Pro-140 of SEQ ID NO:2; a polypeptide comprising amino acid residues from about Asp-146 to about Ile-154 of SEQ ID NO:2; a polypeptide comprising amino acid residues from about Pro-158 to about Pro-166 of SEQ ID NO:2; a polypeptide comprising amino acid residues from about Asp-170 to about Arg-178 of SEQ ID NO:2; a polypeptide comprising amino acid residues from about Lys-180 to about Leu-188 of SEQ ID NO:2; a polypeptide comprising amino acid residues from about Gln-208 to about Thr-216 of SEQ ID NO:2; a polypeptide comprising amino acid residues from about Ser-227 to about Gln-235 of SEQ ID NO:2; a polypeptide comprising amino acid residues from about Trp-277 to about Gln-287 of SEQ ID NO:2; a polypeptide comprising amino acid residues from about Thr-305 to about Pro-314 of SEQ ID NO:2; a polypeptide comprising amino acid residues from about Ser-312 to about Lys-320 of SEQ ID NO:2; a polypeptide comprising amino acid residues from about Thr-321 to about Pro-329 of SEQ ID NO:2; a polypeptide comprising amino acid residues from about Thr-305 to about Lys-320 of SEQ ID NO:2; a polypeptide comprising amino acid residues from about Pro-314 to about Pro-329 of SEQ ID NO:2; a polypeptide comprising amino acid residues from about Lys-303 to about Asn-332 of SEQ ID NO:2; and a polypeptide comprising amino acid residues from about Gln-339 to about Val-347 of SEQ ID NO:2.

20. An isolated antibody that binds specifically to a HFLP polypeptide of claim 18.

21. An isolated nucleic acid molecule comprising a polynucleotide having a sequence at least 95% identical to a sequence selected from the group consisting of:
(a) the nucleotide sequence of SEQ ID NO:6;
(b) the nucleotide sequence of SEQ ID NO:7;
(c) the nucleotide sequence of SEQ ID NO:8;
(d) the nucleotide sequence of SEQ ID NO:9;
(e) the nucleotide sequence of SEQ ID NO:10;
(f) the nucleotide sequence of SEQ ID NO:11;
(g) the nucleotide sequence of a portion of the sequence shown in FIGS. 1A and 1B (SEQ ID NO:1) wherein said portion comprises at least 50 contiguous nucleotides from nucleotide 600 to nucleotide 1144;
(h) the nucleotide sequence of a portion of the sequence shown in FIGS. 1A and 1B (SEQ ID NO:1) wherein said portion consists of nucleotides 250-1144, 500-1144, 600-1144, 750-1144, 1000-1144, 250-1000, 500-1000, 600-1000, 750-1000, 250-750, 500-750, 600-750, 250-600, and 500-600; and
(i) a nucleotide sequence complementary to any of the nucleotide sequences in (a), (b), (c), (d), (e), (f), (g), or (h), above.
